(19) United States
(12) Patent Application Publication
(10) Pub. No.: US 2015/0071947 A1
(43) Pub. Date: Mar. 12, 2015

(54) METHODS OF IDENTIFYING GENE ISOFORMS FOR ANTI-CANCER TREATMENTS

(71) Applicant: VERASTEM, INC., Cambridge, MA (US)

(72) Inventors: David T. Weaver, Cambridge, MA (US); Irina Shapiro, Cambridge, MA (US); Daniel W. Paterson, Cambridge, MA (US); Alan G. Derr, Westford, MA (US); Jonathan A. Pachter, Cambridge, MA (US)

(21) Appl. No.: 14/384,000

(22) PCT Filed: Mar. 8, 2013
(86) PCT No.: PCT/US13/29909

§ 371 Date: Sep. 9, 2014

Related U.S. Application Data
(60) Provisional application No. 61/609,036, filed on Mar. 9, 2012.

(51) Int. Cl. C12Q 1/68

(52) U.S. Cl. CPC C12Q 1/6886; C12Q 2600/158; C12Q 2600/106
USPC 424/174.1; 506/9; 506/16

(57) ABSTRACT

Novel methods of classifying subjects as candidates for treatment with agent that inhibits or kills cancer associated mesenchymal cells, tumor initiating cancer cells, or cancer stem cells treatment and subsequent administration of the agent that inhibits or kills cancer associated mesenchymal cells, tumor initiating cancer cells, or cancer stem cells are disclosed within.

### CROSS-REFERENCE TO RELATED APPLICATIONS

This application claims the priority of U.S. provisional application Ser. No. 61/609,036, filed Mar. 9, 2012, which is incorporated by reference herein in its entirety.

### BACKGROUND

Currently available therapeutic regimens are ineffective in treating many cancers. Cancer stem cells (CSCs), cancer associated mesenchymal cells, or tumor initiating cancer cells, comprise a unique subpopulation of a tumor and have been identified in a large variety of cancer types. Although this subpopulation of cells constitutes only a small fraction of a tumor, they are thought to be the main cancer cells responsible for tumor initiation, growth, and recurrence. Given that current cancer treatments have, in large part, been designed to target rapidly proliferating cells, this subpopulation of cells, which is often slow growing, may be relatively more resistant to these treatments. Therefore, methods to identify cancer patients likely to respond positively to a treatment comprising an agent that inhibits or kills cancer associated mesenchymal cells, tumor initiating cancer cells, or cancer stem cells are needed; and can provide the basis for subsequent administration of a treatment comprising an agent that inhibits or kills cancer associated mesenchymal cells, tumor initiating cancer cells, or cancer stem cells; to this candidate group of cancer patients.

### SUMMARY OF INVENTION

The present invention provides a method for classifying subjects more likely to respond to a particular therapeutic regimen for treating cancer. The method is based, at least in part, on the characterization of signals (e.g., the level of expression of a gene isoform) possessed by a candidate subject population for treatment with a preselected drug. In general, the method involves identifying differences in candidate and non-candidate subject populations, where for example, a subject population has a gene expression profile associated with a candidate or non-candidate classification. The method can further comprise administration of the therapeutic regimen to the candidate population based on the characterized gene expression profile.

In an aspect, the invention features a method of evaluating or treating a subject, comprising: (a) optionally, acquiring a subject sample, e.g., a tissue sample, such as a biopsy; bodily fluids, such as blood or plasma (b) acquiring a value or values that is a function of the level of expression of a plurality of gene isoforms from a plurality of genes selected from a first and/or second and/or third and/or fourth and/or fifth and/or sixth and/or eighth and/or ninth and/or tenth and/or eleventh and/or twelfth and/or thirteenth set of gene isoforms; (c) responsive to said value or values (i) classifying the subject, e.g., classifying the subject as a candidate or non-candidate for treatment with a preselected drug, and/or treating, or withholding treatment from, the subject with a preselected drug; or (ii) administering a treatment comprising an agent that inhibits or kills cancer associated mesenchymal cells, tumor initiating cancer cells, or cancer stem cells; to said subject; provided that, if (c)(ii) is not performed the acquisition in (a) or (b) comprises directly acquiring; thereby evaluating or treating the subject.

In an embodiment, the invention features, responsive to said value or values, classifying the subject, e.g., classifying the subject as a candidate or non-candidate for treatment with a preselected drug, and/or treating, or withholding treatment from, the subject with a preselected drug, wherein the subject sample is directly acquired, thereby evaluating the subject.

In an embodiment, the invention features, responsive to said value or values, classifying the subject, e.g., classifying the subject as a candidate or non-candidate for treatment with a preselected drug, and/or treating, or withholding treatment from, the subject with a preselected drug, wherein said value or values is directly acquired thereby evaluating the subject.

In an embodiment, the invention features, responsive to said value or values, classifying the subject, e.g., classifying the subject as a candidate or non-candidate for treatment with a preselected drug, and/or treating, or withholding treatment from, the subject with a preselected drug, wherein the subject sample and said value or values are directly acquired thereby evaluating the subject.

In an embodiment, the invention features, responsive to said value or values, administering a treatment comprising an agent that inhibits or kills cancer associated mesenchymal cells, tumor initiating cancer cells, or cancer stem cells to said subject.

In an embodiment, the invention features, responsive to said value or values, classifying the subject, e.g., classifying the subject as a candidate or non-candidate for treatment with a preselected drug, and/or treating, or withholding treatment from, the subject with a preselected drug; and administering a treatment comprising an agent that inhibits or kills cancer associated mesenchymal cells, tumor initiating cancer cells, or cancer stem cells to said subject.

In an embodiment, the first set of gene isoforms (gene isoform set 1) comprises or consists of the gene isoforms in Table 1, Table 2, Table 3, Table 4, Table 5, Table 6, Table 8, Table 9, Table 10, Table 11, Table 12, and Table 13; the second set of gene isoforms (gene isoform set 2) comprises or consist of the gene isoforms in Table 1; the third set of gene isoforms (gene isoform set 3) comprises or consists of the gene isoforms in Table 2; the fourth set of genes (gene isoform set 4) comprises or consists of the gene isoforms in Table 3; the fifth set of gene isoforms (gene isoform set 5) comprises or consists of the gene isoforms in Table 4; and the sixth set of gene isoforms (gene isoform set 6) comprises or consists of the gene isoforms in Table 5; and the seventh set of gene isoforms (gene isoform set 7) comprises or consists of the gene isoforms in Table 6; and the eighth set of gene isoforms (gene isoform set 8) comprises or consists of the gene isoforms in Table 8; and the ninth set of gene isoforms (gene isoform set 9) comprises or consists of the gene isoforms in Table 9; and the tenth set of gene isoforms (gene isoform set 10) comprises or consists of the gene isoforms in Table 10; and the eleventh set of gene isoforms (gene isoform set 11) comprises or consists of the gene isoforms in Table 11; and the twelfth set of gene isoforms (gene isoform set 12) comprises or consists of the gene isoforms in Table 12; and the thirteenth set of gene isoforms (gene isoform set 13) comprises or consists of the gene isoforms in Table 13.

TABLE 1
Gene Isoform Set 1.

| Gene Isoform | | Transcript | | mRNA- |
|---|---|---|---|---|
| (Gene:Probeset) | Description | Cluster Id | Exon ID | Accession |
| AC007276.5:2995046 | | 2995045 | 423639 | NR_027768 |
| AP1S2:4000709 | adaptor-related protein complex 1, | 4000704 | 1040261 | NM_003916 |
| | sigma 2 subunit [Source: HGNC | | | |
| | Symbol; Acc: 560] | | | |
| AP1S2:4000708 | adaptor-related protein complex 1, | 4000704 | 1040261 | NM_003916 |
| | sigma 2 subunit [Source: HGNC | | | |
| | Symbol; Acc: 560] | | | |
| ARRDC1:3195387 | arrestin domain containing 1 | 3195363 | 548677 | ENST00000431925 |
| | [Source: HGNC Symbol; Acc: 28633] | | | |
| ARRDC1:3195397 | arrestin domain containing 1 | 3195363 | 548679 | NM_152285 |
| | [Source: HGNC Symbol; Acc: 28633] | | | |
| ATP2C2:3671770 | ATPase, Ca++ transporting, type 2C, | 3671727 | 842490 | NM_014861 |
| | member 2 [Source: HGNC | | | |
| | Symbol; Acc: 29103] | | | |
| ATP2C2:3671775 | ATPase, Ca++ transporting, type 2C, | 3671727 | 842494 | NM_014861 |
| | member 2 [Source: HGNC | | | |
| | Symbol; Acc: 29103] | | | |
| ATP2C2:3671792 | ATPase, Ca++ transporting, type 2C, | 3671727 | 842499 | NM_014861 |
| | member 2 [Source: HGNC | | | |
| | Symbol; Acc: 29103] | | | |
| CHST2:2646146 | carbohydrate (N-acetylglucosamine-6- | 2646125 | 205977 | NM_004267 |
| | O) sulfotransferase 2 [Source: HGNC | | | |
| | Symbol; Acc: 1970] | | | |
| CLSTN1:2395913 | calsyntenin 1 [Source: HGNC | 2395890 | 49543 | NM_001009566 |
| | Symbol; Acc: 17447] | | | |
| COL5A1:3193523 | collagen, type V, alpha 1 | 3193482 | 547645 | NM_000093 |
| | [Source: HGNC Symbol; Acc: 2209] | | | |
| CYBASC3:3375317 | cytochrome b, ascorbate dependent 3 | 3375307 | 659853 | NM_001161454 |
| | [Source: HGNC Symbol; Acc: 23014] | | | |
| DDAH1:2420905 | dimethylarginine | 2420832 | 64979 | NM_001134445 |
| | dimethylaminohydrolase 1 | | | |
| | [Source: HGNC Symbol; Acc: 2715] | | | |
| DDR1:2901971 | discoidin domain receptor tyrosine | 2901970 | 365880 | ENST00000324771 |
| | kinase 1 [Source: HGNC | | | |
| | Symbol; Acc: 2730] | | | |
| DST:2958471 | dystonin [Source: HGNC | 2958325 | 400789 | NM_015548 |
| | Symbol; Acc: 1090] | | | |
| EPN3:3726550 | epsin 3 [Source: HGNC | 3726537 | 875206 | NM_017957 |
| | Symbol; Acc: 18235] | | | |
| EPPK1:3157889 | epiplakin 1 [Source: HGNC | 3157887 | 525854 | GENSCAN00000018207 |
| | Symbol; Acc: 15577] | | | |
| ESRP2:3696259 | epithelial splicing regulatory protein 2 | 3696226 | 857087 | NM_024939 |
| | [Source: HGNC Symbol; Acc: 26152] | | | |
| GRHL1:2469161 | grainyhead-like 1 (Drosophila) | 2469157 | 94458 | NM_198182 |
| | [Source: HGNC Symbol; Acc: 17923] | | | |
| HRH1:2610723 | histamine receptor H1 [Source: HGNC | 2610707 | 183808 | NM_001098213 |
| | Symbol; Acc: 5182] | | | |
| KIAA1543:3818983 | KIAA1543 [Source: HGNC | 3818973 | 932035 | NM_001080429 |
| | Symbol; Acc: 29307] | | | |
| KRT8P25:2631888 | keratin 8 pseudogene 25 | 2631878 | 196964 | ENST00000473150 |
| | [Source: HGNC Symbol; Acc: 33377] | | | |
| LLGL2:3734949 | lethal giant larvae homolog 2 | 3734903 | 880398 | NM_004524 |
| | (Drosophila) [Source: HGNC | | | |
| | Symbol; Acc: 6629] | | | |
| MARK3:3553750 | MAP/microtubule affinity-regulating | 3553690 | 770187 | NM_001128918 |
| | kinase 3 [Source: HGNC | | | |
| | Symbol; Acc: 6897] | | | |
| MPZL3:3393718 | myelin protein zero-like 3 | 3393704 | 671109 | NM_198275 |
| | [Source: HGNC Symbol; Acc: 27279] | | | |
| MRC2:3730341 | mannose receptor, C type 2 | 3730322 | 877594 | NM_006039 |
| | [Source: HGNC Symbol; Acc: 16875] | | | |
| PNMA2:3128733 | paraneoplastic antigen MA2 | 3128731 | 507391 | NM_007257 |
| | [Source: HGNC Symbol; Acc: 9159] | | | |
| PRKCDBP:3360804 | protein kinase C, delta binding protein | 3360800 | 651142 | NM_145040 |
| | [Source: HGNC Symbol; Acc: 9400] | | | |
| PROM2:2493969 | prominin 2 [Source: HGNC | 2493943 | 110133 | NM_001165978 |
| | Symbol; Acc: 20685] | | | |
| PTGFR:2343426 | prostaglandin F receptor (FP) | 2343418 | 17497 | NM_000959 |
| | [Source: HGNC Symbol; Acc: 9600] | | | |
| RFX2:3847614 | regulatory factor X, 2 (influences HLA | 3847590 | 948347 | AK093575 |
| | class II expression) [Source: HGNC | | | |
| | Symbol; Acc: 9983] | | | |
| SULT1A2:3654687 | sulfotransferase family, cytosolic, 1A, | 3654669 | 832187 | BC052280 |
| | phenol-preferring, member 2 | | | |
| | [Source: HGNC Symbol; Acc: 11454] | | | |
| SULT2B1:3837879 | sulfotransferase family, cytosolic, 2B, | 3837866 | 942962 | NM_004605 |
| | member 1 [Source: HGNC | | | |
| | Symbol; Acc: 11459] | | | |
| SYDE1:3823038 | synapse defective 1, Rho GTPase, | 3823019 | 934308 | NM_033025 |
| | homolog 1 (C. elegans) [Source: HGNC | | | |
| | Symbol; Acc: 25824] | | | |
| SYDE1:3823040 | synapse defective 1, Rho GTPase, | 3823019 | 934308 | NM_033025 |
| | homolog 1 (C. elegans) [Source: HGNC | | | |
| | Symbol; Acc: 25824] | | | |
| SYDE1:3823041 | synapse defective 1, Rho GTPase, | 3823019 | 934308 | NM_033025 |
| | homolog 1 (C. elegans) [Source: HGNC | | | |
| | Symbol; Acc: 25824] | | | |
| TMEM158:2671790 | transmembrane protein 158 | 2671787 | 222082 | NM_015444 |
| | (gene/pseudogene) [Source: HGNC | | | |
| | Symbol; Acc: 30293] | | | |
| TMEM184A:3035399 | transmembrane protein 184A | 3035380 | 448744 | NM_001097620 |
| | [Source: HGNC Symbol; Acc: 28797] | | | |
| TTC9:3542598 | tetratricopeptide repeat domain 9 | 3542596 | 763200 | NM_015351 |
| | [Source: HGNC Symbol; Acc: 20267] | | | |
| VGLL4:2663005 | vestigial like 4 (Drosophila) | 2662956 | 216550 | NM_001128219 |
| | [Source: HGNC Symbol; Acc: 28966] | | | |

TABLE 2
Gene Isoform Set 2.

| Gene Isoform | | Transcript | | |
|---|---|---|---|---|
| (Gene:Probeset) | Description | Cluster Id | Exon ID | mRNA - Accession |
| AC010900.1:2595427 | | 2595388 | 174080 | ENST00000425226 |
| AC097468.6:2599630 | | 2599628 | 176803 | ENST00000432100 |
| ANXA9:2358607 | annexin A9 [Source: HGNC | 2358591 | 26729 | NM_003568 |
| | Symbol; Acc: 547] | | | |
| ANXA9:2358608 | annexin A9 [Source: HGNC | 2358591 | 26730 | NM_003568 |
| | Symbol; Acc: 547] | | | |
| ARHGAP8:3948366 | Rho GTPase activating protein 8 | 3948259 | 1008591 | ENST00000460809 |
| | [Source: HGNC Symbol; Acc: 677] | | | |
| ATP2C2:3671781 | ATPase, Ca++ transporting, type 2C, | 3671727 | 842497 | NM_014861 |
| | member 2 [Source: HGNC | | | |
| | Symbol; Acc: 29103] | | | |
| ATP2C2:3671793 | ATPase, Ca++ transporting, type 2C, | 3671727 | 842499 | NM_014861 |
| | member 2 [Source: HGNC | | | |
| | Symbol; Acc: 29103] | | | |
| ATP2C2:3671798 | ATPase, Ca++ transporting, type 2C, | 3671727 | 842501 | NM_014861 |
| | member 2 [Source: HGNC | | | |
| | Symbol; Acc: 29103] | | | |
| ATP2C2:3671751 | ATPase, Ca++ transporting, type 2C, | 3671727 | 842475 | NM_014861 |
| | member 2 [Source: HGNC | | | |
| | Symbol; Acc: 29103] | | | |
| BRWD1:3932263 | bromodomain and WD repeat domain | 3932261 | 999124 | NR_033800 |
| | containing 1 [Source: HGNC | | | |
| | Symbol; Acc: 12760] | | | |
| C17orf28:3770534 | chromosome 17 open reading frame 28 | 3770512 | 901756 | NM_030630 |
| | [Source: HGNC Symbol; Acc: 15736] | | | |
| C17orf28:3770529 | chromosome 17 open reading frame 28 | 3770512 | 901753 | NM_030630 |
| | [Source: HGNC Symbol; Acc: 15736] | | | |
| C17orf28:3770527 | chromosome 17 open reading frame 28 | 3770512 | 901753 | NM_030630 |
| | [Source: HGNC Symbol; Acc: 15736] | | | |
| C17orf28:3770513 | chromosome 17 open reading frame 28 | 3770512 | 901743 | NM_030630 |
| | [Source: HGNC Symbol; Acc: 15736] | | | |
| C17orf28:3770546 | chromosome 17 open reading frame 28 | 3770512 | 901763 | NM_030630 |
| | [Source: HGNC Symbol; Acc: 15736] | | | |
| C17orf28:3770545 | chromosome 17 open reading frame 28 | 3770512 | 901762 | NM_030630 |
| | [Source: HGNC Symbol; Acc: 15736] | | | |
| C17orf28:3770539 | chromosome 17 open reading frame 28 | 3770512 | 901759 | NM_030630 |
| | [Source: HGNC Symbol; Acc: 15736] | | | |
| C1orf210:2409280 | chromosome 1 open reading frame 210 | 2409275 | 57685 | NM_182517 |
| | [Source: HGNC Symbol; Acc: 28755] | | | |
| C20orf54:3894379 | chromosome 20 open reading frame 54 | 3894365 | 975899 | NM_033409 |
| | [Source: HGNC Symbol; Acc: 16187] | | | |
| CAPN13:2546811 | calpain 13 [Source: HGNC | 2546795 | 143354 | AK026692 |
| | Symbol; Acc: 16663] | | | |
| CCDC64B:3677373 | coiled-coil domain containing 64B | 3677372 | 845774 | NM_001103175 |
| | [Source: HGNC Symbol; Acc: 33584] | | | |
| CTC-362D12.1:2880117 | | 2880051 | 352687 | ENST00000515599 |
| CTD-2048F20.1:2873211 | | 2873168 | 348379 | ENST00000508125 |
| DDR1:2901984 | discoidin domain receptor tyrosine | 2901970 | 365889 | NM_001954 |
| | kinase 1 [Source: HGNC | | | |
| | Symbol; Acc: 2730] | | | |
| DNMT3B:3882062 | DNA (cytosine-5-)-methyltransferase 3 | 3882012 | 968365 | NM_006892 |
| | beta [Source: HGNC Symbol; Acc: 2979] | | | |
| ENAH:2458376 | enabled homolog (Drosophila) | 2458338 | 87633 | NM_001008493 |
| | [Source: HGNC Symbol; Acc: 18271] | | | |
| ENTPD2:3230753 | ectonucleoside triphosphate | 3230733 | 570539 | NM_203468 |
| | diphosphohydrolase 2 [Source: HGNC | | | |
| | Symbol; Acc: 3364] | | | |
| EPHA1:3077346 | EPH receptor A1 [Source: HGNC | 3077321 | 475033 | NM_005232 |
| | Symbol; Acc: 3385] | | | |
| EPN3:3726561 | epsin 3 [Source: HGNC | 3726537 | 875212 | NM_017957 |
| | Symbol; Acc: 18235] | | | |
| EPN3:3726544 | epsin 3 [Source: HGNC | 3726537 | 875203 | NM_017957 |
| | Symbol; Acc: 18235] | | | |
| EPN3:3726547 | epsin 3 [Source: HGNC | 3726537 | 875204 | NM_017957 |
| | Symbol; Acc: 18235] | | | |
| EPN3:3726552 | epsin 3 [Source: HGNC | 3726537 | 875208 | NM_017957 |
| | Symbol; Acc: 18235] | | | |
| EPPK1:3157888 | epiplakin 1 [Source: HGNC | 3157887 | 525853 | AL137725 |
| | Symbol; Acc: 15577] | | | |
| EPS8L1:3841962 | EPS8-like 1 [Source: HGNC | 3841949 | 945192 | NM_133180 |
| | Symbol; Acc: 21295] | | | |
| ESRP2:3696237 | epithelial splicing regulatory protein 2 | 3696226 | 857075 | NM_024939 |
| | [Source: HGNC Symbol; Acc: 26152] | | | |
| ESRP2:3696256 | epithelial splicing regulatory protein 2 | 3696226 | 857084 | NM_024939 |
| | [Source: HGNC Symbol; Acc: 26152] | | | |
| ESRP2:3696254 | epithelial splicing regulatory protein 2 | 3696226 | 857082 | NM_024939 |
| | [Source: HGNC Symbol; Acc: 26152] | | | |
| FNIP1:2874900 | folliculin interacting protein 1 | 2874794 | 349472 | NM_133372 |
| | [Source: HGNC Symbol; Acc: 29418] | | | |
| GRHL1:2469198 | grainyhead-like 1 (Drosophila) | 2469157 | 94485 | NM_198182 |
| | [Source: HGNC Symbol; Acc: 17923] | | | |
| GRHL1:2469199 | grainyhead-like 1 (Drosophila) | 2469157 | 94485 | NM_198182 |
| | [Source: HGNC Symbol; Acc: 17923] | | | |
| GRHL1:2469172 | grainyhead-like 1 (Drosophila) | 2469157 | 94463 | NM_198182 |
| | [Source: HGNC Symbol; Acc: 17923] | | | |
| GRHL1:2469174 | grainyhead-like 1 (Drosophila) | 2469157 | 94464 | NM_198182 |
| | [Source: HGNC Symbol; Acc: 17923] | | | |
| IRF6:2453889 | interferon regulatory factor 6 | 2453881 | 84827 | NM_006147 |
| | [Source: HGNC Symbol; Acc: 6121] | | | |
| KIAA1217:3239076 | KIAA1217 [Source: HGNC | 3238962 | 575758 | NM_019590 |
| | Symbol; Acc: 25428] | | | |
| KIAA1217:3239054 | KIAA1217 [Source: HGNC | 3238962 | 575738 | NM_019590 |
| | Symbol; Acc: 25428] | | | |
| KIAA1217:3239055 | KIAA1217 [Source: HGNC | 3238962 | 575738 | NM_019590 |
| | Symbol; Acc: 25428] | | | |
| KIAA1217:3239075 | KIAA1217 [Source: HGNC | 3238962 | 575757 | NM_019590 |
| | Symbol; Acc: 25428] | | | |
| KIAA1543:3819009 | KIAA1543 [Source: HGNC | 3818973 | 932052 | NM_001080429 |
| | Symbol; Acc: 29307] | | | |
| KIAA1543:3819010 | KIAA1543 [Source: HGNC | 3818973 | 932053 | NM_001080429 |
| | Symbol; Acc: 29307] | | | |
| KRT18P16:2826616 | keratin 18 pseudogene 16 | 2826550 | 319473 | ENST00000510337 |
| | [Source: HGNC Symbol; Acc: 33384] | | | |
| KRT8P12:2650338 | keratin 8 pseudogene 12 [Source: HGNC | 2650322 | 208594 | BC125159 |
| | Symbol; Acc: 28057] | | | |
| KRT8P25:2631889 | keratin 8 pseudogene 25 [Source: HGNC | 2631878 | 196964 | ENST00000473150 |
| | Symbol; Acc: 33377] | | | |
| KRT8P25:2631883 | keratin 8 pseudogene 25 [Source: HGNC | 2631878 | 196962 | ENST00000473150 |
| | Symbol; Acc: 33377] | | | |
| KRT8P25:2631884 | keratin 8 pseudogene 25 [Source: HGNC | 2631878 | 196963 | ENST00000473150 |
| | Symbol; Acc: 33377] | | | |
| KRT8P28:2435385 | keratin 8 pseudogene 28 [Source: HGNC | 2435383 | 73787 | ENST00000433288 |
| | Symbol; Acc: 33380] | | | |
| LEPRE1:2409052 | leucine proline-enriched proteoglycan | 2409004 | 57547 | NM_022356 |
| | (leprecan) 1 [Source: HGNC | | | |
| | Symbol; Acc: 19316] | | | |
| LIMA1:3454369 | LIM domain and actin binding 1 | 3454331 | 708421 | NM_001113546 |
| | [Source: HGNC Symbol; Acc: 24636] | | | |
| LIMA1:3454368 | LIM domain and actin binding 1 | 3454331 | 708421 | NM_001113546 |
| | [Source: HGNC Symbol; Acc: 24636] | | | |
| LIMA1:3454365 | LIM domain and actin binding 1 | 3454331 | 708419 | NM_001113546 |
| | [Source: HGNC Symbol; Acc: 24636] | | | |
| LIMK2:3942847 | LIM domain kinase 2 [Source: HGNC | 3942838 | 1005245 | NM_001031801 |
| | Symbol; Acc: 6614] | | | |
| LLGL2:3734929 | lethal giant larvae homolog 2 | 3734903 | 880385 | NM_004524 |
| | (Drosophila) [Source: HGNC | | | |
| | Symbol; Acc: 6629] | | | |
| LLGL2:3734943 | lethal giant larvae homolog 2 | 3734903 | 880395 | NM_004524 |
| | (Drosophila) [Source: HGNC | | | |
| | Symbol; Acc: 6629] | | | |
| LLGL2:3734961 | lethal giant larvae homolog 2 | 3734903 | 880403 | NM_004524 |
| | (Drosophila) [Source: HGNC | | | |
| | Symbol; Acc: 6629] | | | |
| LLGL2:3734924 | lethal giant larvae homolog 2 | 3734903 | 880385 | NM_004524 |
| | (Drosophila) [Source: HGNC | | | |
| | Symbol; Acc: 6629] | | | |
| MRC2:3730351 | mannose receptor, C type 2 | 3730322 | 877603 | NM_006039 |
| | [Source: HGNC Symbol; Acc: 16875] | | | |
| OVOL1:3335585 | ovo-like 1 (Drosophila) [Source: HGNC | 3335571 | 635841 | NM_004561 |
| | Symbol; Acc: 8525] | | | |
| OVOL1:3335589 | ovo-like 1 (Drosophila) [Source: HGNC | 3335571 | 635844 | NM_004561 |
| | Symbol; Acc: 8525] | | | |
| PROM2:2493972 | prominin 2 [Source: HGNC | 2493943 | 110136 | NM_001165978 |
| | Symbol; Acc: 20685] | | | |
| PROM2:2493975 | prominin 2 [Source: HGNC | 2493943 | 110139 | NM_001165978 |
| | Symbol; Acc: 20685] | | | |
| PROM2:2493976 | prominin 2 [Source: HGNC | 2493943 | 110140 | NM_001165978 |
| | Symbol; Acc: 20685] | | | |
| PROM2:2493946 | prominin 2 [Source: HGNC | 2493943 | 110117 | NM_001165978 |
| | Symbol; Acc: 20685] | | | |
| PSD4:2501284 | pleckstrin and Sec7 domain containing 4 | 2501238 | 114656 | NM_012455 |
| | [Source: HGNC Symbol; Acc: 19096] | | | |
| PSD4:2501285 | pleckstrin and Sec7 domain containing 4 | 2501238 | 114657 | NM_012455 |
| | [Source: HGNC Symbol; Acc: 19096] | | | |
| PTGFR:2343424 | prostaglandin F receptor (FP) | 2343418 | 17496 | NM_000959 |
| | [Source: HGNC Symbol; Acc: 9600] | | | |
| RGL2:2950619 | ral guanine nucleotide dissociation | 2950590 | 395978 | ENST00000494807 |
| | stimulator-like 2 [Source: HGNC | | | |
| | Symbol; Acc: 9769] | | | |
| RP11-24H2.1:3490958 | | 3490947 | 731119 | ENST00000428983 |
| RP11-429J17.6:3119845 | | 3119826 | 501803 | AK125852 |
| RP11-429J17.6:3119847 | | 3119826 | 501803 | AK125852 |
| RP11-429J17.6:3119851 | | 3119826 | 501803 | AK125852 |
| RP11-429J17.6:3119853 | | 3119826 | 501803 | AK125852 |
| RP11-429J17.6:3119855 | | 3119826 | 501803 | NR_033849 |
| RP11-543F8.1:3276725 | | 3276699 | 599323 | ENST00000451609 |
| SLK:3262461 | STE20-like kinase [Source: HGNC | 3262433 | 590321 | NM_014720 |
| | Symbol; Acc: 11088] | | | |
| SULT1A1:3654637 | sulfotransferase family, cytosolic, 1A, | 3654614 | 832163 | NM_001055 |
| | phenol-preferring, member 1 | | | |
| | [Source: HGNC Symbol; Acc: 11453] | | | |
| SULT1A2:3654678 | sulfotransferase family, cytosolic, 1A, | 3654669 | 832184 | NM_001054 |
| | phenol-preferring, member 2 | | | |
| | [Source: HGNC Symbol; Acc: 11454] | | | |
| SYDE1:3823023 | synapse defective 1, Rho GTPase, | 3823019 | 934303 | NM_033025 |
| | homolog 1 (C. elegans) [Source: HGNC | | | |
| | Symbol; Acc: 25824] | | | |
| TJP2:3173885 | tight junction protein 2 (zona occludens | 3173880 | 535835 | NM_001170414 |
| | 2) [Source: HGNC Symbol; Acc: 11828] | | | |
| TJP3:3817150 | tight junction protein 3 (zona occludens | 3817116 | 930910 | NM_014428 |
| | 3) [Source: HGNC Symbol; Acc: 11829] | | | |
| TJP3:3817133 | tight junction protein 3 (zona occludens | 3817116 | 930898 | NM_014428 |
| | 3) [Source: HGNC Symbol; Acc: 11829] | | | |
| TRPV6:3077083 | transient receptor potential cation | 3077072 | 474880 | NM_018646 |
| | channel, subfamily V, member 6 | | | |
| | [Source: HGNC Symbol; Acc: 14006] | | | |
| TTBK2:3620830 | tau tubulin kinase 2 [Source: HGNC | 3620799 | 811328 | AF525400 |
| | Symbol; Acc: 19141] | | | |
| VPS39:3620507 | vacuolar protein sorting 39 homolog (S. | 3620457 | 811128 | ENST00000348544 |
| | cerevisiae) [Source: HGNC | | | |
| | Symbol; Acc: 20593] | | | |

TABLE 3
Gene Isoform Set 3.

| Gene Isoform | | Transcript | | |
|---|---|---|---|---|
| (Gene:Probeset) | Description | Cluster Id | Exon ID | mRNA - Accession |
| PFAS:3709579 | phosphoribosylformylglycinamidine | 3709540 | 865047 | NM_012393 |
| | synthase [Source: HGNC | | | |
| | Symbol; Acc: 8863] | | | |
| PFAS:3709581 | phosphoribosylformylglycinamidine | 3709540 | 865047 | NM_012393 |
| | synthase [Source: HGNC | | | |
| | Symbol; Acc: 8863] | | | |
| NAALADL2:2653208 | N-acetylated alpha-linked acidic | 2653114 | 210440 | ENST00000489299 |
| | dipeptidase-like 2 [Source: HGNC | | | |
| | Symbol; Acc: 23219] | | | |
| PFAS:3709553 | phosphoribosylformylglycinamidine | 3709540 | 865029 | NM_012393 |
| | synthase [Source: HGNC | | | |
| | Symbol; Acc: 8863] | | | |
| EEF1D:3157636 | eukaryotic translation elongation factor | 3157596 | 525707 | NM_001130057 |
| | 1 delta (guanine nucleotide exchange | | | |
| | protein) [Source: HGNC | | | |
| | Symbol; Acc: 3211] | | | |
| PFAS:3709543 | phosphoribosylformylglycinamidine | 3709540 | 865022 | NM_012393 |
| | synthase [Source: HGNC | | | |
| | Symbol; Acc: 8863] | | | |
| PFAS:3709547 | phosphoribosylformylglycinamidine | 3709540 | 865026 | NM_012393 |
| | synthase [Source: HGNC | | | |
| | Symbol; Acc: 8863] | | | |
| ZIC2:3498788 | Zic family member 2 (odd-paired | 3498780 | 736058 | NM_007129 |
| | homolog, Drosophila) [Source: HGNC | | | |
| | Symbol; Acc: 12873] | | | |
| PFAS:3709552 | phosphoribosylformylglycinamidine | 3709540 | 865028 | NM_012393 |
| | synthase [Source: HGNC | | | |
| | Symbol; Acc: 8863] | | | |
| FHOD3:3784894 | formin homology 2 domain containing 3 | 3784840 | 910488 | NM_025135 |
| | [Source: HGNC Symbol; Acc: 26178] | | | |
| NAALADL2:2653150 | N-acetylated alpha-linked acidic | 2653114 | 210389 | ENST00000489299 |
| | dipeptidase-like 2 [Source: HGNC | | | |
| | Symbol; Acc: 23219] | | | |
| RRP9:2675774 | ribosomal RNA processing 9, small | 2675763 | 224388 | NM_004704 |
| | subunit (SSU) processome component, | | | |
| | homolog (yeast) [Source: HGNC | | | |
| | Symbol; Acc: 16829] | | | |
| NNT:2808443 | nicotinamide nucleotide | 2808438 | 307897 | NM_012343 |
| | transhydrogenase [Source: HGNC | | | |
| | Symbol; Acc: 7863] | | | |
| PFAS:3709580 | phosphoribosylformylglycinamidine | 3709540 | 865047 | NM_012393 |
| | synthase [Source: HGNC | | | |
| | Symbol; Acc: 8863] | | | |
| PIK3IP1:3957808 | phosphoinositide-3-kinase interacting | 3957790 | 1014242 | NM_052880 |
| | protein 1 [Source: HGNC | | | |
| | Symbol; Acc: 24942] | | | |
| PFAS:3709542 | phosphoribosylformylglycinamidine | 3709540 | 865021 | NM_012393 |
| | synthase [Source: HGNC | | | |
| | Symbol; Acc: 8863] | | | |
| RUNX1:3930506 | runt-related transcription factor 1 | 3930360 | 998038 | NM_001754 |
| | [Source: HGNC Symbol; Acc: 10471] | | | |
| PFAS:3709584 | phosphoribosylformylglycinamidine | 3709540 | 865047 | NM_012393 |
| | synthase [Source: HGNC | | | |
| | Symbol; Acc: 8863] | | | |
| PFAS:3709586 | phosphoribosylformylglycinamidine | 3709540 | 865047 | NM_012393 |
| | synthase [Source: HGNC | | | |
| | Symbol; Acc: 8863] | | | |
| FHOD3:3784879 | formin homology 2 domain containing 3 | 3784840 | 910473 | NM_025135 |
| | [Source: HGNC Symbol; Acc: 26178] | | | |
| AC007879.7:2524985 | | 2524983 | 129731 | ENST00000440326 |
| NKX3-1:3127989 | NK3 homeobox 1 [Source: HGNC | 3127978 | 506937 | NM_006167 |
| | Symbol; Acc: 7838] | | | |
| TRMT1:3852041 | TRM1 tRNA methyltransferase 1 | 3852034 | 950917 | NM_017722 |
| | homolog (S. cerevisiae) [Source: HGNC | | | |
| | Symbol; Acc: 25980] | | | |
| CHERP:3853971 | calcium homeostasis endoplasmic | 3853942 | 952004 | NM_006387 |
| | reticulum protein [Source: HGNC | | | |
| | Symbol; Acc: 16930] | | | |
| AC006504.1:3827591 | | 3827572 | 936884 | BC024732 |
| DEPDC1:2417549 | DEP domain containing 1 | 2417528 | 62894 | NM_001114120 |
| | [Source: HGNC Symbol; Acc: 22949] | | | |
| SHANK2:3380484 | SH3 and multiple ankyrin repeat | 3380365 | 662812 | AK095088 |
| | domains 2 [Source: HGNC | | | |
| | Symbol; Acc: 14295] | | | |
| RRP9:2675780 | ribosomal RNA processing 9, small | 2675763 | 224391 | NM_004704 |
| | subunit (SSU) processome component, | | | |
| | homolog (yeast) [Source: HGNC | | | |
| | Symbol; Acc: 16829] | | | |
| MOV10:2352284 | Mov10, Moloney leukemia virus 10, | 2352275 | 22984 | ENST00000369644 |
| | homolog (mouse) [Source: HGNC | | | |
| | Symbol; Acc: 7200] | | | |
| RRP9:2675766 | ribosomal RNA processing 9, small | 2675763 | 224384 | NM_004704 |
| | subunit (SSU) processome component, | | | |
| | homolog (yeast) [Source: HGNC | | | |
| | Symbol; Acc: 16829] | | | |
| PFAS:3709578 | phosphoribosylformylglycinamidine | 3709540 | 865047 | NM_012393 |
| | synthase [Source: HGNC | | | |
| | Symbol; Acc: 8863] | | | |
| TRMU:3949094 | tRNA 5-methylaminomethyl-2- | 3949055 | 1009051 | ENST00000160874 |
| | thiouridylate methyltransferase | | | |
| | [Source: HGNC Symbol; Acc: 25481] | | | |
| FHOD3:3784877 | formin homology 2 domain containing 3 | 3784840 | 910471 | NM_025135 |
| | [Source: HGNC Symbol; Acc: 26178] | | | |
| TIMM9:3566670 | translocase of inner mitochondrial | 3566652 | 777905 | NM_012460 |
| | membrane 9 homolog (yeast) | | | |
| | [Source: HGNC Symbol; Acc: 11819] | | | |
| PFAS:3709582 | phosphoribosylformylglycinamidine | 3709540 | 865047 | NM_012393 |
| | synthase [Source: HGNC | | | |
| | Symbol; Acc: 8863] | | | |
| THSD4:3600294 | thrombospondin, type I, domain | 3600283 | 798681 | NM_024817 |
| | containing 4 [Source: HGNC | | | |
| | Symbol; Acc: 25835] | | | |
| EEF1D:3157635 | eukaryotic translation elongation factor | 3157596 | 525707 | NM_001130057 |
| | 1 delta (guanine nucleotide exchange | | | |
| | protein) [Source: HGNC | | | |
| | Symbol; Acc: 3211] | | | |
| RP13-150K15.1:3993816 | | 3993810 | 1036121 | NM_017722 |
| PFAS:3709556 | phosphoribosylformylglycinamidine | 3709540 | 865031 | NM_012393 |
| | synthase [Source: HGNC | | | |
| | Symbol; Acc: 8863] | | | |
| AC012146.7:3707590 | | 3707584 | 863911 | AK056005 |
| B4GALNT1:3458723 | beta-1,4-N-acetyl-galactosaminyl | 3458700 | 710902 | NM_001478 |
| | transferase 1 [Source: HGNC | | | |
| | Symbol; Acc: 4117] | | | |
| GPBP1L1:2410386 | GC-rich promoter binding protein 1-like | 2410330 | 58348 | ENST00000488278 |
| | 1 [Source: HGNC Symbol; Acc: 28843] | | | |
| PFAS:3709546 | phosphoribosylformylglycinamidine | 3709540 | 865025 | NM_012393 |
| | synthase [Source: HGNC | | | |
| | Symbol; Acc: 8863] | | | |
| CCT4:2555668 | chaperonin containing TCP1, subunit 4 | 2555630 | 149087 | ENST00000461370 |
| | (delta) [Source: HGNC | | | |
| | Symbol; Acc: 1617] | | | |
| CD320:3848875 | CD320 molecule [Source: HGNC | 3848871 | 949104 | NM_016579 |
| | Symbol; Acc: 16692] | | | |
| MANF:2623152 | mesencephalic astrocyte-derived | 2623139 | 191523 | NM_006010 |
| | neurotrophic factor [Source: HGNC | | | |
| | Symbol; Acc: 15461] | | | |
| PFAS:3709583 | phosphoribosylformylglycinamidine | 3709540 | 865047 | NM_012393 |
| | synthase [Source: HGNC | | | |
| | Symbol; Acc: 8863] | | | |
| SEPT9:3735859 | septin 9 [Source: HGNC | 3735847 | 880922 | NM_006640 |
| | Symbol; Acc: 7323] | | | |
| AL590303.1:2971412 | | 2971403 | 408899 | AK125564 |
| CCDC99:2840013 | coiled-coil domain containing 99 | 2840002 | 327647 | ENST00000503871 |
| | [Source: HGNC Symbol; Acc: 26010] | | | |
| KHDC1:2960827 | KH homology domain containing 1 | 2960774 | 402249 | ENST00000398508 |
| | [Source: HGNC Symbol; Acc: 21366] | | | |
| AC012146.7:3707587 | | 3707584 | 863910 | AK056005 |
| PFAS:3709575 | phosphoribosylformylglycinamidine | 3709540 | 865047 | NM_012393 |
| | synthase [Source: HGNC | | | |
| | Symbol; Acc: 8863] | | | |
| UPP1:3000961 | uridine phosphorylase 1 [Source: HGNC | 3000953 | 427400 | NM_003364 |
| | Symbol; Acc: 12576] | | | |
| TRMU:3949093 | tRNA 5-methylaminomethyl-2- | 3949055 | 1009051 | ENST00000160874 |
| | thiouridylate methyltransferase | | | |
| | [Source: HGNC Symbol; Acc: 25481] | | | |
| RNF152:3811007 | ring finger protein 152 [Source: HGNC | 3811000 | 927110 | NM_173557 |
| | Symbol; Acc: 26811] | | | |
| PFAS:3709541 | phosphoribosylformylglycinamidine | 3709540 | 865021 | NM_012393 |
| | synthase [Source: HGNC | | | |
| | Symbol; Acc: 8863] | | | |
| SEPT9:3735857 | septin 9 [Source: HGNC | 3735847 | 880922 | NM_006640 |
| | Symbol; Acc: 7323] | | | |
| RP11-365D9.1:2386545 | | 2386541 | 43915 | ENST00000424229 |
| PRR3:2901679 | proline rich 3 [Source: HGNC | 2901660 | 365731 | NM_025263 |
| | Symbol; Acc: 21149] | | | |
| CD320:3848877 | CD320 molecule [Source: HGNC | 3848871 | 949105 | NM_016579 |
| | Symbol; Acc: 16692] | | | |

TABLE 4
Gene Isoform Set 4.

| Gene Isoform | | Transcript | | |
|---|---|---|---|---|
| (Gene:Probeset) | Description | Cluster Id | Exon ID | mRNA - Accession |
| VAMP5:2491684 | vesicle-associated membrane protein 5 | 2491676 | 108813 | NM_006634 |
| | (myobrevin) [Source: HGNC | | | |
| | Symbol; Acc: 12646] | | | |
| TNS1:2599224 | tensin 1 [Source: HGNC | 2599153 | 176537 | NM_022648 |
| | Symbol; Acc: 11973] | | | |
| SHANK2:3380379 | SH3 and multiple ankyrin repeat | 3380365 | 662737 | NM_012309 |
| | domains 2 [Source: HGNC | | | |
| | Symbol; Acc: 14295] | | | |
| SLC40A1:2591861 | solute carrier family 40 (iron-regulated | 2591837 | 171824 | NM_014585 |
| | transporter), member 1 [Source: HGNC | | | |
| | Symbol; Acc: 10909] | | | |
| SHANK2:3380374 | SH3 and multiple ankyrin repeat | 3380365 | 662735 | NM_012309 |
| | domains 2 [Source: HGNC | | | |
| | Symbol; Acc: 14295] | | | |
| THSD4:3600304 | thrombospondin, type I, domain | 3600283 | 798689 | NM_024817 |
| | containing 4 [Source: HGNC | | | |
| | Symbol; Acc: 25835] | | | |
| HIST2H2BE:2434126 | histone cluster 2, H2be [Source: HGNC | 2434124 | 73057 | NM_003528 |
| | Symbol; Acc: 4760] | | | |
| TAF1B:2469139 | TATA box binding protein (TBP)- | 2469094 | 94444 | NM_005680 |
| | associated factor, RNA polymerase I, | | | |
| | B, 63 kDa [Source: HGNC | | | |
| | Symbol; Acc: 11533] | | | |
| CAMK2N1:2400179 | calcium/calmodulin-dependent protein | 2400177 | 52108 | NM_018584 |
| | kinase II inhibitor 1 [Source: HGNC | | | |
| | Symbol; Acc: 24190] | | | |
| THSD4:3600289 | thrombospondin, type I, domain | 3600283 | 798677 | NM_024817 |
| | containing 4 [Source: HGNC | | | |
| | Symbol; Acc: 25835] | | | |
| SLC40A1:2591875 | solute carrier family 40 (iron-regulated | 2591837 | 171831 | NM_014585 |
| | transporter), member 1 [Source: HGNC | | | |
| | Symbol; Acc: 10909] | | | |
| CENPV:3747208 | centromere protein V [Source: HGNC | 3747199 | 887780 | ENST00000476243 |
| | Symbol; Acc: 29920] | | | |
| CENPV:3747216 | centromere protein V [Source: HGNC | 3747199 | 887784 | NM_181716 |
| | Symbol; Acc: 29920] | | | |
| TNS1:2599214 | tensin 1 [Source: HGNC | 2599153 | 176530 | NM_022648 |
| | Symbol; Acc: 11973] | | | |
| PLXNA4:3073313 | plexin A4 [Source: HGNC | 3073267 | 472384 | NM_020911 |
| | Symbol; Acc: 9102] | | | |
| OCLN:2813603 | occludin [Source: HGNC | 2813593 | 311296 | NM_002538 |
| | Symbol; Acc: 8104] | | | |
| SLC40A1:2591889 | solute carrier family 40 (iron-regulated | 2591837 | 171841 | NM_014585 |
| | transporter), member 1 [Source: HGNC | | | |
| | Symbol; Acc: 10909] | | | |
| PAQR3:2774871 | progestin and adipoQ receptor family | 2774870 | 286616 | ENST00000512733 |
| | member III [Source: HGNC | | | |
| | Symbol; Acc: 30130] | | | |
| HSD17B2:3671095 | hydroxysteroid (17-beta) | 3671076 | 842057 | NM_002153 |
| | dehydrogenase 2 [Source: HGNC | | | |
| | Symbol; Acc: 5211] | | | |
| ITGA3:3726188 | integrin, alpha 3 (antigen CD49C, | 3726154 | 874988 | NM_002204 |
| | alpha 3 subunit of VLA-3 receptor) | | | |
| | [Source: HGNC Symbol; Acc: 6139] | | | |
| DHX33:3742750 | DEAH (Asp-Glu-Ala-His) box | 3742727 | 885077 | NM_020162 |
| | polypeptide 33 [Source: HGNC | | | |
| | Symbol; Acc: 16718] | | | |
| EFS:3557411 | embryonal Fyn-associated substrate | 3557408 | 772276 | NM_005864 |
| | [Source: HGNC Symbol; Acc: 16898] | | | |
| ITGA3:3726180 | integrin, alpha 3 (antigen CD49C, | 3726154 | 874981 | NM_002204 |
| | alpha 3 subunit of VLA-3 receptor) | | | |
| | [Source: HGNC Symbol; Acc: 6139] | | | |
| TNS1:2599212 | tensin 1 [Source: HGNC | 2599153 | 176529 | NM_022648 |
| | Symbol; Acc: 11973] | | | |
| THSD4:3600307 | thrombospondin, type I, domain | 3600283 | 798691 | NM_024817 |
| | containing 4 [Source: HGNC | | | |
| | Symbol; Acc: 25835] | | | |
| APOD:4054213 | apolipoprotein D [Source: HGNC | 4054204 | 1072341 | NM_001647 |
| | Symbol; Acc: 612] | | | |
| ITGA3:3726161 | integrin, alpha 3 (antigen CD49C, | 3726154 | 874967 | NM_002204 |
| | alpha 3 subunit of VLA-3 receptor) | | | |
| | [Source: HGNC Symbol; Acc: 6139] | | | |
| TNPO2:3851696 | transportin 2 [Source: HGNC | 3851651 | 950729 | NM_013433 |
| | Symbol; Acc: 19998] | | | |
| TNS1:2599225 | tensin 1 [Source: HGNC | 2599153 | 176538 | NM_022648 |
| | Symbol; Acc: 11973] | | | |
| SLC40A1:2591877 | solute carrier family 40 (iron-regulated | 2591837 | 171832 | NM_014585 |
| | transporter), member 1 [Source: HGNC | | | |
| | Symbol; Acc: 10909] | | | |
| ABAT:3647484 | 4-aminobutyrate aminotransferase | 3647421 | 827803 | NM_020686 |
| | [Source: HGNC Symbol; Acc: 23] | | | |
| ITGA3:3726203 | integrin, alpha 3 (antigen CD49C, | 3726154 | 874997 | NM_002204 |
| | alpha 3 subunit of VLA-3 receptor) | | | |
| | [Source: HGNC Symbol; Acc: 6139] | | | |
| ITGA3:3726190 | integrin, alpha 3 (antigen CD49C, | 3726154 | 874990 | ENST00000504417 |
| | alpha 3 subunit of VLA-3 receptor) | | | |
| | [Source: HGNC Symbol; Acc: 6139] | | | |
| ITGA3:3726199 | integrin, alpha 3 (antigen CD49C, | 3726154 | 874997 | NM_002204 |
| | alpha 3 subunit of VLA-3 receptor) | | | |
| | [Source: HGNC Symbol; Acc: 6139] | | | |
| THSD4:3600339 | thrombospondin, type I, domain | 3600283 | 798717 | NM_024817 |
| | containing 4 [Source: HGNC | | | |
| | Symbol; Acc: 25835] | | | |
| TNS 1:2599220 | tensin 1 [Source: HGNC | 2599153 | 176535 | NM_022648 |
| | Symbol; Acc: 11973] | | | |
| TRMT1:3852045 | TRM1 tRNA methyltransferase 1 | 3852034 | 950918 | NM_017722 |
| | homolog (S. cerevisiae) [Source: HGNC | | | |
| | Symbol; Acc: 25980] | | | |
| C16orf7:3704944 | chromosome 16 open reading frame 7 | 3704939 | 862422 | NM_004913 |
| | [Source: HGNC Symbol; Acc: 13526] | | | |
| ITGA3:3726169 | integrin, alpha 3 (antigen CD49C, | 3726154 | 874973 | ENST00000505552 |
| | alpha 3 subunit of VLA-3 receptor) | | | |
| | [Source: HGNC Symbol; Acc: 6139] | | | |
| ADCY6:3453265 | adenylate cyclase 6 [Source: HGNC | 3453252 | 707801 | NM_015270 |
| | Symbol; Acc: 237] | | | |
| FAM161A:2555617 | family with sequence similarity 161, | 2555604 | 149057 | NM_032180 |
| | member A [Source: HGNC | | | |
| | Symbol; Acc: 25808] | | | |
| FAM65C:3909291 | family with sequence similarity 65, | 3909247 | 984917 | AK295781 |
| | member C [Source: HGNC | | | |
| | Symbol; Acc: 16168] | | | |
| TNS1:2599250 | tensin 1 [Source: HGNC | 2599153 | 176556 | NM_022648 |
| | Symbol; Acc: 11973] | | | |
| ITGA3:3726179 | integrin, alpha 3 (antigen CD49C, | 3726154 | 874980 | NM_002204 |
| | alpha 3 subunit of VLA-3 receptor) | | | |
| | [Source: HGNC Symbol; Acc: 6139] | | | |
| FAM49A:2541718 | family with sequence similarity 49, | 2541699 | 140179 | NM_030797 |
| | member A [Source: HGNC | | | |
| | Symbol; Acc: 25373] | | | |
| DNER:2602804 | delta/notch-like EGF repeat containing | 2602770 | 178855 | NM_139072 |
| | [Source: HGNC Symbol; Acc: 24456] | | | |
| ITGA3:3726162 | integrin, alpha 3 (antigen CD49C, | 3726154 | 874967 | NM_002204 |
| | alpha 3 subunit of VLA-3 receptor) | | | |
| | [Source: HGNC Symbol; Acc: 6139] | | | |

TABLE 5
Gene Isoform Set 5.

| Gene Isoform | | Transcript | | |
|---|---|---|---|---|
| (Gene:Probeset) | Description | Cluster Id | Exon ID | mRNA - Accession |
| TBC1D30:3419983 | TBC1 domain family, member 30 | 3419969 | 687144 | — |
| IGF2BP3:3041430 | ENSG00000136231 | 3041409 | 452513 | NM_006547 |
| CDH11:3694727 | ENSG00000140937 | 3694657 | 856198 | NM_001797 |
| AP1S2:4000708 | ENSG00000182287 | 4000704 | 1040261 | NM_003916 |
| NNMT:3349874 | ENSG00000166741 | 3349858 | 644518 | NM_006169 |
| LPAR1:3220416 | ENSG00000198121 | 3220384 | 564156 | NM_001401 |
| CMTM3:3664867 | ENSG00000140931 | 3664843 | 838217 | NM_144601 |
| SLC9A3R1:3734455 | ENSG00000109062 | 3734453 | 880133 | NM_004252 |
| MYO18A:3751344 | ENSG00000196535 | 3751323 | 890128 | NM_078471 |
| ABI3BP:2686553 | ENSG00000154175 | 2686458 | 231398 | NM_015429 |
| GPR160:2651853 | G protein-coupled receptor 160 | 2651835 | 209551 | ENST00000482813 |
| ZEB2:2579575 | ENSG00000169554 | 2579572 | 163895 | NM_014795 |
| PREX1:3908647 | ENSG00000124126 | 3908631 | 984493 | ENST00000396220 |
| ZEB2:2579584 | ENSG00000169554 | 2579572 | 163900 | NM_014795 |
| COL8A1:2633418 | ENSG00000144810 | 2633390 | 197890 | AF170702 |
| NRP2:2524318 | ENSG00000118257 | 2524301 | 129329 | NM_201266 |
| ANK3:3290920 | ENSG00000151150 | 3290875 | 608308 | NM_020987 |
| SEPP1:2855307 | ENSG00000250722 | 2855285 | 337262 | NM_001093726 |
| CMTM3:3664861 | ENSG00000140931 | 3664843 | 838214 | NM_144601 |
| SLC40A1:2591894 | ENSG00000138449 | 2591837 | 171845 | ENST00000427241 |
| FGF5:2733387 | ENSG00000138675 | 2733360 | 260582 | NM_004464 |
| CACNA1D:2624455 | ENSG00000157388 | 2624385 | 192274 | NM_000720 |
| COL6A1:3924402 | ENSG00000142156 | 3924372 | 994306 | NM_001848 |
| CAV2:3020292 | ENSG00000105971 | 3020273 | 439314 | NM_001233 |
| C17orf28:3770528 | chromosome 17 open reading frame 28 | 3770512 | 901753 | — |
| S100A14:4045674 | ENSG00000189334 | 4045665 | 1067382 | ENST00000368702 |
| COL6A1:3924415 | ENSG00000142156 | 3924372 | 994314 | NM_001848 |
| FHL1:3992417 | ENSG00000022267 | 3992408 | 1035268 | NR_027621 |
| C17orf28:3770521 | ENSG00000167861 | 3770512 | 901749 | AK125514 |
| MXRA7:3771753 | ENSG00000182534 | 3771744 | 902455 | NM_001008529 |
| DDAH1:2420905 | ENSG00000153904 | 2420832 | 64979 | NM_001134445 |
| LOXL2:3127862 | ENSG00000134013 | 3127818 | 506856 | NM_002318 |
| COL4A1:3525330 | ENSG00000187498 | 3525313 | 752675 | NM_001845 |
| FRMD4A:3278517 | ENSG00000151474 | 3278401 | 600461 | NM_018027 |
| SYCP2:3912136 | ENSG00000196074 | 3912079 | 986680 | ENST00000357552 |
| RUNX1:3930506 | ENSG00000159216 | 3930360 | 998038 | NM_001754 |

TABLE 6
Gene Isoform Set 6.

| Gene Isoform | | Transcript | | |
|---|---|---|---|---|
| (Gene:Probeset) | Description | Cluster Id | Exon ID | mRNA - Accession |
| ALDH3B2:3379104 | ENSG00000132746 | 3379091 | 661951 | NM_000695 |
| EPN3:3726547 | ENSG00000049283 | 3726537 | 875204 | NM_017957 |
| BLNK:3301732 | ENSG00000095585 | 3301713 | 615115 | NM_013314 |
| SLK:3262461 | ENSG00000065613 | 3262433 | 590321 | NM_014720 |
| SLIT2:2720663 | ENSG00000145147 | 2720584 | 252613 | ENST00000511508 |
| SELENBP1:2435018 | ENSG00000143416 | 2435005 | 73589 | NM_003944 |
| SYT14:2378266 | ENSG00000143469 | 2378256 | 38871 | NM_001146261 |
| LPAR1:3220437 | lysophosphatidic acid receptor 1 | 3220384 | 564176 | — |
| CAV2:3020233 | caveolin 2 | 3020226 | 439281 | ENST00000490906 |
| DSE:2922649 | ENSG00000111817 | 2922631 | 378615 | NM_013352 |
| EPS8L1:3841962 | ENSG00000131037 | 3841949 | 945192 | NM_133180 |
| ENAH:2458376 | ENSG00000154380 | 2458338 | 87633 | NM_001008493 |
| CAV2:3020274 | caveolin 2 | 3020273 | 439306 | ENST00000477018 |
| SEPP1:2855296 | ENSG00000250722 | 2855285 | 337256 | NM_005410 |
| LPAR1:3220435 | ENSG00000198121 | 3220384 | 564174 | NM_001401 |
| IGF2BP3:3041433 | ENSG00000136231 | 3041409 | 452514 | ENST00000435131 |
| CALD1:3025633 | ENSG00000122786 | 3025545 | 442755 | NM_033138 |
| DOCK10:2601665 | ENSG00000135905 | 2601648 | 178092 | NM_014689 |
| ZNF655:3014906 | ENSG00000197343 | 3014904 | 436055 | NM_138494 |
| IL6:2992593 | ENSG00000136244 | 2992576 | 422093 | AK298013 |
| HSPB1:3009411 | heat shock 27 kDa protein 1 | 3009399 | 432552 | — |
| SGK1:2975060 | serum/glucocorticoid regulated kinase1 | 2975014 | 411240 | — |
| CD109:2913758 | ENSG00000156535 | 2913694 | 373011 | NM_133493 |
| RP11-429J17.6:3119845 | ENSG00000203499 | 3119826 | 501803 | AK125852 |
| CDH11:3694702 | ENSG00000140937 | 3694657 | 856183 | NM_001797 |
| NAV2:3323176 | ENSG00000166833 | 3323052 | 628409 | NM_001111019 |
| ABCC4:3521306 | ENSG00000125257 | 3521174 | 750204 | AY133679 |
| ABCC4:3521225 | ENSG00000125257 | 3521174 | 750140 | NM_001105515 |
| RAB17:2605498 | ENSG00000124839 | 2605480 | 180506 | NM_022449 |
| NAV2:3323175 | ENSG00000166833 | 3323052 | 628409 | AK298346 |
| DDR2:2364253 | ENSG00000162733 | 2364231 | 29887 | NM_001014796 |
| EPB41L2:2974081 | ENSG00000079819 | 2973995 | 410642 | ENST00000368128 |

TABLE 8
Gene Isoform Set 8.
Gene Name *** See Tables 1-6 for gene isoform disclosure

| | |
|---|---|
| AC007276 | GPBP1L1 |
| ANXA9 | GRHL1 |
| ARHGAP8 | HRH1 |
| ATP2C2_e1 | IGF2BP3 |
| ATP2C2_e2 | IL6 |
| C17orf28 | IRF6 |
| CACHA1D | KIAA1543 |
| CALD1 | MARK3 |
| CAPN13 | MRC2 |
| CAV1 | MUC1 |
| CCDC99 | MXRA7 |
| CLSTN1 | MYO18A |
| COL4A1 | NUS1 |
| CYBASC3 | NRP2 |
| DDR2 | PRKCDBP |
| DNMT3B | PSD4 |
| ENAH | RFX2 |
| EPN3_e1 | RP11-365D9 |
| EPN3_e2 | RP11-429J17 |
| EPN3_e3 | RUNX1 |
| EPS8L1 | SELENBP1 |
| ESRP2 | SLK |
| FGF5 | SULT1A1 |
| FIP1 | SULT2B1 |
| FLNB FNIP1 | SYCP2 |
| VPS39 | S100A14 |
| TRMU | |

TABLE 9
Gene Isoform Set 9.
Gene Name *** See Tables 1-6 for gene isoform disclosure

ATP2C2
CYBASC3
EPN3
HRH1
PRKCDBP
SULT2B1
SYCP2
GRHL1
PSD4
C17orf28
DNMT3B
FNIP1
DDR2
MARK3
RUNX1

TABLE 10
Gene Isoform Set 10.
Gene Name *** See Tables 1-6 for gene isoform disclosure
ATP2C2

EPN3
SULT2B3
SYCP2
GRHL1
PSD4
SULT1A1
DNMT3B
FNIP1
DDR2
MARK3

TABLE 11
Gene Isoform Set 11.
Gene Name *** See Tables 1-6 for gene isoform disclosure

AC007276
ANXA9
ATP2C2_e1
ATP2C2_e2
C17orf8
CAPN13
CAV1
CLSTN1
COL4A1
ENAH
FNIP1
IGF2BP3
IL6
MRC2
MYO18A
RFX2
RP11-429J17
SLK
TRMU
VPS39
DNMT3B
KIAA1543
MARK3
RP11-365D9

TABLE 12
Gene Isoform Set 12.
Gene Name *** See Tables 1-6 for gene isoform disclosure

FGFR2_e1
FLNB
PPFIBP1
MUC1
DTNB
SLC37A2

TABLE 13
Gene Isoform Set 13.
Gene Name *** See Tables 1-6 for gene isoform disclosure

FGFR2_e1,
MUC1,
FLNB,
SLC37A2

In an embodiment, said plurality of gene isoforms is elected from gene isoform set one, two, three, four, five, six, seven, eight, nine, ten, eleven, twelve, and/or thirteen. In an embodiment, said plurality of gene isoforms is elected from gene isoform set one. In an embodiment, said plurality of gene isoforms is elected from gene isoform set two. In an embodiment, said plurality of gene isoforms is elected from gene isoform set three. In an embodiment, said plurality of gene isoforms is elected from gene isoform set four. In an embodiment, said plurality of gene isoforms is elected from gene isoform set five. In an embodiment, said plurality of gene isoforms is elected from gene isoform set six. In an embodiment, said plurality of gene isoforms is elected from gene isoform set seven. In an embodiment, said plurality of gene isoforms is elected from gene isoform set eight. In an embodiment, said plurality of gene isoforms is elected from gene isoform set nine. In an embodiment, said plurality of gene isoforms is elected from gene isoform set ten. In an embodiment, said plurality of gene isoforms is elected from gene isoform set eleven. In an embodiment, said plurality of gene isoforms is elected from gene isoform set twelve. In an embodiment, said plurality of gene isoforms is elected from gene isoform set thirteen.

In an embodiment, said plurality of gene isoforms comprises at least two gene isoforms; four gene isoforms; six gene isoforms; eight gene isoforms; ten gene isoforms; twelve gene isoforms; fourteen gene isoforms; sixteen gene isoforms; eighteen gene isoforms; twenty gene isoforms; twenty five gene isoforms; thirty gene isoforms; forty gene isoforms; or any range intervening there between. In an embodiment, said plurality comprises more than forty gene isoforms.

In an embodiment, said plurality of gene isoforms comprises or consists of a first gene isoform. In an embodiment, said plurality of gene isoforms comprises or consists of, a first gene isoform and a second gene isoform. In an embodiment, said plurality of gene isoforms further comprises, or consists of, a third gene isoform; a third and fourth gene isoform; a third, fourth, and fifth gene isoform; a third, fourth, fifth, and sixth gene isoform; a third, fourth, fifth, sixth, and seventh gene isoform; a third, fourth, fifth, sixth, seventh, and eighth gene isoform; a third, fourth, fifth, sixth, seventh, eighth and ninth gene isoform; a third, fourth, fifth, sixth, seventh, eighth, ninth, and tenth gene isoform. In an embodiment, said plurality of gene isoforms comprises of more than ten gene isoforms.

In an embodiment, said value or values is a function of the level of expression of a first gene isoform and the level of expression of a second gene isoform. In an embodiment, said value or values is a function of the level of expression of a gene isoform of said first, second, and a third gene isoform; a third and fourth gene isoform; a third, fourth, and fifth gene isoform; a third, fourth, fifth, and sixth gene isoform; a third, fourth, fifth, sixth, and seventh gene isoform; a third, fourth, fifth, sixth, seventh, and eighth gene isoform; a third, fourth, fifth, sixth, seventh, eighth and ninth gene isoform; a third, fourth, fifth, sixth, seventh, eighth, ninth, and tenth gene isoform. In an embodiment, said value or values is a function of the level of expression of a gene isoform of more than ten gene isoform s.

In an embodiment, a first value that is a function of the level of expression of said first gene and a second value that is a function of the level of expression of said second gene isoform are acquired. In an embodiment, a first value that is a function of the level of expression of said first gene isoform, a second value that is a function of the level of expression of said second gene isoform, a third value that is a function of the level of expression of said third gene isoform, a fourth value that is a function of the level of expression of said fourth gene isoform, a fifth value that is a function of the level of expression of said fifth gene isoform, a sixth value that is a function of the level of expression of said sixth gene isoform, a seventh value that is a function of the level of expression of said seventh gene isoform, a eighth value that is a function of the level of expression of said eighth gene isoform, a ninth value that is a function of the level of expression of said ninth gene isoform, and a tenth value that is a function of the level of expression of said tenth gene isoform is acquired. In an embodiment, a plurality of values that is each a function of the level of expression of a plurality of gene isoforms is acquired. In an embodiment, more than ten values that is each a function of the level of expression of a plurality of gene isoforms is acquired.

In an embodiment, a first value that is a function of the level of expression of two or more gene isoforms of said plurality of gene isoforms and a second value that is a function of the level of expression of one of the gene isoforms of the plurality are acquired. In an embodiment, the invention further features the acquisition of a value or values that is a function of the level of expression of a gene isoform not in said first, second, third, fourth, fifth, sixth, seventh, eighth, ninth, tenth, eleventh, twelfth, or thirteenth gene isoform sets. In an embodiment, the invention further features the acquisition of a plurality of value or values that is a function of the level of expression of a plurality of gene isoforms not in said first, second, third, fourth, fifth, sixth, seventh, eighth, ninth, tenth, eleventh, twelfth, or thirteenth gene isoform sets.

In an embodiment, the invention features the acquisition of a value, e.g., a composite value that is a function of the level of expression of said first gene isoform, the level of expression of said second gene isoform, and a weighting factor. In an embodiment, one of said first value or said second value is a function of a weighting factor. In an embodiment, said first value is a function of a first weighting factor and said second value is a function of a second weighting factor. In an embodiment, said first weighting factor and said second weighting factor are different. In an embodiment, the invention features the acquisition of a value, e.g., a composite value, which is a function of the level of expression of each of a plurality of gene isoforms, and a weighting factor. In an embodiment, the value of the level of expression of each gene isoform in said plurality of gene isoforms is a function of a weighting factor. In an embodiment, the value of the level of expression of each gene isoform in said plurality of genes is a function of a different weighting factor.

In an embodiment, said plurality of genes comprises or consists of, a first gene isoform of a first gene. In an embodiment, the invention features the acquisition of a value that is the function of the level of expression of said first gene isoform of said first gene. In an embodiment, the invention features the acquisition of a value that is a function of the level of expression of said first gene isoform of said first gene and a second gene isoform of said first gene. In an embodiment, the invention features the acquisition of a first value that is a function of the level of expression of said first gene isoform of said first gene and a second value that is a function of a second gene isoform of said first gene. In an embodiment, said plurality of gene isoforms further comprises, or consists of, a third gene isoform of said first gene; a third and fourth gene isoform of said first gene; a third, fourth, and fifth gene isoform of said first gene; a third, fourth, fifth, and sixth gene isoform of said first gene; a third, fourth, fifth, sixth, and seventh gene isoform of said first gene; a third, fourth, fifth, sixth, seventh, and eighth gene isoform of said first gene; a third, fourth, fifth, sixth, seventh, eighth and ninth gene isoform of said first gene; a third, fourth, fifth, sixth, seventh, eighth, ninth, and tenth gene isoform of said first gene. In an embodiment, said plurality of gene isoforms comprises of more than ten gene isoforms of said first gene.

In an embodiment, the invention features the acquisition of a first value that is a function of the level of expression of a first gene isoform of a first gene, a second value that is a function of the level of expression of a second gene isoform of said first gene, a third value that is a function of the level of expression of a third gene isoform of said first gene, a fourth value that is a function of the level of expression of a fourth gene isoform of said first gene, a fifth value that is a function of the level of expression of a fifth gene isoform of said first gene, a sixth value that is a function of the level of expression of a sixth gene isoform of said first gene, a seventh value that is a function of the level of expression of a seventh gene isoform of said seventh gene, an eighth value that is a function of the level of expression of an eighth gene isoform of said first gene, a ninth value that is a function of the level of expression of a ninth gene isoform of said first gene, and a tenth value that is a function of the level of expression of a tenth gene isoform of said first gene.

In an embodiment, the invention features the acquisition of a first value that is a function of the level of expression of two or more gene isoforms of a first gene and a second value that is a function of the level of expression of a gene isoform of said first gene. In an embodiment, the invention features the acquisition of a value that is a function of the level of expression of a first gene isoform of said first gene, the level of expression of a second gene isoform of said first gene, and a weighting factor. In an embodiment, one of said first value or said second value is a function of a weighting factor. In an embodiment, said first value is a function of a first weighting factor and said second value is a function of a second weighting factor. In an embodiment, said first weighting factor and said second weighting factor are different. In an embodiment, said value or values is a function of a comparison with a reference criterion. In an embodiment, said value or values is further a function of the determination of whether the level of expression of a gene isoform has a preselected relationship with a reference criterion. In an embodiment, said value or values is a function of said determination.

In an embodiment, the invention features the acquisition of a value or values that is a function of the level of expression of a plurality of gene isoforms that is further a function of a comparison with a reference criterion. In an embodiment, said value or values is a function of the determination of whether the level of expression of a gene isoform has a preselected relationship with a reference criterion, e.g., comparing said level of expression, with a preselected reference. In an embodiment, said value or values is a function of said determination. In an embodiment, the invention features determining if said value or values has a preselected relationship with a reference criterion. In an embodiment, the invention features the acquisition of said value or values at a predetermined interval, e.g., a first point in time and at least a subsequent point in time.

In an embodiment, the invention features the acquisition of a value or values that is a function of the level of expression of a gene isoform of a gene. In an embodiment, the invention features the acquisition of a values or values that is a function of the level expression of each gene isoform of a plurality of gene isoforms of a gene. In an embodiment, the invention features the acquisition of a values or values that is a function of the level of expression of a plurality of gene isoforms of a gene. In an embodiment, the invention features the acquisition of a values or values that is a function of the level of expression of each gene isoform of a plurality of gene isoforms of a plurality of genes. In an embodiment, the invention features the acquisition of a values or values that is a function of the level of expression of a plurality of gene isoforms of a plurality of genes. In an embodiment, the level of expression of said gene isoform or said plurality of gene isoforms is a function of the level of expression of an alternatively spliced exon of said gene isoform or a plurality of alternatively spliced exons of said gene isoforms. In an embodiment, said gene or said plurality of genes is in gene isoform set 1, gene isoform set 2, gene isoform set 3, gene isoform set 4, gene isoform set 5, gene isoform set 6, gene isoform set 7, gene isoform set 8, gene isoform set 9, gene isoform set 10, gene isoform set 11, gene isoform set 12, and/or gene isoform set 13.

In an embodiment, the invention features the further acquisition of a value that is a function of the level of gene expression of a gene. In an embodiment, the invention features the acquisition of a value that is the function of the level of gene expression of a plurality of genes. In an embodiment, the invention features the acquisition of a value that is a function of the level of gene expression of each gene of a plurality of genes. In an embodiment, the level of gene expression is a function of the level of RNA expression of said gene or plurality of genes. In an embodiment, the level of gene expression is a function of the level of protein expression of said gene or plurality of genes. In an embodiment, said gene or plurality of genes is in Table 7.

### Gene Set Score

In an embodiment, the invention features the acquisition of a gene set score. In an embodiment, the gene set score is a function of a value or values that is a function of the level of gene expression of said plurality of genes in said gene isoform sets one and/or two and/or three and/or four and/or five and/or six and/or seven and/or eight and/or nine and/or ten and/or eleven and/or twelve and/or thirteen. In an embodiment, the gene set score is a function of a value or values that is a function of the level of gene expression of said plurality of genes in said gene isoform sets one and/or two and/or three and/or four and/or five and/or six and/or seven and/or eight and/or nine and/or ten and/or eleven and/or twelve and/or thirteen and further a function of the level of gene expression of a gene or plurality of genes in Table 7.

TABLE 7
Genes of tumor initiation, EMT, and Cancer Stem Cell classifiers

| | | | | | |
|---|---|---|---|---|---|
| DPF2 | KIAA0436 | CLTC | RAD51L1 | STAU1 | CTSL2 |
| CASP8 | CYP4V2 | COPB2 | EPPK1 | TUBB3 | CXADR |
| BCL2 | JTV1 | SLC25A25 | COL1A1 | UBE2S | CYP27B1 |
| SCGN | ICMT | ECOP | MMP9 | XPNPEP1 | DSC2 |
| SWAP70 | DNMT3A | PDE8A | SERPINE1 | CDKN1A | DSG3 |
| KIAA0276 | HNMT | STAM | SPARC | CHRD | DST |
| C10orf9 | METTL7A | TUBB | TGFB1 | H19 | EPB41L4B |
| C10orf7 | METTL2 | SNX6 | TGFB3 | ID3 | FGFBP1 |
| ALKBH | VIL2 | RAB23 | TGFBI | ID4 | FGFR3 |
| TOB2 | TPD52 | PLAA | TGFBR1 | IGFBP7 | FST |
| XPR1 | ARPC5 | STC2 | TGIF | LRP1 | GJB3 |
| CD59 | NOL8 | LTF | TGIF2 | MSX1 | GRHL2 |
| LRP2 | NSF | ISGF3G | THBS1 | NOTCH3 | HBEGF |
| PLP2 | RAD23B | ATXN3 | ANXA5 | PROCR | HOOK1 |
| MAPK14 | SRP54 | GTF3C3 | ACTG1 | GBX2 | IL18 |
| CXCL2 | HSPA2 | GSK3B | ARF3 | KI67 | IL1B |
| MMP7 | PBP | KLF10 | ATP1B3 | CCNB1 | IRF6 |
| MGP | THAP2 | ELL2 | BAT3 | BUB1 | ITGB4 |
| MLF1 | CIRBP | ZBTB20 | CALD1 | KNTC2 | JAG2 |
| FLNB | SNRPN | IRX3 | CENTD2 | USP22 | KLK10 |
| SCNM1 | KIAA0052 | ETS1 | CLIC1 | HCFC1 | KLK5 |
| HSPC163 | DUSP10 | SERTAD1 | CTBS | RNF2 | KLK7 |
| CSorf18 | SSR1 | MGC4251 | DPYSL3 | ANK3 | KLK8 |
| MGC4399 | ERBB4 | MAFF | DVL3 | FGFR2 | KRT15 |
| CDW92 | EMP1 | SFPQ | EXT1 | CES1 | KRT16 |
| TMC4 | CHPT1 | CITED4 | FGFR1 | COL1A2 | KRT17 |
| ZDHHC2 | LRPAP1 | CEBPD | FTL | COL3A1 | LEPREL1 |
| TICAM2 | FLJ11752 | EIF4E2 | GNB2L1 | COL5A2 | MYO5C |
| KDELR3 | CSTF1 | HS2ST1 | GPRC5A | COL6A1 | NDRG1 |
| GNPDA1 | KLHL20 | AGPS | H2AFZ | ANKRD25 | NMU |
| THEM2 | DNAJC13 | PGK1 | HIF1A | C10ORF56 | PI3 |
| DBR1 | APLP2 | ATIC | IL13RA1 | C5ORF13 | RAB25 |
| FLJ90709 | ARGBP2 | ETNK1 | KDELR2 | KRT81 | RLN2 |
| FLJ10774 | DNAJB1 | LG2 | LARP1 | N-PAC | RNF128 |
| C16orf33 | NEBL | NCE2 | LPIN2 | PLEKHC1 | S100A14 |
| GAPD | SH3BGRL | 8-Mar | MARS | 9-Sep | S100A7 |
| LDHA | NUDT5 | CNOT4 | MMP10 | SYNC1 | S100A8 |
| MR-1 | GABARAPL1 | RNF8 | MMP14 | MBP | SERPINB1 |
| LARS | MAPT | PSMA5 | MT2A | ABLIM1 | SERPINB2 |
| GTPBP1 | DCBLD1 | DPF2 | MYO10 | ALDH1A3 | SLC2A9 |
| PRSS16 | STK39 | AMMECR1 | NUP62 | ALOX15B | SLPI |
| WFDC2 | PAK2 | KIAA1287 | ROR1 | TUBA1A | ESRP1 |
| AIM1 | CSNK2A1 | LOC144233 | DLC1 | PPM1D | CLDN3 |
| DHRS6 | PILRB | LOC286505 | GNG11 | TWIST1 | CLDN4 |
| DHRS4 | ERN1 | PNAS-4 | CDH11 | FN1 | ERBB3 |
| GC15429 | SGKL | FLJ20530 | NR2F1 | TGFBR3 | SPOCK1 |
| MGC45840 | WEE1 | HUMPD3 | PRR16 | SERPINF1 | FERMT2 |
| ECHDC2 | MAST4 | GC45564 | MYL9 | UGDH | GLYR1 |
| GOLGIN-67 | C11orf17 | CAP350 | DOCK10 | SRGN | LTBP1 |
| AFURS1 | NUP37 | ETAA16 | LRIG1 | FAP | FADS2 |
| HAN11 | GAS7 | ZNF335 | IER3 | PTGER4 | KANK2 |
| DNAPTP6 | TRAM2 | SH3KBP1 | EML1 | PRKCA | PTGFR |
| C7orf25 | BASP1 | MST150 | NEBL | FSTL1 | COL11A2 |
| FLJ37953 | FOXO1A | PRO 1073 | RGL1 | MMP1 | KLK3 |
| FLJ10587 | POLR2A | LOC388397 | MLPH | NRP1 | EIF2C2 |
| C7orf36 | PER1 | FKBP5 | DNAJB4 | FILIP1L | ZFP41 |
| ELP4 | DDIT4 | HIPK2 | FBLN5 | SCCPDH | FAM49B |
| NDEL1 | CD97 | KLF13 | RGS4 | LTBP2 | PSORS1C2 |
| NPD014 | BIN1 | ANTXR2 | HAS2 | XYLT1 | MRPL42 |
| KFZP564D172 | SH2B3 | IFNAR1 | ITGBL1 | HS3ST2 | MRPL54 |
| FAM53C | DDB2 | LIX1L | IGFBP4 | SYT11 | MRPL47 |
| IER5 | EMP3 | CHST11 | DPT | TSHZ1 | MRPS23 |
| LOC255783 | NDST2 | AKAP2 | PCOLCE | THY1 | EIF3S9 |
| KIAA0146 | CHST2 | DTX1 | GREM1 | 9-Sep | ALG5 |
| KIAA0792 | NT5E | ST3GAL2 | PPAP2B | S100A4 | DNAJC19 |
| LOC439994 | PDE4A | ADAMTS7 | CDH2 | TNS3 | TPRXL |
| LOC283481 | CPS1 | TNRC6B | PMP22 | ENOX1 | NOTCH2 |
| CG018 | PTGS1 | CYGB | LUM | TGFB1I1 | RBM15 |
| LOC130576 | GGCX | SDHAL1 | CHN1 | ZEB2 | ST3GAL3 |
| NGFRAP1L1 | IRF5 | LOC572558 | CYP1B1 | LMCD1 | NFYA |
| KIAA1217 | ZBTB16 | TRIO | MME | PDGFC | PCNX |
| 4orf7 | MAP4K4 | FRAS1 | WNT5A | ECM1 | FBXO21 |
| C21orf86 | CHST7 | KIAA1632 | POSTN | TFPI | WWOX |
| C9orf64 | KLF12 | POLS | MMP2 | TBX3 | CAMK2B |
| FLJ13456 | NFRKB | EBF | CTGF | DDR2 | PNPLA2 |
| KIAA1600 | PSD | MAML2 | CLIC5 | PFKFB3 | ANXA3 |
| B7-H4 | FKSG49 | PTPRA | UGCGL1 | PLOD2 | AP1M2 |
| LOC80298 | NIFUN | PLEKHG2 | FBXL18 | PSMB7 | ARTN |
| C7orf2 | FYN | DYM | ADRBK1 | PSMD8 | CA2 |
| NUCKS | ZMYM2 | SOX6 | SLC38A2 | RIN2 | CA9 |
| DKFZP566D1346 | CACNA1G | ARHGEF2 | IL8RA | RYBP | CDH3 |
| LOC388279 | SLC25A16 | ZCCHC6 | TAS2R14 | SDF4 | CDS1 |
| FLJ31795 | FLII | PPP3CA | CD300LB | SETD5 | COL17A1 |
| 6orf107 | EIF1 | FAM70B | GIPC3 | SPP1 | CORO1A |
| FLJ12439 | SEPT6 | TMED5 | MYCBP2 | LUZP1 | TCHP |
| FLJ12806 | PHF15 | FLJ43663 | FLJ90709 | FBLN1 | CDKN2C |
| FLJ39370 | NUP188 | HPS1 | PCTK2 | IGFBP3 | VCAN |
| GATS | ABR | MEF2A | PDE4DIP | DCN | CD44 |
| CCDC92 | CNR1 | ST3GAL5 | KIAA0194 | PRRX1 | STARD13 |
| FMNL2 | LOC283824 | SMYD3 | HOM-TES-103 | ANXA6 | SNED1 |
| ARID1B | FSTL4 | KLF7 | ENPP2 | PVRL3 | ZBTB38 |
| ZFHX1B | DNM1 | LOC200230 | CITED2 | MAP1B | SDC2 |
| SSBP2 | APOBEC3G | RERE | ZEB1 | TNFAIP6 | TPM1 |
| ARID5B | ATP2B1 | QKI | NID2 | CYBRD1 | COPZ2 |
| LOC157381 | SMPD1 | BICD1 | SEMA5A | FBN1 | STC1 |
| KPNA3 | SLC11A1 | CTNNB1 | DAB2 | NID1 | CDH1 |
| ARHGAP24 | FXYD5 | POU2F2 | KCNMA1 | OLFML3 | KRT5 |
| CCND2 | C14orf139 | EIF4ENIF1 | PTX3 | SNAI1 | KRT6B |
| VIM | SH3BGRL3 | BTG1 | PCDH9 | SNAI2 | EPCAM |
| CREB3L1 | TAGLN | CD24 | BGN | SYNC | GLYR1 |
| PALM2 | | | | | |

### Level of Expression of a Gene Isoform

In an embodiment, the invention features acquiring a value or values that is a function of the level of expression of a plurality of gene isoforms of a plurality of genes from a first and/or second and/or third and/or fourth and/or fifth and/or sixth and/or eighth and/or ninth and/or tenth and/or eleventh and/or twelfth and/or thirteenth set of genes. In an embodiment, a value for the level of expression of a gene isoform of a gene is acquired. In an embodiment, a value for the level of expression of a gene isoform of a gene; a plurality of gene isoforms of a gene; each gene isoform of a plurality of gene isoforms of a gene; a plurality of gene isoforms of a plurality of genes; and/or each gene isoform of a plurality of gene isoforms of a plurality of genes is acquired. In an embodiment, a value for the level of expression of a gene isoform of a gene; a plurality of gene isoforms of a gene; each gene isoform of a plurality of gene isoforms of a gene; a plurality of gene isoforms of a plurality of genes; and/or each gene isoform of a plurality of gene isoforms of a plurality of genes is assayed. In an embodiment, the level of expression of said gene isoform or plurality of gene isoforms is a function of the level of an alternatively spliced exon or plurality of alternatively spliced exons. In an embodiment, the level of said alternatively spliced exon or said plurality of alternatively spliced exons is acquired. In an embodiment, the level of said alternatively spliced exon or said plurality of alternatively spliced exons is assayed. In an embodiment, the level of expression of; said gene isoform or plurality of gene isoforms and/or said alternatively spliced exon or plurality of alternatively spliced exons is assayed in the whole subject sample. In an embodiment, the level of expression of; said gene isoform or plurality of gene isoforms and/or said alternatively spliced exon or plurality of alternatively spliced exons is assayed in a subregion of the subject sample, e.g., subregions of a tissue sample.

In an embodiment, the level of expression of; said gene isoform or plurality of gene isoforms and/or said alternatively spliced exon or plurality of alternatively spliced exons is assayed by detecting a protein product, e.g., an alternatively spliced protein. In an embodiment, the level of expression of; said gene isoform or plurality of gene isoforms and/or said alternatively spliced exon or plurality of alternatively spliced exons is assayed by detecting an alternatively spliced protein. In an embodiment, the level of expression; said gene isoform or plurality of gene isoforms and/or said alternatively spliced exon or plurality of alternatively spliced exons is assayed using antibodies specific for said alternatively spliced protein. In an embodiment, the level of expression of; said gene isoform or plurality of gene isoforms and/or said alternatively spliced exon or plurality of alternatively spliced exons is assayed using antibodies selective for said alternatively spliced exon.

In an embodiment, the level of expression of; said gene isoform or plurality of gene isoforms and/or said alternatively spliced exon or plurality of alternatively spliced exons is assayed by an immunohistochemistry technique. In an embodiment, the level of expression of; said gene isoform or plurality of gene isoforms and/or said alternatively spliced exon or plurality of alternatively spliced exons is assayed by an immunohistochemistry technique specific for said alternatively spliced protein. In an embodiment, the level of expression of; said gene isoform or plurality of gene isoforms and/or said alternatively spliced exon or plurality of alternatively spliced exons is assayed by an immunohistochemistry technique, using antibodies specific for said alternatively spliced protein. In an embodiment, the level of expression of; said gene isoform or plurality of gene isoforms and/or said alternatively spliced exon or plurality of alternatively spliced exons is assayed by an immunohistochemistry technique specific for said alternatively spliced exon. In an embodiment, the level of expression of; said gene isoform or plurality of gene isoforms and/or said alternatively spliced exon or plurality of alternatively spliced exons is assayed by an immunohistochemistry technique, using antibodies specific for said alternatively spliced exon.

In an embodiment, the level of expression of; said gene isoform or plurality of gene isoforms and/or said alternatively spliced exon or plurality of alternatively spliced exons is assayed by an immunoassay, e.g., Western blot, ELISA. In an embodiment, the level of expression of; said gene isoform or plurality of gene isoforms and/or said alternatively spliced exon or plurality of alternatively spliced exons is assayed by an immunoassay specific for said alternatively spliced protein. In an embodiment, the level of expression of; said gene isoform or plurality of gene isoforms and/or said alternatively spliced exon or plurality of alternatively spliced exons is assayed by an immunoassay, using antibodies specific for said alternatively spliced protein. In an embodiment, the level of expression of; said gene isoform or plurality of gene isoforms and/or said alternatively spliced exon or plurality of alternatively spliced exons is assayed by an immunoassay specific for said alternatively spliced exon. In an embodiment, the level of expression of; said gene isoform or plurality of gene isoforms and/or said alternatively spliced exon or plurality of alternatively spliced exons is assayed by an immunoassay, using antibodies specific for said alternatively spliced exon. In another embodiment, the level of expression of; said gene isoform or plurality of gene isoforms and/or said alternatively spliced exon or plurality of alternatively spliced exons is assayed using protein activity assays, such as functional assays.

In an embodiment, the level of expression of; said gene isoform or plurality of gene isoforms and/or said alternatively spliced exon or plurality of alternatively spliced exons is assayed by detecting an RNA product, e.g., mRNA of said sample. In an embodiment, the level of expression of; said gene isoform or plurality of gene isoforms and/or said alternatively spliced exon or plurality of alternatively spliced exons is assayed by a hybridization based method, e.g., hybridization with a probe that is specific for said alternatively spliced exon. In an embodiment, the level of expression of; said gene isoform or plurality of gene isoforms and/or said alternatively spliced exon or plurality of alternatively spliced exons is assayed by; applying said sample, or the mRNA isolated from, or amplified from, said sample, to a nucleic acid microarray, or chip array. In an embodiment, the level of expression of; said gene isoform or plurality of gene isoforms and/or said alternatively spliced exon or plurality of alternatively spliced exons is assayed by microarray, e.g., exon microarray.

In an embodiment, the level of expression of; said gene isoform or plurality of gene isoforms and/or said alternatively spliced exon or plurality of alternatively spliced exons, is assayed by a polymerase chain reaction (PCR) based method, e.g., quantitative reverse transcription coupled to polymerase chain reaction (qRT-PCR). In an embodiment, the level of expression of; said gene isoform or plurality of gene isoforms and/or said alternatively spliced exon or plurality of alternatively spliced exons, is assayed by a sequencing based method. In an embodiment, the level of expression of; said gene isoform or plurality of gene isoforms and/or said alternatively spliced exon or plurality of alternatively spliced exons, is assayed by quantitative RNA sequencing. In an embodiment, the level of expression of; said gene isoform or plurality of gene isoforms and/or said alternatively spliced exon or plurality of alternatively spliced exons, is assayed by an RNA in situ hybridization technique. In an embodiment, the level of expression of; said gene isoform or plurality of gene isoforms and/or said alternatively spliced exon or plurality of alternatively spliced exons, is measured by exon specific probes. In an embodiment, the level of expression of a plurality of said alternatively spliced exons is measured by a plurality of exon specific probes.

In an embodiment, the level of expression of; said gene isoform or plurality of gene isoforms and/or said alternatively spliced exon or plurality of alternatively spliced exons, is assayed by one or more exon specific probesets in Table 1, Table 2, Table 3, Table 4, Table 5, Table 6, Table 8, Table 9, Table 10, Table 11, Table 12, and/or Table 13. In an embodiment, the level of expression of; said gene isoform or plurality of gene isoforms and/or said alternatively spliced exon or plurality of alternatively spliced exons, is assayed by one or more exon specific probesets in Table 1, Table 2, Table 3, Table 4, Table 5, and/or Table 6; and other probesets related to detecting specific splicing events. In an embodiment, the level of expression of; said gene isoform or plurality of gene isoforms and/or said alternatively spliced exon or plurality of alternatively spliced exons is assayed by a plurality of exon specific probes in Table 1, Table 2, Table 3, Table 4, Table 5, Table 6, Table 8, Table 9, Table 10, Table 11, Table 12, and/or Table 13.

### Level of RNA Expression

In an embodiment, the invention features the acquisition of a value for the level of gene expression of a gene. In an embodiment, the invention features the acquisition of a value for the level of gene expression of a plurality of genes. In an embodiment, the invention features the acquisition of a value for the level of gene expression of each gene of a plurality of genes. In an embodiment, said gene or plurality of genes is in Table 7. In an embodiment, the level of gene expression is a function of the level of RNA expression of said plurality of genes. In an embodiment, the level of gene expression is a function of the level of RNA expression of each gene of said plurality of genes. In an embodiment, the level of RNA expression is acquired. In an embodiment, the level of RNA expression of said plurality of genes is assayed. In an embodiment, the level of RNA expression is assayed by detecting an RNA product, e.g., mRNA of said sample. In an embodiment, the level of RNA expression is assayed by a hybridization based method, e.g., hybridization with a probe that is specific for said RNA product. In an embodiment, the level of RNA expression is assayed by; applying said sample, or the mRNA isolated from, or amplified from; said sample, to a nucleic acid microarray, or chip array. In an embodiment, the level of RNA expression is assayed by microarray. In an embodiment, the level of RNA expression is assayed by a polymerase chain reaction (PCR) based method, e.g., qRT-PCR. In an embodiment, the level of RNA expression is assayed by a sequencing based method. In an embodiment, the level of RNA expression is assayed by quantitative RNA sequencing. In an embodiment, the level of RNA expression is assayed by RNA in situ hybridization. In an embodiment, the level of RNA expression is assayed in the whole subject sample. In an embodiment, the level of RNA expression is assayed in a subregion of the subject sample, e.g., subregions of a tissue sample.

In an embodiment, the level of gene expression is a function of the level of protein expression of a plurality of genes in said gene isoform sets one and/or two and/or three and/or four and/or five and/or six and/or seven. In an embodiment, the level of gene expression is a function of the level of protein expression of said plurality of genes. In an embodiment, the level of gene expression is a function of the level of protein expression of each gene of said plurality of genes. In an embodiment, the level of protein expression is acquired. In an embodiment, the level of protein expression is assayed. In an embodiment, the level of protein expression is assayed by detecting a protein product. In an embodiment, the level of protein expression is assayed using antibodies selective for said protein product. In an embodiment, the level of protein expression is assayed by an immunohistochemistry technique. In an embodiment, the level of protein expression is assayed by an immunohistochemistry technique, using antibodies specific for said protein product. In an embodiment, the level of protein expression is assayed by an immunoassay, e.g., Western blot, enzyme linked immunosorbant assay (ELISA). In an embodiment, the level of protein expression is assayed by an immunoassay specific for said protein. In an embodiment, levels of gene expression are assessed using protein activity assays, such as functional assays. In an embodiment, the level of protein expression is assayed in the whole subject sample. In an embodiment, the level of protein expression is assayed in a subregion of the subject sample, e.g., subregions of a tissue sample.

### Subject Sample

In an embodiment, the method of the invention features acquiring a subject sample, e.g., blood, urine, or tissue sample. In an embodiment, the subject sample is a tissue sample, e.g., biopsy. In an embodiment, the subject sample is a bodily fluid, e.g., blood, plasma, urine, saliva, sweat, tears, semen, or cerebrospinal fluid. In an embodiment, the subject sample is a bodily product, e.g., exhaled breath. In an embodiment, said subject sample is a tissue sample, wherein said tissue sample is derived from fixed tissue, paraffin embedded tissue, fresh tissue, or frozen tissue. In an embodiment, said subject sample is a tissue sample, wherein said tissue sample is fixed tissue, paraffin embedded tissue, fresh tissue, or frozen tissue.

In an embodiment, said subject sample is derived from a tumor. In an embodiment, said subject sample is obtained from a tumor sample. In an embodiment, said subject sample is a tumor sample. In an embodiment, said subject sample is obtained from tumor tissue. In an embodiment, the subject sample is tumor tissue. In an embodiment, said subject sample is obtained from tumor tissue, wherein said subject sample is fixed tumor tissue, paraffin embedded tumor tissue, fresh tumor tissue, or frozen tumor tissue. In an embodiment, said subject sample is a tissue sample, wherein said tissue sample is fixed, paraffin embedded, fresh, or frozen. In an embodiment, said subject sample is fixed, paraffin embedded, fresh, frozen, or fixed paraffin embedded tumor tissue.

In an embodiment, the subject sample is derived from a biopsy. In an embodiment, said subject sample derived from said biopsy is fresh tissue. In an embodiment, said subject sample derived from said biopsy is tumor tissue. In an embodiment, said subject sample derived from said biopsy is non-tumor tissue. In an embodiment, said subject sample is derived from a fine needle aspirate biopsy; large core needle biopsy; or directional vacuum assisted biopsy. In an embodiment, the subject sample is a tissue sample, wherein said tissue sample is derived from a fine needle aspirate; large core needle biopsy; or directional vacuum assisted biopsy.

In an embodiment, the subject sample is blood. In an embodiment, the subject sample is blood in which circulating tumor cells have been captured or isolated. In an embodiment, the subject sample is said circulating tumor cells that have been captured or isolated from said blood.

### Location Specific Acquisition of the Level of Gene Expression

In an embodiment, the invention features, acquiring a value or values for locations in a subject sample. In an embodiment, a value or values is acquired for a plurality of locations in a subject sample. In an embodiment, a first value or values is acquired for a first location in said subject sample. In an embodiment, a second value or values is acquired for a second location in said subject sample. In an embodiment, said first value or values is different from said second value or values. In an embodiment, the invention features, determining if said first value or values and said second value or values has a preselected relationship with a reference criterion. In an embodiment, determination of whether said first value or values and/or said second value or values has a preselected relationship with a reference criterion includes comparing said first value or values with said second value or values.

In an embodiment, said first value or values is associated with an increased likelihood of comprising a cancer stem cell, cancer associated mesenchymal cell, or tumor initiating cancer cell; than is said second value or values. In an embodiment, said first value or values is associated with a higher likelihood of comprising a cancer stem cell than is said second value or values. In an embodiment, said first value or values is associated with a higher likelihood of comprising a cancer associated mesenchymal cell than is said second value or values. In an embodiment, said first value or values is associated with a higher likelihood of comprising a tumor initiating cancer cell than is said second value or values. In an embodiment, said first value or values is indicative of a cancer stem cell, cancer associated mesenchymal cell, or tumor initiating cancer cell. In an embodiment, said first value or values is indicative of a cancer stem cell. In an embodiment, said first value or values is indicative of a cancer associated mesenchymal cell. In an embodiment, said first value or values is indicative of a tumor initiating cancer cell.

In an embodiment, the invention features, classifying a location in a subject sample as a cancer stem cell, cancer associated mesenchymal cell, or tumor initiating cancer cell. In an embodiment, the invention features, classifying said location as a cancer stem cell or non-cancer stem cell. In an embodiment, the invention features, classifying said location as a cancer stem cell. In an embodiment, the invention features, classifying said location as a non-cancer stem cell. In an embodiment, the invention features, classifying said location as a cancer associated mesenchymal cell. In an embodiment, the invention features, classifying said location as a tumor initiating cancer cell. In an embodiment, the invention features, acquiring a first value or values for a first location in said subject sample, wherein responsive to said first value or values, classifying said first location as comprising a cancer stem cell or non-cancer stem cell. In an embodiment, the invention features, acquiring a first value or values for a first location in said subject sample, wherein responsive to said first value or values, classifying said first location as comprising a cancer stem cell, cancer associated mesenchymal cell, or tumor initiating cancer cell.

In an embodiment, the invention features, acquiring a first value or values for a first location in a subject sample, wherein responsive to said first value or values, classifying said first location as comprising a cancer stem cell. In an embodiment, the invention features, acquiring a first value or values for a first location in said subject sample, wherein responsive to said first value or values, classifying said first location as comprising a non-cancer stem cell. In an embodiment, the invention features, acquiring a first value or values for a first location in a subject sample, wherein responsive to said first value or values, classifying said first location as comprising a cancer associated mesenchymal cell. In an embodiment, the invention features, acquiring a first value or values for a first location in a subject sample, wherein responsive to said first value or values, classifying said first location as comprising a tumor initiating cancer cell.

In an embodiment, said first location is classified as a cancer stem cell, cancer associated mesenchymal cell, or tumor initiating cancer cell. In an embodiment, said first location is classified as a cancer stem cell. In an embodiment, said first location is classified as a cancer associated mesenchymal cell. In an embodiment, said first location is classified as a tumor initiating cancer cell. In an embodiment, said first location is classified as a non-cancer stem cell. In an embodiment, said first location comprises a cancer stem cell, cancer associated mesenchymal cell, or tumor initiating cancer cell. In an embodiment, said first location comprises a cancer stem cell. In an embodiment, said first location comprises a cancer associated mesenchymal cell. In an embodiment, said first location comprises a tumor initiating cancer cell. In an embodiment, said first location comprises a non-cancer stem cell. In an embodiment, said first location is indicative of a cancer stem cell, cancer associated mesenchymal cell, or tumor initiating cancer cell. In an embodiment, said first location is indicative of a cancer stem cell. In an embodiment, said first location is indicative of a cancer associated mesenchymal cell. In an embodiment, said first location is indicative of a tumor initiating cancer cell. In an embodiment, said first location is indicative of a non-cancer stem cell.

In an embodiment, said first location comprises a subject sample. In an embodiment, said first location comprises a whole subject sample. In an embodiment, said first location comprises a sub-region of the subject sample. In an embodiment, said first location and said second location are separated by zero microns, i.e., said first location and second location are adjoining. In an embodiment, said first location and said second location are separated by more than zero microns; by more than ten microns; by more than twenty microns; by more than thirty microns; by more than forty microns; by more than fifty microns; by more than sixty microns; by more than seventy microns; by more than eighty microns; by more than ninety microns; or by more than one hundred microns. In an embodiment, said first location and said second location are separated by more than one thousand microns. In an embodiment, said first location and said second location are separated by at least ten microns; in an embodiment, said first location and said second location are separated by at least twenty microns; by at least thirty microns; by at least forty microns; by at least fifty microns; by at least sixty microns; by at least seventy microns; by at least eighty microns; by at least ninety microns; or by at least one hundred microns. In an embodiment, said first location and said second location are separated by more than one hundred microns. In an embodiment, said first location and said second location are separated by more than two hundred microns; three hundred microns; four hundred microns; five hundred microns; six hundred microns; seven hundred microns; eight hundred microns; nine hundred microns; or one thousand microns. In an embodiment, said first location and said second location are separated by at least one thousand microns. In an embodiment, said first location and said second location are separated by the maximum distance two locations of said subject sample can be separated. In an embodiment, said first location and said second location are separated by a distance between and including, zero and the maximum distance two locations of said subject sample can be separated.

In an embodiment, the average distance between said first location and said second location is more than zero microns; in an embodiment, the average distance between said first location and said second location is approximately ten microns; approximately twenty microns; approximately thirty microns; approximately forty micron; approximately fifty microns; approximately sixty microns; approximately seventy microns; approximately eighty microns; approximately ninety microns; or approximately one hundred microns. In an embodiment, the average distance between said first location and said second location is more than approximately fifty microns.

In an embodiment, the average distance between said first location and said second location is zero microns; in an embodiment, the average distance between said first location and said second location is more than ten microns; more than twenty microns; more than thirty microns; more than forty micron; more than fifty microns; more than sixty microns; more than seventy microns; more than eighty microns; more than ninety microns; or more than one hundred microns.

In an embodiment, the average distance between said first location and said second location is more than approximately one hundred microns. In an embodiment, the average distance between said first location and said second location is more than approximately two hundred; more than approximately three hundred; more than approximately four hundred; more than approximately five hundred; more than approximately six hundred; more than approximately seven hundred; more than approximately eight hundred; more than approximately nine hundred; or more than approximately one thousand microns. In an embodiment, the average distance between said first location and said second location is more than one thousand microns.

In an embodiment, the average distance between said first location and said second location is at least approximately ten microns; at least approximately twenty microns; at least approximately thirty microns; at least approximately forty microns; at least approximately fifty microns; at least approximately sixty microns; at least approximately seventy microns; at least approximately eighty microns; at least approximately ninety microns; at least approximately one hundred microns; at least approximately two hundred microns.

In an embodiment, said first value or values of said first location is a function of the level of gene expression of a gene at said first location. In embodiment, said first value or values is a function of the level of gene expression of a plurality of genes at said first location. In an embodiment, said first value or values is a function of the level of gene expression of each gene isoform of a plurality of genes at said first location. In an embodiment, the invention features the first value or values of said first location is a function of the level of gene expression of a gene or a plurality of genes at said first location, and responsive to said first value or values classifying said first location as a cancer stem cell or non cancer stem cell. In an embodiment, the invention features the first value or values of said first location is a function of the level of gene expression of a gene or a plurality of genes at said first location, and responsive to said first value or values classifying said first location as a cancer stem cell, cancer associated mesenchymal cell, or tumor initiating cancer cell. In an embodiment, said gene or said plurality of genes is in Table 1. In an embodiment, the level of gene expression is a function of the level of RNA expression of said gene or said plurality of genes. In an embodiment, the level of RNA expression of said gene or plurality of genes is assayed. In an embodiment, the level of RNA expression is assayed by detecting an RNA product. In an embodiment, the level of RNA expression is assayed by RNA in situ hybridization. In an embodiment, the level of gene expression is a function of the level of protein expression of said gene or said plurality of genes. In an embodiment, the level of protein expression is acquired. In an embodiment, the level of protein expression is assayed. In an embodiment, the level of protein expression is assayed by detecting a protein product. In an embodiment, the level of protein expression is assayed using antibodies selective for said protein product. In an embodiment, the level of protein expression is assayed by immunohistochemistry.

In an embodiment, a first value or values of said first location is a function of the level of expression of a gene isoform of a gene at said first location. In an embodiment, said first value or values is a function of the level of expression of a plurality of gene isoforms of a gene at said first location. In an embodiment, said first value or values is a function of the level of gene expression of each of a plurality of gene isoforms of a gene at said first location. In an embodiment, said first value or values is a function of the level of gene expression of each of a plurality of gene isoforms of a plurality of genes at said first location. In an embodiment, said first value or values is a function of the level of gene expression of a plurality of gene isoforms of a plurality of genes at said first location. In an embodiment, said gene or said plurality of genes is in Table 2. In an embodiment, the invention features a first value or values of said first location is a function of the level of expression of a gene isoform or plurality of gene isoforms at said first location, and responsive to said first value or values classifying said first location as a cancer stem cell or non cancer stem cell. In an embodiment, the invention features a first value or values of said first location is a function of the level of expression of a gene isoform or a plurality of gene isoforms at said first location, and responsive to said first value or values classifying said first location as a cancer stem cell, cancer associated mesenchymal cell, or tumor initiating cancer cell.

In an embodiment, the level of expression of said gene isoform or plurality of gene isoforms is a function of the level of expression of an alternatively spliced exon or a plurality of alternatively spliced exons. In an embodiment, the level of expression of said gene isoform or said plurality of gene isoforms is assayed. In an embodiment, the level of expression of said alternatively spliced exon or said plurality of alternatively spliced exons is assayed. In an embodiment, the level of expression of; said gene isoform or plurality of gene isoforms and/or said alternatively spliced exon or plurality of alternatively spliced exons, is assayed by detecting an RNA product. In an embodiment, the level of expression of; said gene isoform or plurality of gene isoforms and/or said alternatively spliced exon or plurality of alternatively spliced exons, is assayed by RNA in situ hybridization. In an embodiment, the level of expression of; said gene isoform or plurality of gene isoforms and/or said alternatively spliced exon or plurality of alternatively spliced exons, is assayed by detecting a protein product of said gene. In an embodiment, the level of expression of; said gene isoform or said plurality of gene isoforms and/or said alternatively spliced exon or said plurality of alternatively spliced exons, is assayed by detecting an alternatively spliced protein. In an embodiment, the level of expression of; said gene isoform or said plurality of gene isoforms and/or said alternatively spliced exon or said plurality of alternatively spliced exons, is assayed using antibodies specific for said alternatively spliced protein. In an embodiment, the level of expression of; said gene isoform or said plurality of gene isoforms and/or said alternatively spliced exon or said plurality of alternatively spliced exons, is assayed using antibodies specific for said alternatively spliced exon. In an embodiment, the level of expression of; said gene isoform or said plurality of gene isoforms and/or said alternatively spliced exon or said plurality of alternatively spliced exons, is assayed by immunohistochemistry.

In an embodiment, the invention features, a first value or values of said first location that is a function of the level of gene expression of a gene or a plurality of genes at said first location, and the acquisition of a value or values that is the function of the level of expression of a gene isoform or plurality of gene isoforms at said first location. In an embodiment, the invention features, a first value or values of said first location that is a function of the level of gene expression of a gene or a plurality of genes at said first location, and the acquisition of a value or values that is a function of the level of expression of a gene isoform of a gene at said first location. In an embodiment, the invention features, a first value or values of said first location that is a function of the level of gene expression of a gene or a plurality of genes at said first location, and the acquisition of a value or values that is a function of the level of expression of a plurality of gene isoforms of a gene at said first location. In an embodiment, the invention features, a first value or values of said first location that is a function of the level of gene expression of a gene or a plurality of genes at said first location, and the acquisition of a value or values that is a function of the level of expression of each gene isoform of a plurality of gene isoforms of a gene at said first location. In an embodiment, the invention features, a first value or values of said first location that is a function of the level of gene expression of a gene or a plurality of genes at said first location, and the acquisition of a value or values that is a function of the level of expression of a plurality of gene isoforms of a plurality of genes at said first location. In an embodiment, the invention features, a first value or values of said first location that is a function of the level of gene expression of a gene or a plurality of genes at said first location, and the acquisition of a value or values that is a function of the level of expression of each gene isoform of a plurality of gene isoforms of a plurality of genes at said first location. In an embodiment, the level of expression of said gene isoform or said plurality of gene isoforms is a function of the level of expression of an alternatively spliced exon or said plurality of alternatively spliced exons. In an embodiment, said gene isoform or plurality of gene isoforms is of a gene or plurality of genes in Table 1, Table 2, Table 3, Table 4, Table 5, Table 6, Table 8, Table 9, Table 10, Table 11, Table 12, and/or Table 13.

In an embodiment, the invention features, a first value or values of said first location that is a function of the level of gene expression of a gene or a plurality of genes at said first location, and the acquisition of a value or values that is the function of the level of expression of a gene isoform or plurality of gene isoforms at said first location; wherein responsive to said value or values classifying said first location as a cancer stem cell or non-cancer stem cell. In an embodiment, the invention features, a first value or values of said first location that is a function of the level of gene expression of a gene or a plurality of genes at said first location, and the acquisition of a value or values that is the function of the level of expression of a gene isoform or plurality of gene isoforms at said first location; wherein responsive to said value or values classifying said first location as a cancer stem cell, cancer associated mesenchymal cell, or tumor initiating cancer cell.

In an embodiment, the invention features, a first value or values of said first location that is a function of the level of gene expression of a gene or a plurality of genes at said first location, and the level of expression of a gene isoform of a gene at said first location. In an embodiment, the invention features, a first value or values of said first location that is a function of the level of gene expression of a gene or a plurality of genes at said first location, and the level of expression of a plurality of gene isoforms of a gene at said first location. In an embodiment, the invention features, a first value or values of said first location that is a function of the level of gene expression of a gene or a plurality of genes at said first location, and the level of expression of each gene isoform of a plurality of gene isoforms of a gene at said first location. In an embodiment, the invention features, a first value or values of said first location that is a function of the level of gene expression of a gene or a plurality of genes at said first location, and the level of expression of a plurality of gene isoforms of a plurality of genes. In an embodiment, the invention features, a first value or values of said first location that is a function of the level of gene expression of a gene or a plurality of genes at said first location, and the level of expression of each gene isoform of a plurality of gene isoforms of a plurality of genes.

In an embodiment, the level of expression of said gene isoform or said plurality of gene isoforms is a function of the level of expression of an alternatively spliced exon or a plurality of alternatively spliced exons. In an embodiment, said gene isoform or plurality of gene isoforms is of a gene or plurality of genes in Table 1, Table 2, Table 3, Table 4, Table 5, Table 6, Table 8, Table 9, Table 10, Table 11, Table 12, and/or Table 13. In an embodiment, the invention features, a first value or values of said first location that is a function of the level of gene expression of a gene or a plurality of genes at said first location, and the level of expression of a gene isoform of a gene at said first location; wherein responsive to said first value or values classifying said first location as a cancer stem cell or non-cancer stem cell. In an embodiment, the invention features, a first value or values of said first location that is a function of the level of gene expression of a gene or a plurality of genes at said first location, and the level of expression of a gene isoform of a gene at said first location; responsive to said first value or values classifying said first location as a cancer stem cell, cancer associated mesenchymal cell, or tumor initiating cancer cell.

### Administration

In an embodiment, the invention features administering an agent that inhibits or kills cancer associated mesenchymal cells, tumor initiating cancer cells, or cancer stem cells. In an embodiment, said agent that inhibits or kills cancer associated mesenchymal cells, tumor initiating cancer cells or cancer stem cells is administered to said subject. In an embodiment, the agent that inhibits or kills cancer associated mesenchymal cells, tumor initiating cancer cells, or cancer stem cells is selected from, e.g., salinomycin; a gamma secretase inhibitor; a DLL4 inhibitor, e.g., a therapeutic antibody targeting DLL4; a TRAIL inhibitor, e.g., a therapeutic antibody targeting TRAIL; a Hedgehog inhibitor, e.g., a therapeutic antibody targeting Hedgehog; a NOTCH3 inhibitor, e.g., a therapeutic antibody targeting NOTCH3; a NOTCH4 inhibitor, e.g., a therapeutic antibody targeting NOTCH4; a panNOTCH inhibitor, e.g., a therapeutic antibody targeting panNOTCH; a FGFR1 inhibitor, e.g., a therapeutic antibody targeting FGR1; a FGFR2 inhibitor, e.g., a therapeutic antibody targeting FGR2; a FGFR3 inhibitor, e.g., a therapeutic antibody targeting FGR3; a FGFR4 inhibitor, e.g., a therapeutic antibody targeting FGR4; a RON inhibitor, e.g., a therapeutic antibody targeting RON; Wnt pathway inhibitor, e.g., therapeutic antibodies targeting the Wnt pathway; a PI3Kinase inhibitor; a mTOR inhibitor; sodium meta arsenite; verapail; reserpine; a perifosen inhibitor of FAK1; a FAK inhibitor; a p38 inhibitor.

In an embodiment, the method features selecting a regimen, e.g., dosage, formulation, route of administration, number of dosages, or adjunctive therapies, of the agent that inhibits or kills cancer associated mesenchymal cells, tumor initiating cancer cells, or cancer stem cells. In an embodiment, said selecting is responsive to said value or values that is a function of the level of expression of a plurality of gene isoforms selected from said first and/or second and/or third and/or fourth and/or fifth and/or sixth and/or seventh set of gene isoforms.

In an embodiment, the invention features administering an agent that inhibits or kills cancer associated mesenchymal cells, tumor initiating cancer cells, or cancer stem cells to the subject according to the selected regimen. In an embodiment, said administration is provided responsive to acquiring knowledge or information of said value or values from another party. In an embodiment, said administration is provided responsive to an identification of said value or values, wherein said identification arises from collaboration with another party. In an embodiment, the invention features receiving a communication of the presence of said value or values that is a function of the level of expression of a plurality of gene isoforms selected from said first and/or second and/or third and/or fourth and/or fifth and/or sixth and/or seventh set of gene isoforms in a subject. In an embodiment, the acquisition of said value or values is at the time of or after diagnosis of cancer in said subject. In an embodiment, the acquisition of said value or values is post diagnosis of said cancer in the subject. In an embodiment, said subject has cancer. In an embodiment, the cancer is characterized as comprising cancer associated mesenchymal cells, tumor initiating cancer cells, or cancer stem cells. In an embodiment, the cancer is characterized as comprising cancer associated mesenchymal cells. In an embodiment, the cancer is characterized as comprising tumor initiating cancer cells. In an embodiment, the cancer is characterized as comprising cancer stem cells. In an embodiment, the cancer is characterized as being enriched with cancer associated mesenchymal cells, tumor initiating cancer cells, or cancer stem cells. In an embodiment, the cancer is characterized as being enriched with cancer associated mesenchymal cells. In an embodiment, the cancer is characterized as being enriched with tumor initiating cancer cells. In an embodiment, the cancer is characterized as being enriched with cancer stem cells.

In an embodiment, said cancer is an epithelial cell cancer. In an embodiment, said cancer is breast, lung, pancreatic, colorectal, prostate, head and neck, melanoma, acute myelogenous leukemia, glioblastoma, triple negative breast cancer, basal-like breast cancer, or claudin-low breast cancer. In another embodiment, said cancer is breast cancer. In an embodiment, said cancer is triple negative breast cancer. In an embodiment, the cancer is basal-like breast cancer. In an embodiment, the cancer is claudin-low breast cancer. In an embodiment, said cancer is recurrent, i.e., cancer that returns following treatment, and after a period of time in which said cancer was undetectable. In another embodiment, said cancer is a primary tumor, i.e., located at the anatomical site of tumor growth initiation. In an embodiment, said cancer is metastatic, i.e., appearing at a second anatomical site other than the anatomical site of tumor growth initiation.

In an embodiment of the invention, the value or values that is a function of the level of expression of a plurality of gene isoforms selected from said first and/or second and/or third and/or fourth and/or fifth and/or sixth and/or seventh set of gene isoforms; is acquired prior to, during, or after administration of a treatment to said subject. In an embodiment, said value or values is acquired prior to the administration of a treatment to said subject. In an embodiment, said value or values is acquired during the administration of a treatment to said subject. In an embodiment, said value or values is acquired after the administration of a treatment to said subject. In an embodiment, said subject is a non-responder, to said treatment. In an embodiment, said treatment is an anti-cancer treatment, e.g., chemotherapeutic agent, radiation treatment, surgery, etc. In an embodiment, said anti-cancer treatment is a chemotherapeutic agent. In an embodiment, said chemotherapeutic agent may include but is not limited to is one or more of the following chemotherapeutic agents: alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, vinblastine, and paclitaxel (Taxol)); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide). In an embodiment, said chemotherapeutic agent is selected from one or more of the following chemotherapeutic agents: Capecitabine, Carboplatin, Cisplatin, Cyclophosphamide, Docetaxel, Doxorubicin, Epirubicin, Eribulin, mesylate5-Fluorouracil, Gemcitabine, Ixabepilone, Liposomal doxorubicin, Methotrexate, Paclitaxel, or Vinorelbine; or any combination thereof.

In an embodiment, the invention features administering an agent that inhibits or kills cancer associated mesenchymal cells, tumor initiating cancer cells, or cancer stem cells and a second treatment. In an embodiment, said second treatment is an anti-cancer agent. In an embodiment, said second treatment is an agent that inhibits or kills cancer associated mesenchymal cells, tumor initiating cancer cells, or cancer stem cells. In an embodiment, said second treatment is not an agent that inhibits or kills cancer associated mesenchymal cells, tumor initiating cancer cells, or cancer stem cells. In an embodiment, said second treatment kills or inhibits growth of non-cancer stem cells in the subject. In an embodiment, the second treatment kills or inhibits growth of cancer cells that are not cancer stem cells, cancer associated mesenchymal cells, or tumor initiating cancer cells. In an embodiment, the second treatment is an anti-cancer treatment that does not target cancer stem cells, cancer associated mesenchymal cells, or cancer stem cells. In an embodiment, the second treatment is an anti-cancer treatment that does not primarily target cancer stem cells, cancer associated mesenchymal cells, or cancer stem cells. In an embodiment, said second treatment kills or inhibits growth of non-cancer associated mesenchymal cells, non-tumor initiating cancer cells, or non-cancer stem cells in the subject. In an embodiment, said second treatment is a chemotherapeutic agent. In an embodiment, said second treatment may include but is not limited to one or more of the following: alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, vinblastine, and paclitaxel (Taxol)); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide). In an embodiment, said second therapeutic agent is selected from Capecitabine, Carboplatin, Cisplatin, Cyclophosphamide, Docetaxel, Doxorubicin, Epirubicin, Eribulin, mesylate5-Fluorouracil, Gemcitabine, Ixabepilone, Liposomal doxorubicin, Methotrexate, Paclitaxel, or Vinorelbine; or any combination thereof. In an embodiment, the invention features further administering an agent that inhibits or kills cancer associated mesenchymal cells, tumor initiating cells, or cancer stem cells and more than one additional therapeutic agent.

In an embodiment, the invention includes, responsive to the acquisition of said value or values that is a function of the level of expression of a plurality of gene isoforms selected from said first and/or second and/or third and/or fourth and/or fifth and/or sixth and/or seventh set of gene isoforms; further stratifying a patient population. In an embodiment, the invention features, responsive to the acquisition of said value or values; further identifying or selecting said subject as likely or unlikely to respond positively to a treatment. In another embodiment, the invention features, responsive to the acquisition of said value or values; further selecting a treatment. In another embodiment, the invention features, responsive to the acquisition of said value or values; further prognosticating the time course of the disease in the subject. In an embodiment, said disease is a cancer. In an embodiment, the invention features, responsive to the acquisition of said value or values, one or more of the following: stratifying a patient population, identifying or selecting said subject as likely or unlikely to respond to a treatment, selecting a treatment option, prognosticating the time course of the disease in the subject; measuring the response at the end of therapy and predicting the long term outcome; and/or determining the cancer stem cell population as a predictor of response to a treatment or therapy.

### Genotype

In an embodiment, the method of the invention features the acquisition of a genotype of said subject sample. The subject sample can be any suitable subject sample including those subject samples previously mentioned. In an embodiment, said subject sample is a tumor sample. In an embodiment, at least one nucleotide of the subject sample is sequenced to determine the presence or absence of at least one genetic event associated with cancer. In an embodiment, at least one oncogene or tumor suppressor gene in the sample is sequenced. In an embodiment, the oncogene or oncogenes or tumor suppressor gene or tumor suppressor genes may include but is not limited to one or any combination of: Abl, Af4/hrx, akt-2, alk, alk/npm, aml 1, aml 1/mtg8, APC, axl, bcl-2, bcl-3, bcl-6, bcr/abl, brca-1, brca-2, beta-catenin, CDKN2, c-myc, c-sis, dbl, dek/can, E2A/pbx1, egfr, en1/hrx, erg/TLS, erbB, erbB-2, erk, ets-1, ews/fli-1, fms, fos, fps, gli, gsp, HER2/neu, hox11, hst, IL-3, int-2, jun, kit, KS3, K-sam, Lbc, lck, lmo1, lmo2, L-myc, lil-1, lyt-10, lyt-10/C alpha1, mas, mdm-2, mll, mos, mtg8/aml1, myb, myc, MYH11/CBFB, neu, nm23, N-myc, ost, p53, pax-5, pbx1/E2A, pdgfr, PI3-K, pim-1, PRAD-1, raf, RAR/PML, rash, rasK, rasN, Rb, rel/nrg, ret, rhom1, rhom2, ros, ski, sis, set/can, src, tal1, tal2, tan-1, telomerase, Tiam1, TSC2, trk, vegfr, or wnt.

### Reports

In an embodiment, the present invention features optionally providing a prediction of the likelihood that a subject will respond positively or will not respond positively to treatment with an agent that inhibits or kills cancer associated mesenchymal cells, tumor initiating cancer cells, or cancer stem cells. In an embodiment, said prediction is in the form of a report. In an embodiment, said predication includes a recommendation of whether said subject should be treated with a preselected drug, or treatment with a preselected drug should be withheld. In an embodiment, said preselected drug is an anti-cancer agent. In an embodiment, said preselected drug is an agent that inhibits or kills cancer associated mesenchymal cells, tumor initiating cancer cells, or cancer stem cells. In an embodiment, said agent that inhibits or kills cancer associated mesenchymal cells, tumor initiating cancer cells, or cancer stem cells is selected from: e.g., salinomycin; a gamma secretase inhibitor; a DLL4 inhibitor, e.g., a therapeutic antibody targeting DLL4; a TRAIL inhibitor, e.g., a therapeutic antibody targeting TRAIL; a Hedgehog inhibitor, e.g., a therapeutic antibody targeting Hedgehog; a NOTCH3 inhibitor, e.g., a therapeutic antibody targeting NOTCH3; a NOTCH4 inhibitor, e.g., a therapeutic antibody targeting NOTCH4; a panNOTCH inhibitor, e.g., a therapeutic antibody targeting panNOTCH; a FGFR1 inhibitor, e.g., a therapeutic antibody targeting FGR1; a FGFR2 inhibitor, e.g., a therapeutic antibody targeting FGR2; a FGFR3 inhibitor, e.g., a therapeutic antibody targeting FGR3; a FGFR4 inhibitor, e.g., a therapeutic antibody targeting FGR4; a RON inhibitor, e.g., a therapeutic antibody targeting RON; Wnt pathway inhibitor, e.g., therapeutic antibodies targeting the Wnt pathway; a PI3Kinase inhibitor; a mTOR inhibitor; sodium meta arsenite; verapail; reserpine; a perifosen inhibitor of FAK1; a FAK inhibitor; a p38 inhibitor.

### Kits or Products

In an aspect, the present invention includes a kit or product comprising a first agent capable of interacting with a gene expression product of a gene from a first and/or second and/or third and/or fourth and/or fifth and/or sixth and/or eighth and/or ninth and/or tenth and/or eleventh and/or twelfth and/or thirteenth set of gene isoforms. In an embodiment, the first set of gene isoforms (gene isoform set 1) comprises or consists of the gene isoforms in Table 1, Table 2, Table 3, Table 4, Table 5, and Table 6; the second set of gene isoforms (gene isoform set 2) comprises or consist of the gene isoforms in Table 1; the third set of gene isoforms (gene isoform set 3) comprises or consists of the gene isoforms in Table 2; the fourth set of gene isoforms (gene isoform set 4) comprises or consists of the gene isoforms in Table 3; the fifth set of gene isoforms (gene isoform set 5) comprises or consists of the gene isoforms in Table 4; and the sixth set of gene isoforms (gene isoform set 6) comprises or consists of the gene isoforms in Table 5; and the seventh set of gene isoforms (gene isoform set 7) comprises or consists of the gene isoforms in Table 6; and the eighth set of gene isoforms (gene isoform set 8) comprises or consists of the gene isoforms in Table 8; and the ninth set of gene isoforms (gene isoform set 9) comprises or consists of the gene isoforms in Table 9; and the tenth set of gene isoforms (gene isoform set 10) comprises or consists of the gene isoforms in Table 10; and the eleventh set of gene isoforms (gene isoform set 11) comprises or consists of the gene isoforms in Table 11; and the twelfth set of gene isoforms (gene isoform set 12) comprises or consists of the gene isoforms in Table 12; and the thirteenth set of gene isoforms (gene isoform set 13) comprises or consists of the gene isoforms in Table 13.

In an embodiment, said kit or product features a second agent capable of interacting with a gene expression product from said first and/or second and/or third and/or fourth and/or fifth and/or sixth and/or seventh set of gene isoforms. In an embodiment, said kit or product features a plurality of agents capable of interacting with a gene expression product from said first and/or second and/or third and/or fourth and/or fifth and/or sixth and/or seventh set of gene isoforms. In an embodiment, said kit or product features a plurality of agents capable of interacting with a plurality of gene expression products from said first and/or second and/or third and/or fourth and/or fifth and/or sixth and/or seventh and/or said eighth and/or said ninth and/or said tenth and/or said eleventh and/or said twelfth and/or said thirteenth set of gene isoforms. In an embodiment, said agent is a plurality of antibodies. In an embodiment, said agent is a plurality of oligonucleotides. In an embodiment, said agent is a plurality of antibodies and oligonucleotides. In an embodiment, said gene expression product is a RNA product. In an embodiment, said gene expression product is a protein product.

In an embodiment, said kit or product features an agent capable of interacting with a gene expression product of a gene in Table 7. In an embodiment, said kit or product contains plurality of agents capable of interacting with a plurality of genes in Table 7. In an embodiment, said kit or product features an agent capable of interacting with a gene expression product of a gene not in said first and/or second and/or third and/or fourth and/or fifth and/or sixth and/or seventh set of gene isoforms. In an embodiment, said kit or product features a plurality of agents capable of interacting with a gene expression product of a plurality of genes not in said first and/or second and/or third and/or fourth and/or fifth and/or sixth and/or seventh set of gene isoforms.

A kit or product comprising a first agent capable of interacting with a gene expression product of a plurality of genes from a first and/or second and/or third and/or fourth and/or fifth and/or sixth and/or seventh and/or eighth and/or ninth and/or tenth and/or eleventh and/or twelfth set of gene isoforms, wherein:
(i) said first set of gene isoforms comprises or consists of genes in Table 1,
(ii) said second set of gene isoforms comprises or consists of gene isoforms in Table 2; and
(iii) said third set of gene isoforms comprises or consists of gene isoforms in Table 3; and
(iv) said fourth set of gene isoforms comprises or consists of gene isoforms in Table 4; and
(v) said fifth set of gene isoforms comprises or consists of gene isoforms in Table 5; and
(vi) said sixth set of gene isoforms comprises or consists of gene isoforms in Table 6; and
(vii) said seventh set of gene isoforms comprises or consists of genes in Table 8,
(viii) said eighth set of gene isoforms comprises or consists of gene isoforms in Table 9; and
(ix) said ninth set of gene isoforms comprises or consists of gene isoforms in Table 10; and
(xi) said tenth set of gene isoforms comprises or consists of gene isoforms in Table 11; and
(xii) said eleventh set of gene isoforms comprises or consists of gene isoforms in Table 12; and
(xiii) said twelfth set of gene isoforms comprises or consists of gene isoforms in Table 13.

In one embodiment, the kit or product comprises a second agent capable of interacting with a gene expression product of a plurality of genes from said first and/or second and/or third and/or fourth and/or fifth and/or sixth and/or seventh and/or eighth and/or ninth and/or tenth and/or eleventh and/or twelfth set of gene isoforms. In one embodiment, the kit or product comprises a plurality of agents capable of interacting with a gene expression product of a plurality of genes from said first and/or second and/or third and/or fourth and/or fifth and/or sixth and/or seventh and/or eighth and/or ninth and/or tenth and/or eleventh and/or twelfth set of gene isoforms. In one embodiment, the kit or product comprises a plurality of agents capable of interacting with a plurality of gene expression products of a plurality of genes from said first and/or second and/or third and/or fourth and/or fifth and/or sixth and/or seventh and/or eighth and/or ninth and/or tenth and/or eleventh and/or twelfth set of gene isoforms.

In one embodiment, said agent is a plurality of antibodies. In one embodiment, said agent is a plurality of oligonucleotides. In one embodiment, said gene expression product is a RNA product. In one embodiment, said gene expression product is a protein product. In one embodiment, a value for the level of gene expression product for each gene isoform is assayed. In one embodiment, a value for the level of gene expression product for each gene isoform is assayed by detecting a protein product. In one embodiment, the protein product is detected by an immunoassay, e.g., immunohistochemistry. In one embodiment, a value for the level of gene expression product for each gene isoform is assayed by detecting a RNA product. In one embodiment, the RNA product is detected by a hybridization based method. In one embodiment, the RNA product is detected by microarray. In one embodiment, said microarray is an exon microarray. In one embodiment, the RNA product is detected by a polymerase chain reaction based method. In one embodiment, the RNA product is detected by a sequencing based method. In one embodiment, the RNA product is detected by a quantitative RNA sequencing.

In one embodiment, the gene expression products are derived from a tumor sample, e.g., a preparation of a primary tumor, metastatic tumor, lymph node, circulating tumor cells, ascites, or pleural effusion, plasma, serum, circulating, and interstitial fluid levels.

In one embodiment, a value for the level of gene expression product for each gene is determined. In one embodiment, a value that is a function of the level of gene expression for each gene is determined. In one embodiment, the value is compared to a reference standard, e.g., the level of expression of a control gene in the tumor sample.

In one embodiment, the kit or product further comprises the performance of an algorithm on a computer system to determine a value or values that is a function of a location of a gene expression product in the subject sample and/or a function of a level of a gene expression product of a gene in the subject sample. In one embodiment, the algorithm compares a ratio of the level of gene expression product of at least one of the genes selected from the group: HAS2, BIN1, PCOLCE, FERMT2, CTGF, IGFBP3, NID2, SLC44A1, FKBP5, and MLPH; to the level of gene expression product of at least one of the genes selected from the group: CDH1, and Cytokeratin.

In one embodiment, the kit or product further comprises a plurality of agents capable of interacting with at least one gene expression product selected from the group: CTGF, IGFBP3, TNFAIP6, NID2, HAS2, CCL2, MLPH, NID1, IGFBP4, FBLN5, and PCOLCE. In one embodiment, the kit or product further comprises a plurality of agents capable of interacting with a gene expression product of each gene isoform from the set of gene isoforms consisting of: CTGF, IGFBP3, TNFAIP6, NID2, HAS2, CCL2, MLPH, NID1, IGFBP4, FBLN5, and PCOLCE.

A kit or product comprising a first agent capable of interacting with a gene expression product of a plurality of genes from a first and/or second and/or third and/or fourth and/or fifth and/or sixth set of gene isoforms, wherein:
(i) said first set of gene isoforms comprises or consists of genes in Table 8,
(ii) said second set of gene isoforms comprises or consists of gene isoforms in Table 9; and
(iii) said third set of gene isoforms comprises or consists of gene isoforms in Table 10; and
(iv) said fourth set of gene isoforms comprises or consists of gene isoforms in Table 11; and
(v) said fifth set of gene isoforms comprises or consists of gene isoforms in Table 12; and
(vi) said sixth set of gene isoforms comprises or consists of gene isoforms in Table 13.

In one embodiment, the kit or product comprises a second agent capable of interacting with a gene expression product of a plurality of genes from said first and/or second and/or third and/or fourth and/or fifth and/or sixth set of gene isoforms. In one embodiment, the kit or product comprises a plurality of agents capable of interacting with a gene expression product of a plurality of genes from said first and/or second and/or third and/or fourth and/or fifth and/or sixth set of gene isoforms. In one embodiment, the kit or product comprises a plurality of agents capable of interacting with a plurality of gene expression products of a plurality of genes from said first and/or second and/or third and/or fourth and/or fifth and/or sixth set of gene isoforms.

In one embodiment, said agent is a plurality of antibodies. In one embodiment, said agent is a plurality of oligonucleotides. In one embodiment, said gene expression product is a RNA product. In one embodiment, said gene expression product is a protein product. In one embodiment, a value for the level of gene expression product for each gene isoform is assayed. In one embodiment, a value for the level of gene expression product for each gene isoform is assayed by detecting a protein product. In one embodiment, the protein product is detected by an immunoassay, e.g., immunohistochemistry. In one embodiment, a value for the level of gene expression product for each gene isoform is assayed by detecting a RNA product. In one embodiment, the RNA product is detected by a hybridization based method. In one embodiment, the RNA product is detected by microarray. In one embodiment, said microarray is an exon microarray. In one embodiment, the RNA product is detected by a polymerase chain reaction based method. In one embodiment, the RNA product is detected by a sequencing based method. In one embodiment, the RNA product is detected by a quantitative RNA sequencing.

In one embodiment, the gene expression products are derived from a tumor sample, e.g., a preparation of a primary tumor, metastatic tumor, lymph node, circulating tumor cells, ascites, or pleural effusion, plasma, serum, circulating, and interstitial fluid levels.

In one embodiment, a value for the level of gene expression product for each gene is determined. In one embodiment, a value that is a function of the level of gene expression for each gene is determined. In one embodiment, the value is compared to a reference standard, e.g., the level of expression of a control gene in the tumor sample.

In one embodiment, the kit or product further comprises the performance of an algorithm on a computer system to determine a value or values that is a function of a location of a gene expression product in the subject sample and/or a function of a level of a gene expression product of a gene in the subject sample. In one embodiment, the algorithm compares a ratio of the level of gene expression product of at least one of the genes selected from the group: HAS2, BIN1, PCOLCE, FERMT2, CTGF, IGFBP3, NID2, SLC44A1, FKBP5, and MLPH; to the level of gene expression product of at least one of the genes selected from the group: CDH1, and Cytokeratin.

In one embodiment, the kit or product further comprises a plurality of agents capable of interacting with at least one gene expression product selected from the group: CTGF, IGFBP3, TNFAIP6, NID2, HAS2, CCL2, MLPH, NID1, IGFBP4, FBLN5, and PCOLCE. In one embodiment, the kit or product further comprises a plurality of agents capable of interacting with a gene expression product of each gene isoform from the set of gene isoforms consisting of: CTGF, IGFBP3, TNFAIP6, NID2, HAS2, CCL2, MLPH, NID1, IGFBP4, FBLN5, and PCOLCE.

Methods of Assaying

In one aspect, methods described herein include methods of assaying in a subject sample the level of gene expression product of a plurality of gene isoforms from a first and/or second and/or third and/or fourth and/or fifth and/or sixth set of gene isoforms, wherein:

a gene from a first and/or second and/or third and/or fourth and/or fifth and/or sixth and/or seventh and/or eighth and/or ninth and/or tenth and/or eleventh and/or twelfth set of gene isoforms, wherein:
(i) said first set of gene isoforms comprises or consists of genes in Table 1,
(ii) said second set of gene isoforms comprises or consists of gene isoforms in Table 2; and
(iii) said third set of gene isoforms comprises or consists of gene isoforms in Table 3; and
(iv) said fourth set of gene isoforms comprises or consists of gene isoforms in Table 4; and
(v) said fifth set of gene isoforms comprises or consists of gene isoforms in Table 5; and
(vi) said sixth set of gene isoforms comprises or consists of gene isoforms in Table 6; and
(vii) said seventh set of gene isoforms comprises or consists of genes in Table 8,
(viii) said eighth set of gene isoforms comprises or consists of gene isoforms in Table 9; and
(ix) said ninth set of gene isoforms comprises or consists of gene isoforms in Table 10; and
(xi) said tenth set of gene isoforms comprises or consists of gene isoforms in Table 11; and
(xii) said eleventh set of gene isoforms comprises or consists of gene isoforms in Table 12; and
(xiii) said twelfth set of gene isoforms comprises or consists of gene isoforms in Table 13; comprising a first agent capable of interacting with a gene expression product of a plurality of genes selected from a first and/or second and/or third and/or fourth and/or fifth and/or sixth set of genes; and wherein the method comprises assaying the level of gene expression product of the plurality of genes.

In one embodiment, the method comprises a second agent capable of interacting with a gene expression product from said first and/or second and/or third and/or fourth and/or fifth and/or sixth and/or seventh and/or eighth and/or ninth and/or tenth and/or eleventh and/or twelfth set of gene isoforms. In one embodiment, the method comprises a plurality of agents capable of interacting with a gene expression product from said first and/or second and/or third and/or fourth and/or fifth and/or sixth and/or seventh and/or eighth and/or ninth and/or tenth and/or eleventh and/or twelfth set of gene isoforms. In one embodiment, the method comprises a plurality of agents capable of interacting with a plurality of gene expression products from said first and/or second and/or third and/or fourth and/or fifth and/or sixth and/or seventh and/or eighth and/or ninth and/or tenth and/or eleventh and/or twelfth set of gene isoforms.

In one embodiment, said agent is a plurality of antibodies. In one embodiment, said agent is a plurality of oligonucleotides. In one embodiment, said gene expression product is a RNA product. In one embodiment, said gene expression product is a protein product. In one embodiment, a value for the level of gene expression product for each gene isoform is assayed. In one embodiment, a value for the level of gene expression product for each gene isoform is assayed by detecting a protein product. In one embodiment, the protein product is detected by an immunoassay, e.g., immunohistochemistry. In one embodiment, a value for the level of gene expression product for each gene isoform is assayed by detecting a RNA product. In one embodiment, the RNA product is detected by a hybridization based method. In one embodiment, the RNA product is detected by microarray. In one embodiment, said microarray is an exon microarray. In one embodiment, the RNA product is detected by a polymerase chain reaction based method. In one embodiment, the RNA product is detected by a sequencing based method. In one embodiment, the RNA product is detected by a quantitative RNA sequencing.

In one embodiment, the gene expression products are derived from a tumor sample, e.g., a preparation of a primary tumor, metastatic tumor, lymph node, circulating tumor cells, ascites, or pleural effusion, plasma, serum, circulating, and interstitial fluid levels.

In one embodiment, a value for the level of gene expression product for each gene is determined. In one embodiment, a value that is a function of the level of gene expression for each gene is determined. In one embodiment, the value is compared to a reference standard, e.g., the level of expression of a control gene in the tumor sample.

In one embodiment, the method further comprises the performance of an algorithm on a computer system to determine a value or values that is a function of a location of a gene expression product in the subject sample and/or a function of a level of a gene expression product of a gene in the subject sample. In one embodiment, the algorithm compares a ratio of the level of gene expression product of at least one of the genes selected from the group: HAS2, BIN1, PCOLCE, FERMT2, CTGF, IGFBP3, NID2, SLC44A1, FKBP5, and MLPH; to the level of gene expression product of at least one of the genes selected from the group: CDH1, and Cytokeratin.

In one embodiment, the method further comprises a plurality of agents capable of interacting with at least one gene expression product selected from the group: CTGF, IGFBP3, TNFAIP6, NID2, HAS2, CCL2, MLPH, NID1, IGFBP4, FBLN5, and PCOLCE. In one embodiment, the method further comprises a plurality of agents capable of interacting with a gene expression product of each gene isoform from the set of gene isoforms consisting of: CTGF, IGFBP3, TNFAIP6, NID2, HAS2, CCL2, MLPH, NID1, IGFBP4, FBLN5, and PCOLCE.

Reaction Mixtures

In one aspect, reaction mixtures described herein include a reaction mixture comprising: a plurality of detection reagents; and a plurality of target nucleic acid molecules derived from a subject, wherein each of the plurality of detection reagents comprises a plurality probes to measure the level of gene expression product of a gene from a gene from a first and/or second and/or third and/or fourth and/or fifth and/or sixth and/or seventh and/or eighth and/or ninth and/or tenth and/or eleventh and/or twelfth set of gene isoforms, wherein:
(i) said first set of gene isoforms comprises or consists of genes in Table 1,
(ii) said second set of gene isoforms comprises or consists of gene isoforms in Table 2; and
(iii) said third set of gene isoforms comprises or consists of gene isoforms in Table 3; and
(iv) said fourth set of gene isoforms comprises or consists of gene isoforms in Table 4; and
(v) said fifth set of gene isoforms comprises or consists of gene isoforms in Table 5; and
(vi) said sixth set of gene isoforms comprises or consists of gene isoforms in Table 6; and
(vii) said seventh set of gene isoforms comprises or consists of genes in Table 8,
(viii) said eighth set of gene isoforms comprises or consists of gene isoforms in Table 9; and
(ix) said ninth set of gene isoforms comprises or consists of gene isoforms in Table 10; and
(xi) said tenth set of gene isoforms comprises or consists of gene isoforms in Table 11; and
(xii) said eleventh set of gene isoforms comprises or consists of gene isoforms in Table 12; and
(xiii) said twelfth set of gene isoforms comprises or consists of gene isoforms in Table 13.

In one embodiment, each probe comprises a DNA, RNA or mixed DNA/RNA molecule, which is complementary to a nucleic acid sequence on each of the plurality of target nucleic acid molecules, wherein each target nucleic acid molecule is derived from a gene in said a first and/or second and/or third and/or fourth and/or fifth and/or sixth and/or seventh and/or eighth and/or ninth and/or tenth and/or eleventh and/or twelfth set of gene isoforms. In one embodiment, each of the plurality of detection reagents comprises a probe to measure the expression at least 1, 2, 3, 4, 5, 6, 7, 8, 9, 10, 11, 12, 13, 14, 15, 16, 17, 18, 19, or 20 genes in said first and/or second and/or third and/or fourth and/or fifth and/or sixth set of gene isoforms. In one embodiment, each of the plurality of detection reagents comprises a probe to measure the expression 1, 2, 3, 4, 5, 6, 7, 8, 9, 10, 11, 12, 13, 14, 15, 16, 17, 18, 19, or 20 genes in said a first and/or second and/or third and/or fourth and/or fifth and/or sixth and/or seventh and/or eighth and/or ninth and/or tenth and/or eleventh and/or twelfth set of gene isoforms. In one embodiment, each of the plurality of detection reagents comprises a probe to measure the expression of no more than 1, 2, 3, 4, 5, 6, 7, 8, 9, 10, 11, 12, 13, 14, 15, 16, 17, 18, 19, or 20 genes in said a first and/or second and/or third and/or fourth and/or fifth and/or sixth and/or seventh and/or eighth and/or ninth and/or tenth and/or eleventh and/or twelfth set of gene isoforms. In one embodiment, each of the plurality of detection reagents comprises a probe to measure the expression of only genes in said a first and/or second and/or third and/or fourth and/or fifth and/or sixth and/or seventh and/or eighth and/or ninth and/or tenth and/or eleventh and/or twelfth set of gene isoforms.

In an embodiment, the probe is a nucleic acid molecule. In one embodiment, the plurality of target nucleic acid molecules is derived from a subject with cancer. Also described herein are kits comprising detection reagents described herein.

In one aspect, reaction mixtures described herein include a reaction mixture comprising:

a plurality of detection reagents, e.g., a plurality of substrates, e.g., a plurality of antibodies; and a plurality of target proteins derived from a cancer, wherein each of the plurality of target proteins is encoded by a gene in said a first and/or second and/or third and/or fourth and/or fifth and/or sixth and/or seventh and/or eighth and/or ninth and/or tenth and/or eleventh and/or twelfth set of gene isoforms, and wherein each of the plurality of detection reagents is a probe specific for one of the plurality of target proteins, e.g., binds to the target protein.

In one embodiment, each of the plurality of detection reagents comprises a probe to measure the expression at least 1, 2, 3, 4, 5, 6, 7, 8, 9, 10, 11, 12, 13, 14, 15, 16, 17, 18, 19, or 20 genes in said a first and/or second and/or third and/or fourth and/or fifth and/or sixth and/or seventh and/or eighth and/or ninth and/or tenth and/or eleventh and/or twelfth set of gene isoforms. In one embodiment, each of the plurality of detection reagents comprises a probe to measure the expression 1, 2, 3, 4, 5, 6, 7, 8, 9, 10, 11, 12, 13, 14, 15, 16, 17, 18, 19, or 20 genes in said a first and/or second and/or third and/or fourth and/or fifth and/or sixth and/or seventh and/or eighth and/or ninth and/or tenth and/or eleventh and/or twelfth set of gene isoforms. In one embodiment, each of the plurality of detection reagents comprises a probe to measure the expression of no more than 1, 2, 3, 4, 5, 6, 7, 8, 9, 10, 11, 12, 13, 14, 15, 16, 17, 18, 19, or 20 genes in said a first and/or second and/or third and/or fourth and/or fifth and/or sixth and/or seventh and/or eighth and/or ninth and/or tenth and/or eleventh and/or twelfth set of gene isoforms. In one embodiment, each of the plurality of detection reagents comprises a probe to measure the expression of only genes in said first and/or second and/or third and/or fourth and/or fifth and/or sixth and/or seventh and/or eighth and/or ninth and/or tenth and/or eleventh and/or twelfth set of gene isoforms.

In one embodiment, the plurality of target proteins is derived from a patient with a cancer. Also described herein are kits comprising detection reagents described herein.

Also described herein are methods of making a reaction mixture.

In one aspect, described herein are methods of making a reaction mixture comprising:

combining a plurality of detection reagents, with a plurality of target nucleic acid molecules derived from a patient with an ovarian cancer, wherein each target nucleic acid molecule is derived from a plurality of genes a first and/or second and/or third and/or fourth and/or fifth and/or sixth and/or seventh and/or eighth and/or ninth and/or tenth and/or eleventh and/or twelfth set of gene isoforms, wherein:
(i) said first set of gene isoforms comprises or consists of genes in Table 1,
(ii) said second set of gene isoforms comprises or consists of gene isoforms in Table 2; and
(iii) said third set of gene isoforms comprises or consists of gene isoforms in Table 3; and
(iv) said fourth set of gene isoforms comprises or consists of gene isoforms in Table 4; and
(v) said fifth set of gene isoforms comprises or consists of gene isoforms in Table 5; and
(vi) said sixth set of gene isoforms comprises or consists of gene isoforms in Table 6; and
(vii) said seventh set of gene isoforms comprises or consists of genes in Table 8,
(viii) said eighth set of gene isoforms comprises or consists of gene isoforms in Table 9; and
(ix) said ninth set of gene isoforms comprises or consists of gene isoforms in Table 10; and
(xi) said tenth set of gene isoforms comprises or consists of gene isoforms in Table 11; and
(xii) said eleventh set of gene isoforms comprises or consists of gene isoforms in Table 12; and
(xiii) said twelfth set of gene isoforms comprises or consists of gene isoforms in Table 13; and wherein each of the plurality of detection reagents comprises a probe to measure the expression of a gene in said first and/or second and/or third and/or fourth and/or fifth and/or sixth set of gene isoforms.

In one aspect, described herein are methods of making a reaction mixture comprising:

combining a plurality of detection reagents, e.g., a plurality of substrates, e.g., a plurality of antibodies; and a plurality of target proteins derived from an ovarian cancer, wherein each of the plurality of target proteins is encoded by a gene in said first and/or second and/or third and/or fourth and/or fifth and/or sixth and/or seventh and/or eighth and/or ninth and/or tenth and/or eleventh and/or twelfth set of gene isoforms, wherein:
(i) said first set of gene isoforms comprises or consists of genes in Table 1,
(ii) said second set of gene isoforms comprises or consists of gene isoforms in Table 2; and
(iii) said third set of gene isoforms comprises or consists of gene isoforms in Table 3; and
(iv) said fourth set of gene isoforms comprises or consists of gene isoforms in Table 4; and
(v) said fifth set of gene isoforms comprises or consists of gene isoforms in Table 5; and
(vi) said sixth set of gene isoforms comprises or consists of gene isoforms in Table 6; and
(vii) said seventh set of gene isoforms comprises or consists of genes in Table 8,
(viii) said eighth set of gene isoforms comprises or consists of gene isoforms in Table 9; and
(ix) said ninth set of gene isoforms comprises or consists of gene isoforms in Table 10; and
(xi) said tenth set of gene isoforms comprises or consists of gene isoforms in Table 11; and
(xii) said eleventh set of gene isoforms comprises or consists of gene isoforms in Table 12; and
(xiii) said twelfth set of gene isoforms comprises or consists of gene isoforms in Table 13; and wherein each of the plurality of detection reagents is a probe specific for one of the plurality of target proteins, e.g., binds to the target protein.

In one aspect, reaction mixtures described herein include a reaction mixture comprising: a plurality of detection reagents; and a plurality of target nucleic acid molecules derived from a subject, wherein each of the plurality of detection reagents comprises a plurality probes to measure the level of gene expression product of a gene from a first and/or second and/or third and/or fourth and/or fifth and/or sixth set of gene isoforms, wherein:
(i) said first set of gene isoforms comprises or consists of genes in Table 8,
(ii) said second set of gene isoforms comprises or consists of gene isoforms in Table 9; and
(iii) said third set of gene isoforms comprises or consists of gene isoforms in Table 10; and
(iv) said fourth set of gene isoforms comprises or consists of gene isoforms in Table 11; and
(v) said fifth set of gene isoforms comprises or consists of gene isoforms in Table 12; and
(vi) said sixth set of gene isoforms comprises or consists of gene isoforms in Table 13.

In one embodiment, each probe comprises a DNA, RNA or mixed DNA/RNA molecule, which is complementary to a nucleic acid sequence on each of the plurality of target nucleic acid molecules, wherein each target nucleic acid molecule is derived from a gene in said first and/or second and/or third and/or fourth and/or fifth and/or sixth set of gene isoforms. In one embodiment, each of the plurality of detection reagents comprises a probe to measure the expression at least 1, 2, 3, 4, 5, 6, 7, 8, 9, 10, 11, 12, 13, 14, 15, 16, 17, 18, 19, or 20 genes in said first and/or second and/or third and/or fourth and/or fifth and/or sixth set of gene isoforms. In one embodiment, each of the plurality of detection reagents comprises a probe to measure the expression 1, 2, 3, 4, 5, 6, 7, 8, 9, 10, 11, 12, 13, 14, 15, 16, 17, 18, 19, or 20 genes in said first and/or second and/or third and/or fourth and/or fifth and/or sixth set of gene isoforms. In one embodiment, each of the plurality of detection reagents comprises a probe to measure the expression of no more than 1, 2, 3, 4, 5, 6, 7, 8, 9, 10, 11, 12, 13, 14, 15, 16, 17, 18, 19, or 20 genes in said first and/or second and/or third and/or fourth and/or fifth and/or sixth set of gene isoforms. In one embodiment, each of the plurality of detection reagents comprises a probe to measure the expression of only genes in said first and/or second and/or third and/or fourth and/or fifth and/or sixth set of gene isoforms.

In an embodiment, the probe is a nucleic acid molecule. In one embodiment, the plurality of target nucleic acid molecules is derived from a subject with cancer. Also described herein are kits comprising detection reagents described herein.

In one aspect, reaction mixtures described herein include a reaction mixture comprising:

a plurality of detection reagents, e.g., a plurality of substrates, e.g., a plurality of antibodies; and a plurality of target proteins derived from a cancer, wherein each of the plurality of target proteins is encoded by a gene in said first and/or second and/or third and/or fourth and/or fifth and/or sixth set of gene isoforms, and wherein each of the plurality of detection reagents is a probe specific for one of the plurality of target proteins, e.g., binds to the target protein.

In one embodiment, each of the plurality of detection reagents comprises a probe to measure the expression at least 1, 2, 3, 4, 5, 6, 7, 8, 9, 10, 11, 12, 13, 14, 15, 16, 17, 18, 19, or 20 genes in said first and/or second and/or third and/or fourth and/or fifth and/or sixth set of gene isoforms. In one embodiment, each of the plurality of detection reagents comprises a probe to measure the expression 1, 2, 3, 4, 5, 6, 7, 8, 9, 10, 11, 12, 13, 14, 15, 16, 17, 18, 19, or 20 genes in said first and/or second and/or third and/or fourth and/or fifth and/or sixth set of gene isoforms. In one embodiment, each of the plurality of detection reagents comprises a probe to measure the expression of no more than 1, 2, 3, 4, 5, 6, 7, 8, 9, 10, 11, 12, 13, 14, 15, 16, 17, 18, 19, or 20 genes in said first and/or second and/or third and/or fourth and/or fifth and/or sixth set of gene isoforms. In one embodiment, each of the plurality of detection reagents comprises a probe to measure the expression of only genes in said first and/or second and/or third and/or fourth and/or fifth and/or sixth set of gene isoforms.

In one embodiment, the plurality of target proteins is derived from a patient with a cancer. Also described herein are kits comprising detection reagents described herein.

Also described herein are methods of making a reaction mixture.

In one aspect, described herein are methods of making a reaction mixture comprising:

combining a plurality of detection reagents, with a plurality of target nucleic acid molecules derived from a patient with an ovarian cancer, wherein each target nucleic acid molecule is derived from a plurality of genes a first and/or second and/or third and/or fourth and/or fifth and/or sixth set of gene isoforms, wherein:
(i) said first set of gene isoforms comprises or consists of genes in Table 8,
(ii) said second set of gene isoforms comprises or consists of gene isoforms in Table 9; and
(iii) said third set of gene isoforms comprises or consists of gene isoforms in Table 10; and
(iv) said fourth set of gene isoforms comprises or consists of gene isoforms in Table 11; and
(v) said fifth set of gene isoforms comprises or consists of gene isoforms in Table 12; and
(vi) said sixth set of gene isoforms comprises or consists of gene isoforms in Table 13, and wherein each of the plurality of detection reagents comprises a probe to measure the expression of a gene in said first and/or second and/or third and/or fourth and/or fifth and/or sixth set of gene isoforms.

In one aspect, described herein are methods of making a reaction mixture comprising:

combining a plurality of detection reagents, e.g., a plurality of substrates, e.g., a plurality of antibodies; and a plurality of target proteins derived from an ovarian cancer, wherein each of the plurality of target proteins is encoded by a gene in said first and/or second and/or third and/or fourth and/or fifth and/or sixth set of gene isoforms, a first and/or second and/or third and/or fourth and/or fifth and/or sixth set of gene isoforms, wherein:
(i) said first set of gene isoforms comprises or consists of genes in Table 8,
(ii) said second set of gene isoforms comprises or consists of gene isoforms in Table 9; and
(iii) said third set of gene isoforms comprises or consists of gene isoforms in Table 10; and
(iv) said fourth set of gene isoforms comprises or consists of gene isoforms in Table 11; and
(v) said fifth set of gene isoforms comprises or consists of gene isoforms in Table 12; and
(vi) said sixth set of gene isoforms comprises or consists of gene isoforms in Table 13, and wherein each of the plurality of detection reagents is a probe specific for one of the plurality of target proteins, e.g., binds to the target protein.

In Vitro Assays

Also described herein are in vitro methods and assays. In one aspect described herein are in vitro methods and assays of determining if a subject is a potential candidate for treatment with an agent that inhibits or kills cancer associated mesenchymal cells, tumor initiating cancer cells, or cancer stem cells, the method comprising determining the level of gene expression product of a plurality of genes selected from a first and/or second and/or third and/or fourth and/or fifth and/or sixth and/or seventh and/or eighth and/or ninth and/or tenth and/or eleventh and/or twelfth set of gene isoforms, wherein:
(i) said first set of gene isoforms comprises or consists of genes in Table 1,
(ii) said second set of gene isoforms comprises or consists of gene isoforms in Table 2; and
(iii) said third set of gene isoforms comprises or consists of gene isoforms in Table 3; and
(iv) said fourth set of gene isoforms comprises or consists of gene isoforms in Table 4; and
(v) said fifth set of gene isoforms comprises or consists of gene isoforms in Table 5; and
(vi) said sixth set of gene isoforms comprises or consists of gene isoforms in Table 6; and
(vii) said seventh set of gene isoforms comprises or consists of genes in Table 8,
(viii) said eighth set of gene isoforms comprises or consists of gene isoforms in Table 9; and
(ix) said ninth set of gene isoforms comprises or consists of gene isoforms in Table 10; and
(xi) said tenth set of gene isoforms comprises or consists of gene isoforms in Table 11; and
(xii) said eleventh set of gene isoforms comprises or consists of gene isoforms in Table 12; and
(xiii) said twelfth set of gene isoforms comprises or consists of gene isoforms in Table 13; and
optionally, administering the agent to the subject.

In some embodiments, the determining the level of gene expression product comprises determining the level of RNA expression of each gene isoform of said plurality of genes. In an embodiment, the level of gene expression is a function of the level of RNA expression of each gene isoform of said plurality of genes. In an embodiment, the level of RNA expression is acquired. In an embodiment, the level of RNA expression of said plurality of genes is assayed. In an embodiment, the level of RNA expression is assayed by detecting an RNA product, e.g., mRNA of said sample. In an embodiment, the level of RNA expression is assayed by a hybridization based method, e.g., hybridization with a probe that is specific for said RNA product. In an embodiment, the level of RNA expression is assayed by; applying said sample, or the mRNA isolated from, or amplified from; said sample, to a nucleic acid microarray, or chip array. In an embodiment, the level of RNA expression is assayed by microarray. In an embodiment, the level of RNA expression is assayed by a polymerase chain reaction (PCR) based method, e.g., qRT-PCR. In an embodiment, the level of RNA expression is assayed by a sequencing based method. In an embodiment, the level of RNA expression is assayed by quantitative RNA sequencing. In an embodiment, the level of RNA expression is assayed by RNA in situ hybridization. In an embodiment, the level of RNA expression is assayed in the whole subject sample. In an embodiment, the level of RNA expression is assayed in a subregion of the subject sample, e.g., subregions of a tissue sample.

In some embodiments, the determining the level of gene expression product comprises determining the level of protein expression of each gene isoform of said plurality of genes. In an embodiment, the level of protein expression is acquired. In an embodiment, the level of protein expression is assayed. In an embodiment, the level of protein expression is assayed by detecting a protein product. In an embodiment, the level of protein expression is assayed using antibodies selective for said protein product. In an embodiment, the level of protein expression is assayed by an immunohistochemistry technique. In an embodiment, the level of protein expression is assayed by an immunohistochemistry technique, using antibodies specific for said protein product. In an embodiment, the level of protein expression is assayed by an immunoassay, e.g., Western blot, enzyme linked immunosorbant assay (ELISA). In an embodiment, the level of protein expression is assayed by an immunoassay specific for said protein. In an embodiment, levels of gene expression are assessed using protein activity assays, such as functional assays. In an embodiment, the level of protein expression is assayed in the whole subject sample. In an embodiment, the level of protein expression is assayed in a subregion of the subject sample, e.g., subregions of a tissue sample.

In some embodiments, the method further comprises determining the level of gene expression product in a cell. In some embodiments, the determining the level of gene expression product in a cell comprises: contacting the cell with an agent; determining the level of gene expression product; and comparing the level of gene expression product to an appropriate control.

In some embodiments, the subject sample is a sample described herein, e.g., blood, urine, or tissue sample. In an embodiment, the subject sample is a tissue sample, e.g., biopsy. In an embodiment, the subject sample is a bodily fluid, e.g., blood, plasma, urine, saliva, sweat, tears, semen, or cerebrospinal fluid. In an embodiment, the subject sample is a bodily product, e.g., exhaled breath. In an embodiment, said subject sample is a tissue sample, wherein said tissue sample is derived from fixed tissue, paraffin embedded tissue, fresh tissue, or frozen tissue. In an embodiment, said subject sample is a tissue sample, wherein said tissue sample is fixed tissue, paraffin embedded tissue, fresh tissue, or frozen tissue.

In some embodiments the subject has cancer, e.g., a cancer described herein, e.g., breast cancer. The cancer can include cancers characterized as comprising cancer stem cells, cancer associated mesenchymal cells, or tumor initiating cancer cells. The cancer can include cancers that have been characterized as being enriched with cancer stem cells, cancer associated mesenchymal cells, or tumor initiating cancer cells. Exemplary cancers include epithelial cancers, breast, lung, pancreatic, colorectal, prostate, head and neck, melanoma, acute myelogenous leukemia, and glioblastoma. Exemplary breast cancers include triple negative breast cancer, basal-like breast cancer, claudin-low breast cancer, invasive, inflammatory, metaplastic, and advanced Her-2 positive or ER-positive cancers resistant to therapy. Other cancers include but are not limited to, brain, abdominal, esophagus, gastrointestinal, glioma, liver, tongue, neuroblastoma, osteosarcoma, ovarian, retinoblastoma, Wilm's tumor, multiple myeloma, skin, lymphoma, blood, retinal, acute lymphoblastic leukemia, bladder, cervical, kidney, endometrial, meningioma, lymphoma, skin, uterine, lung, non small cell lung, nasopharyngeal carcinoma, neuroblastoma, solid tumor, hematologic malignancy, leukemia, squamous cell carcinoma, testicular, thyroid, mesothelioma, brain vulval, sarcoma, intestine, oral, T cell leukemia, endocrine, salivary, spermatocytic seminoma, sporadic medulalry thyroid carcinoma, non-proliferating testes cells, cancers related to malignant mast cells, non-Hodgkin's lymphoma, and diffuse large B cell lymphoma.

The cancer can be a primary tumor, i.e., located at the anatomical site of tumor growth initiation. The cancer can also be metastatic, i.e., appearing at least a second anatomical site other than the anatomical site of tumor growth initiation. The cancer can be a recurrent cancer, i.e., cancer that returns following treatment, and after a period of time in which the cancer was undetectable. The recurrent cancer can be anatomically located locally to the original tumor, e.g., anatomically near the original tumor; regionally to the original tumor, e.g., in a lymph node located near the original tumor; or distantly to the original tumor, e.g., anatomically in a region remote from the original tumor.

Also described herein are in vitro methods and assays. In one aspect described herein are in vitro methods and assays of determining if a subject is a potential candidate for treatment with an agent that inhibits or kills cancer associated mesenchymal cells, tumor initiating cancer cells, or cancer stem cells, the method comprising determining the level of gene expression product of a plurality of genes selected from a first and/or second and/or third and/or fourth and/or fifth and/or sixth set of gene isoforms, in a subject sample, wherein:
(i) said first set of gene isoforms comprises or consists of genes in Table 8,
(ii) said second set of gene isoforms comprises or consists of gene isoforms in Table 9; and
(iii) said third set of gene isoforms comprises or consists of gene isoforms in Table 10; and
(iv) said fourth set of gene isoforms comprises or consists of gene isoforms in Table 11; and
(v) said fifth set of gene isoforms comprises or consists of gene isoforms in Table 12; and
(vi) said sixth set of gene isoforms comprises or consists of gene isoforms in Table 13; and
optionally, administering the agent to the subject.

In some embodiments, the determining the level of gene expression product comprises determining the level of RNA expression of each gene isoform of said plurality of genes. In an embodiment, the level of gene expression is a function of the level of RNA expression of each gene isoform of said plurality of genes. In an embodiment, the level of RNA expression is acquired. In an embodiment, the level of RNA expression of said plurality of genes is assayed. In an embodiment, the level of RNA expression is assayed by detecting an RNA product, e.g., mRNA of said sample. In an embodiment, the level of RNA expression is assayed by a hybridization based method, e.g., hybridization with a probe that is specific for said RNA product. In an embodiment, the level of RNA expression is assayed by; applying said sample, or the mRNA isolated from, or amplified from; said sample, to a nucleic acid microarray, or chip array. In an embodiment, the level of RNA expression is assayed by microarray. In an embodiment, the level of RNA expression is assayed by a polymerase chain reaction (PCR) based method, e.g., qRT-PCR. In an embodiment, the level of RNA expression is assayed by a sequencing based method. In an embodiment, the level of RNA expression is assayed by quantitative RNA sequencing. In an embodiment, the level of RNA expression is assayed by RNA in situ hybridization. In an embodiment, the level of RNA expression is assayed in the whole subject sample. In an embodiment, the level of RNA expression is assayed in a subregion of the subject sample, e.g., subregions of a tissue sample.

In some embodiments, the determining the level of gene expression product comprises determining the level of protein expression of each gene isoform of said plurality of genes. In an embodiment, the level of protein expression is acquired. In an embodiment, the level of protein expression is assayed. In an embodiment, the level of protein expression is assayed by detecting a protein product. In an embodiment, the level of protein expression is assayed using antibodies selective for said protein product. In an embodiment, the level of protein expression is assayed by an immunohistochemistry technique. In an embodiment, the level of protein expression is assayed by an immunohistochemistry technique, using antibodies specific for said protein product. In an embodiment, the level of protein expression is assayed by an immunoassay, e.g., Western blot, enzyme linked immunosorbant assay (ELISA). In an embodiment, the level of protein expression is assayed by an immunoassay specific for said protein. In an embodiment, levels of gene expression are assessed using protein activity assays, such as functional assays. In an embodiment, the level of protein expression is assayed in the whole subject sample. In an embodiment, the level of protein expression is assayed in a subregion of the subject sample, e.g., subregions of a tissue sample.

In some embodiments, the method further comprises determining the level of gene expression product in a cell. In some embodiments, the determining the level of gene expression product in a cell comprises: contacting the cell with an agent; determining the level of gene expression product; and comparing the level of gene expression product to an appropriate control.

In some embodiments, the subject sample is a sample described herein, e.g., blood, urine, or tissue sample. In an embodiment, the subject sample is a tissue sample, e.g., biopsy. In an embodiment, the subject sample is a bodily fluid, e.g., blood, plasma, urine, saliva, sweat, tears, semen, or cerebrospinal fluid. In an embodiment, the subject sample is a bodily product, e.g., exhaled breath. In an embodiment, said subject sample is a tissue sample, wherein said tissue sample is derived from fixed tissue, paraffin embedded tissue, fresh tissue, or frozen tissue. In an embodiment, said subject sample is a tissue sample, wherein said tissue sample is fixed tissue, paraffin embedded tissue, fresh tissue, or frozen tissue.

In some embodiments the subject has cancer, e.g., a cancer described herein, e.g., breast cancer. The cancer can include cancers characterized as comprising cancer stem cells, cancer associated mesenchymal cells, or tumor initiating cancer cells. The cancer can include cancers that have been characterized as being enriched with cancer stem cells, cancer associated mesenchymal cells, or tumor initiating cancer cells. Exemplary cancers include epithelial cancers, breast, lung, pancreatic, colorectal, prostate, head and neck, melanoma, acute myelogenous leukemia, and glioblastoma. Exemplary breast cancers include triple negative breast cancer, basal-like breast cancer, claudin-low breast cancer, invasive, inflammatory, metaplastic, and advanced Her-2 positive or ER-positive cancers resistant to therapy. Other cancers include but are not limited to, brain, abdominal, esophagus, gastrointestinal, glioma, liver, tongue, neuroblastoma, osteosarcoma, ovarian, retinoblastoma, Wilm's tumor, multiple myeloma, skin, lymphoma, blood, retinal, acute lymphoblastic leukemia, bladder, cervical, kidney, endometrial, meningioma, lymphoma, skin, uterine, lung, non small cell lung, nasopharyngeal carcinoma, neuroblastoma, solid tumor, hematologic malignancy, leukemia, squamous cell carcinoma, testicular, thyroid, mesothelioma, brain vulval, sarcoma, intestine, oral, T cell leukemia, endocrine, salivary, spermatocytic seminoma, sporadic medulalry thyroid carcinoma, non-proliferating testes cells, cancers related to malignant mast cells, non-Hodgkin's lymphoma, and diffuse large B cell lymphoma.

The cancer can be a primary tumor, i.e., located at the anatomical site of tumor growth initiation. The cancer can also be metastatic, i.e., appearing at least a second anatomical site other than the anatomical site of tumor growth initiation. The cancer can be a recurrent cancer, i.e., cancer that returns following treatment, and after a period of time in which the cancer was undetectable. The recurrent cancer can be anatomically located locally to the original tumor, e.g., anatomically near the original tumor; regionally to the original tumor, e.g., in a lymph node located near the original tumor; or distantly to the original tumor, e.g., anatomically in a region remote from the original tumor.

### BRIEF DESCRIPTION OF THE DRAWINGS

FIG. 1 illustrates exon normalization. The figure shows the raw probeset expression values for an example probeset group of an example gene. The figure compares the combined gene and exon expression level (top panel), the gene expression level (middle panel), and the gene expression normalized zero mean exon expression level (lower panel). The figure demonstrates the differential expression of particular exons of the example gene.

FIG. 2 is a flow chart which illustrates the skipped exon selection method. The figure outlines the method of skipped exon selection from algorithms that evaluate probeset values indicative of exons and genes. As shown in the flow chart, exon-level gene expression data originates from platforms such as Affymetrics exon array, RNA-sequencing strategies, and the like. A classification scheme is created to distinguish two groups, with example groups shown, such as Hi/Low EMT, Hi/Low Tumor-Initiating, Basal vs Luminal, and other signatures or classifiers. The flow chart shows that classifier data are processed using algorithms that examine exons and splicing events such as FIRMA, Splicing Index, MiDAS, etc. Statistical values are used to filter and rank the outputs using multiple statistical criteria, such as probeset p-value, multiple testing-adjusted algorithm p-values, etc. Highly ranked candidates are formed from the exon lists and concordant, class-specific, and union exon list groups are created.

FIG. 3 illustrates the skipped exon selection method, illustrating different exons in one gene. The skipped exon selection method is illustrated for probesets for the single gene ENAH, (hMENA). The top panel diagram illustrates the relative expression level of different exon probe sets of ENAH based on the colorization index on the right. In this example, the normalized relative expression level of all ENAH probesets (listed on left, ENAH exons/probesets with numeric values representing genomic position) was determined to vary between 3.08 and −4.33. The bottom panel diagram illustrates an EMT (epithelial-mesenchymal transition) gene set score ranking strategy applied to the exon probesets of ENAH. EMT gene set score refers to the gene set score formed for 41 human breast cancer cell lines, as labeled in the x-axis. EMT gene set scores range from 5 to −5 in this example. The dotted line delineates an arbitrary distinguisher between cell lines leftward that are more epithelial-like, and rightward cell lines that are more mesenchymal-like. INV, the ENAH INV exon 11a, is an ENAH exon that distributes to relatively high expression values in epithelial and a relatively low expression values in mesenchymal breast cancer cell lines.

FIG. 4 illustrates an epithelial-mesenchymal transition (EMT) discriminator for exon discovery. The figure illustrates the groups of exon probesets having differential expression between two classification types based on an EMT discriminator. Individual probesets are indicated by column entries. Individual human breast cancer cell lines are indicated by rows, and the cell lines fall into two basic types in this example, E (epithelial) or M (mesenchymal). The diagram indicates the probesets that are represented by M-deleted, E-included group, or by the M-included, E-deleted group. White indicates relatively high levels and black indicates relatively low levels for each exon probeset.

FIG. 5 illustrates a tumor initiating (TI, High) discriminator for exon discovery. The figure illustrates the groups of exon probesets having differential expression between two classification types based on a tumor initiating (TI) discriminator. Individual probesets are indicated by column entries. Individual human breast cancer cell lines are indicated by rows, and the cell lines fall into two basic types in this example, Hi or Low, based on a classifier. The diagram indicates the probesets that are represented by TI(High)-deleted, TI(Low)-included group, or by the TI(High)-included, TI(Low)-deleted group. White indicates relatively high levels and black indicates relatively low levels for each exon probeset.

FIG. 6 is a Venn diagram which illustrates M-included (EMT) included exon concordance amongst three breast cancer discriminators. The Venn diagram indicates the concordance of exon lists created from outputs of three FIRMA algorithms developed from exon array data of a group of human breast cancer cell lines. The subset that are M-included (EMT), high TI, or basal B-like are shown. The three FIRMA outputs were derived from EMT, TI, and basal-B vs luminal discriminators with the number of exon probesets shown in brackets. In this example, 40 exon probesets are concordant between the three groups.

FIG. 7 illustrates a concordant group amongst three breast cancer discriminators The figure illustrates the pattern of expression of the exon probesets from the three FIRMA algorithm outputs from evaluation of a large group of human breast cancer cell lines. Rows are exon probesets. Columns are human breast cancer cell lines. Unsupervised hierarchical clustering orders the cell lines by pattern similarity and the exon probesets by pattern similarity as illustrated.

FIG. 8 illustrates breast cancer cell lines with combined EMT and fibroblast-low discriminators for exon discovery. The figure illustrates the derivation of exon probesets having the features of high levels of differential expression between human breast cancer cell lines based on a discriminator classifier. The graph shows the group of exon probesets (rows) and their pattern of expression in the cell lines (columns) based on high expression to low expression. As the diagram indicates the exon probesets and the cell lines are ordered for similarity based on unsupervised hierarchical clustering. The top part of the figure diagrams the exon probeset clusters that are M-deleted, E-included, and Fibroblast-included. The bottom part of the figure diagrams the exon probeset clusters are those that are M-included, E-deleted, and fibroblast-deleted.

FIG. 9 illustrates the pattern of expression of four differentially expressed exons amongst human breast cancer cell lines. The figure illustrates the level of differential expression (y axis: exon differential) relative to the tumor initiating (TI) gene score amongst the group of human breast cancer cell lines in the evaluation. The values for several fibroblast cell lines are also plotted. The four exon probesets are NNT:2808443, B4GALNT1:3458723, RUNX1:3930506, and SEPT9:3735857 from four different genes that are differentially expressed.

FIG. 10 illustrates the pattern of expression of four differentially expressed exons amongst human triple negative breast cancer versus non-triple negative breast cancers The figure illustrates the level of differential expression (y axis: exon differential) relative to the tumor initiating (TI) gene score amongst the group of human breast cancer cell lines demonstrated to be of the triple negative breast cancer subtype, or demonstrated to be another subtype. The values for several fibroblast cell lines are also plotted. The four exon probesets are NNT:2808443, B4GALNT1:3458723, RUNX1:3930506, and SEPT9:3735857 from four different genes that are differentially expressed.

FIG. 11 illustrates the pattern of expression of four differentially expressed exons amongst human breast cancer cell lines. The figure illustrates the level of differential expression (y axis: exon differential) relative to the epithelial mesenchymal transition (EMT) gene set score amongst a group of human breast cancer cell lines in the evaluation. The values for several fibroblast cell lines are also plotted. The four exon probesets are NNT:2808443, B4GALNT1:3458723, RUNX1:3930506, and SEPT9:3735857 from four different genes that are differentially expressed.

FIG. 12 illustrates the determination of differentially expressed exon probesets derived from an alternative discriminator methodology as a union group for exon discovery. The figure illustrates the groups of exon probesets having differential expression between two classification types based on a confluence of three discriminators, tumor initiating (TI), EMT, and basal-B, that is applied using support vector machine processes and the splicing index exon algorithm. Individual probesets are indicated by row entries. Individual human breast cancer cell lines are indicated by columns. The cell lines fall into two basic types in this example, Hi or Low, based on a TI classifier. As shown, the hierarchical clustering falls into two primary groups. The figure indicates the probesets that are represented by M-included [TI(High)-included] group, or by the E-included [TI(Low)-included] group. Green indicates relatively low levels and red indicates relatively high levels for each exon probeset.

FIG. 13. Illustrates the determination of differentially expressed exon probesets derived from an alternative discriminator methodology as a concordant group for exon discovery. The figure illustrates the groups of exon probesets having differential expression between two classification types based on a confluence of three discriminators, tumor initiating (TI), EMT, and basal-B, that is applied using support vector machine processes and the splicing index exon algorithm. Individual probesets are indicated by row entries. Individual human breast cancer cell lines are indicated by columns, and the cell lines fall into two basic types in this example, Hi or Low, based on a TI classifier. As shown, the hierarchical clustering falls into two primary groups. The figure indicates the probesets that are represented by M-included [TI(High)-included] group, or by the E-included [TI(Low)-included] group. Green indicates relatively low levels and red indicates relatively high levels for each exon probeset. The individual 68 probesets are listed in the Tables 5 and 6 for this group that is the concordance of the 3 discriminator methods.

FIG. 14 is a Venn diagram which illustrates the concordance between the three discriminators for human breast cancer exon discovery. The Venn diagram indicates the concordant 68 exon probesets derived from the confluence of the three splicing index and support vector machine discriminators for TI, EMT, and basal-B versus luminal types.

FIG. 15 illustrates the pathway analysis for exon biomarker discovery. The figure indicates the output of high statistical significance from the KEGG and GO pathway analysis for the 209 exon probeset genes (˜150 genes). The −log 10 P values are ranging from 1 to 8 for the pathways shown.

FIG. 16 illustrates the hierarchical clustering of human tumor cell lines representing many different tumor types. The figure illustrates a hierarchical clustering analysis executed with the 209 exon probesets (union) where the samples are divisible into high tumor initiating and low tumor initiating subclasses.

FIG. 17 illustrates how the centroid model defines human breast cancer subgroups. The figure illustrates the output of a centroid model (two group classifier) for tumor initiating genes [TI gene centroid]. The upper panel illustrates the unsupervised hierarchical clustering of human breast cancers relative to the application of the TI gene centroid. The middle panel illustrates human primary breast cancers are also grouped by the TI gene centroid into TI (red) or non-TI (green), and black is an intermediate value. The lower panel illustrates human primary breast cancers are also grouped by gene expression values for the ER, PR, and HER2 genes and expression values are low (green), mid (black) or high (red). The black vertical lines are aligned with the major hierarchical clustering subgroups of the human primary breast cancers.

FIG. 18 illustrates how the concordant cancer stem cell (CSC) exon centroid model defines the human breast cancer tumor initiating subgroups. The figure illustrates the output of a CSC exon centroid model (two group classifier) for tumor initiating exons [TI 68 exon centroid]. The 68 exon probesets used in the exon signature for the centroid model are formed from the concordant group. The upper panel illustrates the unsupervised hierarchical clustering of human breast cancers relative to the application of the CSC exon centroid. The middle panel illustrates human primary breast cancers are also group by the CSC exon centroid into TI (red) or non-TI (green), and black is an intermediate value. The lower panel illustrates human primary breast cancers are also grouped by gene expression values for the ER, PR, and HER2 genes and expression values are low (green), mid (black) or high (red). The black vertical lines are aligned with the major hierarchical clustering subgroups of the human primary breast cancers.

FIG. 19 illustrates how the cancer stem cell (CSC) union 209 exon centroid model defines the human breast cancer tumor initiating subgroups. The figure illustrates the output of an exon centroid model (two group classifier) for CSC tumor initiating exons [CSC 209 exon centroid]. The 209 exon probesets used in the exon signature for the centroid model are formed from the concordant group. The upper panel illustrates the unsupervised hierarchical clustering of human breast cancers relative to the application of the CSC 209 exon centroid. The middle panel illustrates human primary breast cancers are also group by the CSC exon centroid into TI (red) or non-TI (green), and black is an intermediate value. The lower panel illustrates human primary breast cancers are also grouped by gene expression values for the ER, PR, and HER2 genes and expression values are low (green), mid (black) or high (red). The black vertical lines are aligned with the major hierarchical clustering subgroups of the human primary breast cancers.

FIG. 20 illustrates the cancer stem cell (CSC) centroid comparison between gene-based and exon-based centroids in human breast cancers. The figure illustrates the correlation between two centroids of different types as specified. CSC 209 SI exon centroid is on the y-axis. Gene centroid, TI gene signature is on the x-axis. Each dot represents a human breast cancer specimen where the application of the exon and gene centroids are evaluated for degree of similarity with 4 values for every human breast cancer specimen. Kappa value indicates overall similarity between the two groups. The illustrated exon-based and gene-based centroids have an overall kappa value of 0.60 that are highly significant.

FIG. 21 illustrates that the cancer stem cell (CSC) 68 exon centroid and tumor initiating gene centroid are highly correlated with triple negative breast cancer based on a gene signature. The figure illustrates the high degree of similarity between centroids and gene signatures for triple negative breast cancer. The left panel illustrates 68 exon centroid values and triple negative gene signature values for a group of primary human breast cancers. Pos_Triples (TNBC gene signature output per specimen), Slexon_posTI (TI 68 exon centroid, output per specimen). The right panel illustrates gene centroid values and triple negative gene signature values for a group of primary human breast cancers. Pos_Triples (TNBC gene signature output per specimen), geneTI (TI gene centroid, output per specimen). R(squared), R², are indicative of the high degree of similarities of the two groups (exon centroid: TNBC gene signature, R²=0.7337, and TI gene signature: TNBC gene signature, R²=0.6063, respectively).

FIG. 22 illustrates that the cancer stem cell (CSC) 209 exon centroid and tumor initiating gene centroid are highly correlated with triple negative breast cancer based on a gene signature. The figure illustrates the high degree of similarity between centroids and gene signatures for triple negative breast cancer. Exon centroid values and triple negative gene signature values for a group of primary human breast cancers. Pos_Triples (TNBC Gene Signature output per specimen), Slexon_posTI (TI 209 exon centroid, output per specimen). R(squared), R², are indicative of the high degree of similarities of the two groups (CSC 209 exon centroid: TNBC Gene signature, R²=0.8025).

### DETAILED DESCRIPTION

Certain terms are first defined. Additional terms are defined throughout the specification.

“Acquire” or “acquiring” as the terms are used herein, refer to obtaining possession of a physical entity, or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

“Acquiring a sample” as the term is used herein, refers to obtaining possession of a sample, e.g., a tissue sample or nucleic acid sample, by “directly acquiring” or “indirectly acquiring” the sample. “Directly acquiring a sample” means performing a process (e.g., performing a physical method such as a surgery or extraction) to obtain the sample. “Indirectly acquiring a sample” refers to receiving the sample from another party or source (e.g., a third party laboratory that directly acquired the sample). Directly acquiring a sample includes performing a process that includes a physical change in a physical substance, e.g., a starting material, such as a tissue, e.g., a tissue in a human patient or a tissue that has was previously isolated from a patient. Exemplary changes include making a physical entity from a starting material, dissecting or scraping a tissue; separating or purifying a substance (e.g., a sample tissue or a nucleic acid sample); combining two or more separate entities into a mixture; performing a chemical reaction that includes breaking or forming a covalent or non-covalent bond. Directly acquiring a sample includes performing a process that includes a physical change in a sample or another substance, e.g., as described above. As used herein, a subject who is a “candidate” is a one likely to respond to a particular therapeutic regimen, relative to a reference subject or group of subjects. A “non-candidate” subject is one not likely to respond to a particular therapeutic regimen, relative to a reference subject or group of subjects.

The term “cancer stem cell” refers to a cell or group of cells in a tumor having stem-like progenitor properties.

The term “tumor initiating cancer cell” refers to a cell with stem-like properties and the ability to initiate a tumor upon introduction into a tissue.

The term “cancer associated mesenchymal cell” refers to a cell or cells in a tumor that have acquired or retained mesenchymal properties.

The term “anti-cancer stem cell agent” refers to an inhibitor or killer of cancer stem cells causing a reduction or elimination of these cells or a reduction in the ability of these cells to proliferative or to survive the treatment.

The term “agent that inhibits or kills cancer associated mesenchymal cells” refers to an inhibitor or killer of cancer mesenchymal cells causing a reduction or elimination of these cells or a reduction in the ability of these cells to proliferative or to survive the treatment.

The term “agent that inhibits or kills tumor initiating cancer cells” refers to an inhibitor or killer of cells with stem-like properties and the ability to initiate a tumor upon introduction into a tissue.

The term “agent that kills or inhibits cancer stem cells” refers to an inhibitor or killer of cells or a group of cells in a tumor having stem-like progenitor properties.

The term “anti-cancer agent” refers to an inhibitor of cancer initiation, growth, progression, or metastasis

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion.

“Chemotherapeutic agent” means a chemical substance, such as a cytotoxic or cytostatic agent, that is used to treat a condition, particularly cancer. As used herein, “chemotherapy” and “chemotherapeutic” and “chemotherapeutic agent” are synonymous terms.

A “gene isoform” as used herein, refers to different size and compositions of mRNAs of the same gene. A list of alternatively spliced exon types that are included in the invention, are skipped exons, included introns, 5′ non-coding inclusions, 3 non-coding inclusions, and gene isoforms composed of combinations of these features. “Likely to” or “increased likelihood,” as used herein, refers to an increased probability that an item, object, thing or person will occur. Thus, in one example, a subject that is likely to respond to treatment with, alone or in combination, has an increased probability of responding to treatment with said agent that inhibits or kills cancer associated mesenchymal cells, tumor initiating cancer cells, or cancer stem cells alone or in combination, relative to a reference subject or group of subjects.

“Likely to” or “increased likelihood,” as used herein, refers to an increased probability that an item, object, thing or person will occur. Thus, in one example, a subject that is likely to respond to treatment with an agent that inhibits or kills cancer associated mesenchymal cells, tumor initiating cancer cells, or cancer stem cells; alone or in combination, has an increased probability of responding to treatment with the agent that inhibits or kills cancer associated mesenchymal cells, tumor initiating cancer cells, or cancer stem cell; alone or in combination, relative to a reference subject or group of subjects.

The term “location”, as used herein, refers to a zone of a sample defined by preselected criteria, such as morphology, histopathology, and other attributes. A zone of a tumor can be defined by a unique gene expression pattern of a set of preselected genes. A zone may be classified as containing a specific cell type or multiple cell types, e.g., a zone may be classified as a nodule of cancer stem cells; a nodule of cancer associated mesenchymal cells; a nodule of tumor initiating cancer cells; a zone of transition, e.g., an area between epithelial and mesenchymal features of a tumor region; or it may be a niche indicated by the presence of a particular cell type or class, e.g., mesenchymal cells, stromal cells, inflammatory cells, endothelial cells, etc.

“Unlikely to” or “decreased likelihood” refers to a decreased probability that an event, item, object, thing or person will occur with respect to a reference. Thus, a subject that is unlikely to respond to treatment with an agent that inhibits or kills cancer associated mesenchymal cells, tumor initiating cancer cells, or cancer stem cells; alone or in combination, has a decreased probability of responding to treatment with the agent that inhibits or kills cancer associated mesenchymal cells, tumor initiating cancer cells, or cancer stem cells; alone or in combination, relative to a reference subject or group of subjects.

“Sequencing” a nucleic acid molecule requires determining the identity of at least one nucleotide in the molecule. The identity of less than all of the nucleotides in a molecule can be determined. The identity of a majority or all of the nucleotides in the molecule can be determined.

The terms “sample” and “subject sample” are used interchangeably herein. These terms refer to biological material obtained from a subject. The source of the sample can be solid tissue as from a fresh, frozen and/or preserved organ, tissue sample, biopsy, or aspirate; blood or any blood constituents; bodily fluids such as cerebral spinal fluid, amniotic fluid, peritoneal fluid or interstitial fluid; or cells from any time in gestation or development of the subject. The tissue sample can contain compounds that are not naturally intermixed with the tissue in nature such as preservatives, anticoagulants, buffers, fixatives, nutrients, antibiotics or the like. The sample can be preserved as a frozen sample or as formaldehyde- or paraformaldehyde-fixed paraffin-embedded (FFPE) tissue preparation. For example, the sample can be embedded in a matrix, e.g., an FFPE block or a frozen sample. The sample can also be a cell line, a cell line previously established, a cell line derived previously from a subject, etc.

The terms “treat” and “treatment” and “treatment regimen” and “therapeutic regimen” are used interchangeably herein. As used herein, the terms “treat” and “treatment” and “treatment regimen” and “therapeutic regimen” are defined as the application or administration of a compound, alone or in combination with, a second compound to a sample, e.g., a sample, or application or administration of the compound to an isolated tissue or cell, e.g., cell line, from a subject, e.g., a subject, who has a disorder (e.g., a disorder as described herein), a symptom of a disorder, or a predisposition toward a disorder, with the purpose to cure, heal, alleviate, relieve, alter, remedy, ameliorate, improve or affect the disorder, one or more symptoms of the disorder or the predisposition toward the disorder (e.g., to minimize at least one symptom of the disorder or to delay onset of at least one symptom of the disorder).

A “weighting factor” as used herein, refers to an element used as an adjustment factor for a specific value or group of similar values.

A subject that will “respond positively” or “respond favorably” as used herein, refers to a subject that will experience some degree of alleviation in one or more characteristics of a disease or disorder after receiving treatment with a therapeutic agent; and/or some degree of alleviation in one or more symptoms caused by a disease or disorder, after receiving treatment with a therapeutic agent.

A “responder” as used herein, is a subject that will experience some degree of alleviation in one or more characteristics of a disease or disorder; and/or some degree of alleviation in one or more symptoms caused by a disease or disorder, after receiving treatment with a therapeutic agent.

A “non-responder” as used herein, is a subject that will not experience some degree of alleviation in one or more characteristics of a disease or disorder after receiving treatment with a therapeutic agent; nor some degree of alleviation in one or more symptoms caused by a disease or disorder, after receiving treatment with the therapeutic agent.

A “reference criterion” as used herein, refers to a characteristic forming the basis of comparison for the evaluation or assessment of a measured characteristic.

### Cancer and Cancer Stem Cells

Cancer is one of the most significant health conditions and leading causes of death worldwide. Currently available treatments include chemotherapy, radiation, surgery, hormonal therapy, immunotherapy, epigenetic therapy, anti-angiogenesis inhibitors, and other modalities, including targeted therapies, such as tyrosine kinase inhibitors and antibody based therapies. However, these treatments are ineffective in treating many cancers, and/or preventing reoccurrence. This ineffectiveness or unsustainability may be due, at least in part, to the innate heterogenic nature of cancer.

Cancers are known to be heterogeneous entities, with subsets of cancer cells exhibiting distinct molecular characteristics, including distinct gene expression profiles. Furthermore, cells with different molecular characteristics within the same cancer can respond differently to a single treatment. Cancer stem cells, cancer associated mesenchymal cells, and tumor initiating cancer cells, comprise a unique subpopulation of a tumor and have been identified in a large variety of cancer types. Relative to the remaining portion of the tumor, i.e., the tumor bulk, this subset of cancer cells is more tumorigenic, more slow growing or quiescent, and often more resistant to chemotherapeutic agents. Although, this subpopulation of cells constitutes only a small fraction of a tumor, these cells are thought to be responsible for cancer initiation, growth, and recurrence.

Given that currently available cancer treatments have, in large part, been designed to attack rapidly proliferating cells (i.e. those cancer cells that comprise the tumor bulk); cancer stem cells, cancer associated mesenchymal cells, and tumor initiating cancer cells, which are often slow growing, may be relatively more resistant to these treatments. Therefore, methods to identify cancer patients likely to respond positively to a treatment comprising an agent that inhibits or kills cancer associated mesenchymal cells, tumor initiating cancer cells, or cancer stem cells are needed; and can provide the basis for subsequent administration of a treatment comprising an agent that inhibits or kills cancer associated mesenchymal cells, tumor initiating cancer cells, or cancer stem cells to this candidate group of cancer patients.

The present invention provides a method of classifying subjects likely to respond to a particular therapeutic regimen for treating cancer. The method is based, at least in part, on the characterization of signals (e.g., the level of expression of a gene isoform) possessed by a candidate subject population for treatment with a preselected drug. In general, the method involves identifying differences in candidate and non-candidate subject populations, where for example, a subject population has a gene expression profile associated with a candidate or non-candidate classification. The method can further include administration of the therapeutic regimen to the candidate population based on the characterized gene expression profile.

Overall, the invention described herein features methods of evaluating and/or treating a subject, including acquiring a value or values that is a function of the level of expression of a plurality of gene isoforms from each of a plurality of genes selected from a first and/or second and/or third and/or fourth and/or fifth and/or sixth and/or eighth and/or ninth and/or tenth and/or eleventh and/or twelfth and/or thirteenth set of gene isoforms; responsive to the value or values, classifying the subject as a candidate or non-candidate for treatment with a preselected drug; optionally, further treating the subject by administering said agent that inhibits or kills cancer associated mesenchymal cells, tumor initiating cancer cells, or cancer stem cells, or withholding treatment from the subject; provided that if said agent that inhibits or kills cancer associated mesenchymal cells, tumor initiating cancer cells, or cancer stem cells is not administered, the acquisition of the subject sample or the acquisition of the value or values that is a function of the level of expression of a gene isoform comprises directly acquiring; thereby evaluating or treating the subject. In response to the value or values, the invention also features: stratification of a subject population; identification or selection of the subject as likely or unlikely to respond to a treatment; selection of a treatment; or prognostication of the time course of the disease in the subject; measurement of the response at the end of therapy and predicting the long term outcome; and/or determination of the cancer stem cell population as a predictor of response to a treatment or therapy.

### Subject Sample

The present invention features methods including, acquiring a subject sample. The terms “subject sample” and “sample” are used interchangeably herein. The subject sample can be a tissue, or bodily fluid, or bodily product. Tissue samples can include fixed, paraffin embedded, fresh, or frozen samples. For example, the tissue sample can include a biopsy, cheek swab, fine needle aspirates, large core needle biopsy, or directional vacuum assisted biopsy. Exemplary tissues include breast, brain, lung, pancreas, colon, prostate, lymph node, skin, hair follicles and nails. The tissue sample can also include a blood sample in which circulating tumor cells have been captured or isolated. Exemplary bodily fluids include blood, plasma, urine, lymph, tears, sweat, saliva, semen, and cerebrospinal fluid. Exemplary bodily products include exhaled breath.

The sample tissue, fluid, or product can be analyzed for the level of gene expression of a gene or a plurality of genes. The sample tissue, fluid or product can be analyzed for the level of gene expression of a gene or plurality of genes of a preselected signaling pathway or phenotypic pathway, e.g., a cancer stem cell phenotype, cancer associated mesenchymal cell phenotype, tumor initiating cancer cell phenotype, the epithelial to mesenchymal transition pathway, the Wnt signaling pathway, Notch pathway, or the TGFbeta signaling pathway. The sample tissue, fluid or product can be analyzed for the level of gene expression of a combination of genes from a plurality of preselected signaling or phenotypic pathways.

The tissue, fluid or product can be removed from the patient and analyzed. The evaluation can include one or more of: performing the analysis of the tissue, fluid or product; requesting analysis of the tissue fluid or product; requesting results from analysis of the tissue, fluid or product; or receiving the results from analysis of the tissue, fluid or product.

Acquisition of a Value or Values that is a Function of the Level of Expression of a Gene Isoform

The present invention features methods including, acquiring a value or values that is a function of the level of expression of a plurality of gene isoforms of a plurality of genes in a subject sample. The acquired value or values can be a function of a comparison with a reference criterion. The value or values can also be a function of the determination of whether the level of expression of a gene isoform has a preselected relationship with a reference criterion (e.g., comparing the level of gene expression, with a preselected reference criterion). The reference criterion, as used herein, refers to a characteristic forming the basis of comparison for the evaluation or assessment of a measured characteristic. The preselected reference criterion can include the level of expression of a gene isoform of a reference gene or the level of gene isoform expression of a group of reference genes (e.g., housekeeping genes). The preselected reference criterion can include the level of expression of a gene isoform of a gene from a control sample, e.g., a non-cancer sample. The appropriate reference criterion will depend on the gene or genes of which the level of expression of a gene isoform is being acquired and the sample from which the level of expression of a gene isoform of the genes was acquired from, and can be determined by one skilled in the art.

At least one or both of, acquiring a value or values that is the function of the level of expression of a gene isoform, and determining if the level of expression of a gene isoform has a preselected relationship with a reference criterion; can include one or more of: analyzing the sample, requesting analysis of the sample, requesting results from analysis of the sample, or receiving the results from analysis of the sample. Generally, analysis can include one or both of performing the underlying method (e.g., analysis of the level of gene expression) or receiving data from another who has performed the underlying method.

The acquired value or values can also be a function of a weighting factor. A weighting factor as used herein, refers to an element used to give an adjustment factor to a value. The weighting factor can be a composite weighting factor for a group of genes. For example, a first value or values that is a function of the level of expression of a plurality of gene isoforms of a plurality of genes can be a function of a weighting factor. The weighting factor can also be a specific weighting factor for a specific gene isoform that only applies to that specific gene isoform. For example, a first value or values that is a function of the level of expression of a gene isoform of a first gene can be a function of a weighting factor, and a second value or values that is a function of the level of expression of a second gene isoform of the first gene can be a function of a second weighting factor; the first and the second weighting factor can be different.

### Level of Expression of a Gene Isoform

The present invention features methods of acquiring a value or values that is a function of the level of expression of a gene isoform. The level of expression of a gene isoform can be a function of the level of expression of an alternatively spliced exon. The level of expression of a gene isoform can be a function of the level of expression of an alternatively spliced exon associated with the gene isoform. To acquire the level of expression of an alternatively spliced exon or gene isoform in a subject sample, the level of expression can be assayed, such as by measuring the level of a RNA product or protein product of the gene isoform or alternatively spliced exon. The level of expression can also be assayed by determining the activity levels of the protein (or RNA, e.g., mRNA) product of the gene isoform, e.g., transcriptional activation activity, catalytic activity, gene silencing activity, kinase activity, etc. The level of expression of an alternatively spliced exon or gene isoform can be assayed by measuring the relevant RNA product. For example, mRNA can be assayed by a PCR based method. For example, mRNA can be isolated from a tissue sample, and subjected to qRT-PCR, and, optionally, Southern blot analysis, or gene chip or microarray analysis or some variant thereof. Levels of expression of an alternatively spliced exon or gene isoform can also be assayed, for example by exon microarray with single probe set or with multiple probe sets, for each of a plurality of genes. The level of expression of an alternatively spliced exon or gene isoform can also be assayed by quantitative RNA sequencing. The sample, or the mRNA isolated from, or amplified from, the sample, can be applied to a nucleic acid microarray, or chip array, e.g., exon microarray. The level of expression of an alternatively spliced exon or gene isoform can also be assayed by detecting a protein product, e.g., an alternatively spliced protein. For example, the level of expression of an alternatively spliced protein product can be assayed using antibodies specific for the alternatively spliced protein or antibodies specific for the alternatively spliced exon, in immunohistochemistry or immunoassays, e.g., ELISA, Western blot. The level of expression of an alternatively spliced exon or gene isoform can further be assayed in specific subregions of a sample. The levels of expression of an alternatively spliced exon or gene isoform can also be measured by other molecular biology techniques known to those skilled in the art.

Optionally, the data related to the level of an alternatively spliced exon and/or gene isoform can be configured into a file, such as a data file, e.g., an image corresponding to the gene expression levels. Optionally, the data can be stored in a tangible medium and/or transmitted to a second site. The evaluation of the data file or image can include one or more of performing statistical data analysis or imaging analysis, requesting statistical data analysis or imaging analysis, requesting results from statistical data analysis or imaging analysis, or receiving the results from data statistical analysis or imaging analysis.

### Level of Gene Expression

The present invention features methods of acquiring a value or values that is a function of the level of gene expression of a plurality of genes. To acquire the level of gene expression in a subject sample, the level of gene expression can be assayed, such as by measuring the level of RNA or protein product produced by the relevant gene. Thus the level of gene expression can be a function of the level of a RNA product produced by the relevant gene; or the level of gene expression can be a function of the level of a protein product produced by the relevant gene. The level of gene expression can also be a function of the protein or RNA activity level, which can be assayed by determining the protein (or RNA, e.g., mRNA) activity levels, e.g., transcriptional activation activity, catalytic activity, gene silencing activity, kinase activity, etc. The level of RNA expression can be assayed by a PCR based method. For example, mRNA can be isolated from a tissue sample, and subjected to qRT-PCR, and, optionally, Southern blot analysis, or gene chip or microarray analysis or some variant thereof. The subject sample, or the mRNA isolated from, or amplified from, the subject sample, can be applied to a nucleic acid microarray, or chip array. The level of RNA expression can also be measured by, for example, RNA in situ hybridization, quantitative RNA sequencing, or Northern blot. The level of protein product expressed by the relevant gene can be assayed by various antibody based techniques, including but not limited to Western blot, immunohistochemistry, and immunoassays, e.g. ELISA. The levels of gene expression, e.g., level of RNA expression of the relevant gene, level of protein expression of the relevant gene; can be assayed by other molecular biology methods known to those skilled in the art.

Optionally, the level of gene expression data can be configured into a file, such as a data file, e.g., an image corresponding to the levels of gene expression. Optionally, the gene expression data can be stored in a tangible medium and/or transmitted to a second site. The evaluation of the data file or image can include one or more of, performing statistical data analysis or imaging analysis, requesting statistical data analysis or imaging analysis, requesting results from statistical data analysis or imaging analysis, or receiving the results from data statistical analysis or imaging analysis.

### Location Specific Acquisition of the Level of Gene Isoform Expression

The present invention features methods which include the acquisition of a value or values for locations in the subject sample. The value or values can be a function of the level of expression of a gene isoform of a gene, or a plurality of gene isoforms of a gene, or a plurality of gene isoforms of a plurality of genes. The value or values can be a function of the level of expression of a gene isoform of a gene, or a plurality of gene isoforms of a gene, or a plurality of gene isoforms of a plurality of genes; and further a function of the level of gene expression of a gene or a plurality of genes. This can include the acquisition of a first value or values for a first location in the subject sample, and a second value or values for a second location in the subject sample, in which the value or values are a function of the level of expression of a gene isoform of a gene, or a plurality of gene isoforms of a gene, or a plurality of gene isoforms of a plurality of genes. This can include the acquisition of a first value or values for a first location in the subject sample, and a second value or values for a second location in the subject sample, in which the value or values are a function of the level of expression of a gene isoform of a gene, or a plurality of gene isoforms of a gene, or a plurality of gene isoforms of a plurality of genes; and further a function of the level of gene expression of a gene or a plurality of genes.

The term, “location”, as used herein, refers to a zone of a sample defined by preselected criteria, such as morphology, histopathology, and other attributes. A zone of a tumor can be defined by a unique gene expression pattern of a set of preselected genes. A zone may be classified as containing specific cell type or multiple cell types, e.g., a zone may be classified as a nodule of cancer stem cells, a nodule of cancer associated mesenchymal cells, a nodule of tumor initiating cancer cells; a zone of transition, e.g., an area between epithelial and mesenchymal features of a tumor region; or a boundary between tumor regions of different types; or it may be a niche indicated by the presence of a particular cell type or class, e.g., mesenchymal cells, stromal cells, inflammatory cells, endothelial cells, cancer stem cells, cancer associated mesenchymal cells, tumor initiating cancer cells, etc.

The level of gene isoform expression and/or gene expression at a location can be measured by RNA in situ hybridization and/or antibody based immunohistochemistry techniques. These techniques also allow for the association of the levels of gene isoform expression and/or gene expression with specific cell types in a zone or region through further definition or identification of the cells. The definition or identification of these cells can be assayed using computational overlays of the cells with specific gene markers of interest, or for adjoining cells. For example, an overlay may be achieved by evaluation of serial sections of formalin-fixed or frozen tumor tissues that are sectioned 3-5 microns in thickness. Adjoining sections may be evaluated with different probes, and computational methods applied to condense into a single image file with pseudocoloring representative of the different probes. Alternatively, probes that may be identified in different wavelength channels may be used together. The definition or identification of these cells can be determined by assaying the level of expression of gene markers of interest; or assaying the level of expression of gene markers of interest in adjoining cells. The definition or identification of the cells can also be assayed by histopathology criteria, e.g., cell shape, cell size, shape of cell, nucleus shape, nucleus size, and nuclei morphology, e.g., fuzzy nuclei.

The location in the subject sample can be defined, for example, as a distance from a morphological region of the subject sample, e.g., distance from an endothelial cell or blood vessel. The location can be the whole subject sample, e.g., a tumor sample. A first location can be the whole subject sample; with subsequent acquisition of the level of gene expression of a subset of genes that define a specific zone, e.g., zones defined by biological criteria, such as detection of genes associated with a specific identity, e.g., cancer stem cell, EMT, vasculature, etc.

The acquired value or values of each location can be a function of a comparison with a reference criterion. The value or values can be a function of the level of expression of a single gene isoform at the location or a function of a combination of the level of expression of multiple gene isoforms of a gene at the location; or a combination of the level of expression of multiple gene isoforms of multiple genes at the location. For example, the level of gene isoform expression of a group of gene isoforms can be measured with a uniform technique so that the collective expression of a set of gene isoforms together is acquired. For example, RNA in situ hybridization techniques can be used in which probe sets are used for two or more gene isoforms of interest that may be combined for analysis of subject samples.

The acquired value or values can be a function of a comparison with a reference criterion. The value or values can also be a function of the determination of whether the level of gene isoform expression has a preselected relationship with a reference criterion (e.g., comparing the level of gene isoform expression, with a preselected reference criterion). The reference criterion, as used herein, refers to a characteristic forming the basis of comparison for the evaluation or assessment of measured characteristic. The preselected reference criterion can include the level of gene isoform expression of a reference gene or the level of gene isoform expression of a group of reference genes (e.g., housekeeping genes). The preselected reference criterion can include the level of gene isoform expression of a gene from a control sample, e.g., a non-cancer sample. The determination of whether the level of gene isoform expression has a preselected relationship with a reference criterion can also include comparing the acquired value or values of a first location with the acquired value or values of a second location.

At least one or both of acquiring a value or values that is the function of the level of gene isoform expression at a first and/or second location, and determining if the level of gene isoform expression has a preselected relationship with a reference criterion, can include one or more of the following: analyzing the sample; requesting analysis of the sample; requesting results from analysis of the sample; or receiving the results from analysis of the sample. Generally, analysis can include one or both of performing the underlying method (e.g., analysis of the level of gene expression) or receiving data from another who has performed the underlying method.

The value or values of a first location can be associated with a higher or lower likelihood of being a cancer stem cell, cancer associated mesenchymal cell, or tumor initiating cancer cell, than a second value or values of a second location. The value or values of a first location can be associated with a higher or lower likelihood of being a cancer stem cell than a second value or values of a second location. The value or values of a first location can be associated with a higher or lower likelihood of being a cancer associated mesenchymal cell than a second value or values of a second location. The value or values of a first location can be associated with a higher or lower likelihood of being a tumor initiating cancer cell than a second value or values of a second location. Responsive to the acquisition of the value or values acquired for each of a plurality of locations, each location can be classified as being indicative of a cancer stem cell or non-cancer stem cell. For example, a location indicative of a cancer stem cell or a tumor initiating cancer cell can exhibit a high level of CD44 gene expression (CD44(high)) and a concurrent low level of CD24 gene expression (CD24(low)) compared to a reference criterion; an increased level of gene expression compared to a reference criterion of an EMT (epithelial to mesenchymal transition) transcription factor, e.g., ZEB1, Twist, FoxC2; a decreased level of gene expression compared to a reference criterion of tight junction and adhesion genes, e.g., Claudin1-7, E-cadherin; an increased level of gene expression of mesenchymal adhesion proteins, e.g., N-cadherin. Responsive to the acquisition of the value or values acquired for each of a plurality of locations, each location can be classified as a cancer stem cell or non-cancer stem cell. Each location can also be classified as a cancer stem cell, a cancer associated mesenchymal cell, or a tumor initiating cancer cell.

Where the value or values of a location are a function of the level of gene isoform expression of multiple gene isoforms of a gene and/or multiple gene isoforms of multiple genes; the value or values can be indicative of a cancer stem cell, cancer associated mesenchymal cell, or tumor initiating cancer cell. For example, the level of gene isoform expression of a set of gene isoforms can be measured with a uniform technique as described above so that the collective level of expression of the genes identify cancer stem cells, cancer associated mesenchymal cells, or tumor initiating cancer cells. Where the value or values of a location are a function of the level of gene isoform expression of multiple gene isoforms, the value or values can be indicative of a cancer stem cell. For example, the level of gene isoform expression of a set of gene isoforms can be measured with a uniform technique as described above so that the collective level of expression of the genes identifies cancer stem cells. Where the value or values of a location are a function of the level of gene isoform expression of multiple gene isoforms, the value or values can be indicative of a cancer associated mesenchymal cell. For example, the level of gene isoform expression of a set of gene isoforms can be measured with a uniform technique as described above so that the collective level of expression of the gene isoforms identifies cancer associated mesenchymal cells. Where the value or values of a location are a function of the level of gene isoform expression of multiple gene isoforms, the value or values can be indicative of a tumor initiating cancer cell. For example, the level of gene isoform expression of a set of gene isoforms can be measured with a uniform technique as described above so that the collective level of expression of the gene isoforms identifies tumor initiating cancer cells.

The locations can be separated by no distance, i.e., adjoining locations, in the subject sample or separated by range of distances; up to the maximum distance allowed by the sample size. For example, the locations can be separated by zero microns, ten microns, twenty microns, thirty microns, forty microns, fifty microns, sixty microns, seventy microns, eighty microns, ninety microns, one hundred microns, one hundred and fifty microns, two hundred microns, or three hundred microns; the locations can be separated by more than zero microns, more than ten microns, more than twenty microns, more than thirty microns, more than forty microns, more than fifty microns, more than sixty microns, more than seventy microns, more than eighty microns, more than ninety microns, more than one hundred microns, more than one hundred and fifty microns, more than two hundred microns, or more than three hundred microns; separated by at least one micron but not over one hundred microns; separated by at least fifty microns but not over one hundred microns; separated by at least one hundred microns; separated by at least one hundred microns but not more than two hundred microns; separated by at least two hundred microns but not more than three hundred microns; separated by at least three hundred microns; separated by at least four hundred microns; separated by at least five hundred microns; separated by at least six hundred microns, separated by at least seven hundred microns, separated by at least eight hundred microns, separated by at least nine hundred microns; separated by at least one thousand microns; separated by a distance over one thousand microns; separated by a distance under one thousand microns. The distance between locations can be any distance between zero and the maximum distance two locations can be separated based on the size of the sample, including zero and the maximum distance two locations can be separated based on the size of the sample.

The average distance between the locations can be zero microns; ten microns; twenty microns; thirty microns; forty micron; fifty microns; sixty microns; seventy microns; eighty microns; ninety microns; or one hundred microns. The average distance between the locations can be more than zero microns; more than ten microns; more than twenty microns; more than thirty microns; more than forty micron; more than fifty microns; more than sixty microns; more than seventy microns; more than eighty microns; more than ninety microns; or more than one hundred microns. The average distance between the locations can be more than one thousand microns. The average distance between the locations can be more than one hundred microns; more than 200 hundred microns; more than three hundred microns; more than four hundred microns; more than five hundred microns, or more than one thousand microns. The average distance between locations can be any distance between zero and the maximum distance two locations can be separated based on the size of the sample, including zero and the maximum distance two locations can be separated based on the size of the sample.

### Gene Set Score

The present invention features methods of acquiring a gene set score. The gene set score can be a function of the level of gene expression of a plurality of genes. The level of gene expression can be acquired as described above. The gene set score can further be a function of the level expression of a gene isoform. The level of a gene isoform can be acquired as described above. The gene set score can be a function of both the level of gene expression and the level of expression of a gene isoform. The gene set score can be a function of both the level of gene expression and the level of expression of a plurality of gene isoforms of a gene. The gene set score can be a function of both the level of gene expression of a gene or plurality of genes; and the level of expression of a gene isoform of a gene. The gene set score can be a function of the level of gene expression of a gene or plurality of genes; and the level of expression of each gene isoform of a plurality of gene isoforms of a gene. The gene set score can be a function of both the level of gene expression of a gene or plurality of genes; and the level of expression of a plurality of gene isoforms of a gene. The set gene score can be a function of both the level of gene expression of a gene or plurality of genes; and the level of expression of a plurality of gene isoforms of a plurality of genes. The gene set score can be a function of both the level of gene expression of a gene or plurality of genes; and the level of expression of each gene isoform of a plurality of gene isoforms of a plurality of genes.

The gene set score can be acquired by mathematical computation. The gene set score can be computed using the following algorithm:

$${S}_{sig \_ X} = \frac{1}{N} {\sum }_{i = 1}^{N} ( {e}_{i} - {\overline{e}}_{i} )$$

### Where:

S_{sig}_{_{—}}_{X}=the score for a subset of the genes in the signature gene set (i.e., S_{sig}_{_{—}}_{UP} or S_{sig}_{_{—}}_{DN})

N=number of genes in the gene set

eᵢ=the log 2 expression level of gene in the gene set

ēᵢ=the mean log 2 expression level of gene i over all samples in the sample set

### Gene Set Score:

S_{sig}=S_{sig}_{_{—}}_{UP}−S_{sig}_{_{—}}_{DN}

### Where:

S_{sig}_{_{—}}_{UP}=gene set score over upregulated genes in the signature

S_{sig}_{_{—}}_{DN}=gene set score over downregulated genes in the signature.

### Genotype

The present invention features methods that include the acquisition of a genotype of the subject sample. The subject sample can be any sample type described herein, e.g., a tissue sample, bodily fluid, or bodily product. The genotype can be directly acquired or indirectly acquired. The genotype can be directly acquired through assaying. The genotype can be assayed using a sequencing based method. “Sequencing” a nucleic acid molecule as used herein, requires determining the identity of at least one nucleotide in the molecule. The identity of less than all of the nucleotides in a molecule can be determined. The identity of a majority or all of the nucleotides in the molecule can be determined. The genotype can be assayed using a sequencing based method, e.g., SNP (single nucleotide polymorphism) analysis, PCR based method, restriction fragment length polymorphism, terminal restriction fragment length polymorphism, amplified restriction fragment length polymorphism, multiplex restriction fragment length polymorphism, or other sequencing and molecular biology techniques known to those skilled in the art.

In genotyping, genetic events associated with cancer can be assayed. For example, nucleotides of the sample can be sequenced to determine the presence or absence of a genetic event associated with cancer; an oncogene or oncogenes and/or tumor suppressor genes can be sequenced, e.g., Abl, Af4/hrx, akt-2, alk, alk/npm, aml 1, aml 1/mtg8, APC, axl, bcl-2, bcl-3, bcl-6, bcr/abl, brca-1, brca-2, beta-catenin, CDKN2, c-myc, c-sis, dbl, dek/can, E2A/pbx1, egfr, en1/hrx, erg/TLS, erbB, erbB-2, erk, ets-1, ews/fli-1, fms, fos, fps, gli, gsp, HER2/neu, hox11, hst, IL-3, int-2, jun, kit, KS3, K-sam, Lbc, lck, lmo1, lmo2, L-myc, lil-1, lyt-10, lyt-10/C alpha1, mas, mdm-2, mll, mos, mtg8/aml1, myb, myc, MYH11/CBFB, neu, nm23, N-myc, ost, p53, pax-5, pbx1/E2A, pdgfr, PI3-K, pim-1, PRAD-1, raf, RAR/PML, rash, rasK, rasN, Rb, rel/nrg, ret, rhom1, rhom2, ros, ski, sis, set/can, src, tal1, tal2, tan-1, telomerase, Tiam1, TSC2, trk, vegfr, or wnt.

### Classification

The present invention features methods including, classifying the subject, e.g., classifying the subject as a candidate or a non-candidate for treatment with a preselected drug, e.g., an agent that inhibits or kills cancer associated mesenchymal cells, tumor initiating cancer cells, or cancer stem cells. As used herein, a subject who is a “candidate” is a one more likely to respond to a particular therapeutic regimen, relative to a reference subject or group of subjects. A “non-candidate” subject is one not more likely to respond to a particular therapeutic regimen, relative to a reference subject or group of subjects. The preselected drug can include but is not limited to, an agent that inhibits or kills cancer associated mesenchymal cells, tumor initiating cancer cells, or cancer stem cells; which can include but is not limited to, e.g., salinomycin; a gamma secretase inhibitor; a DLL4 inhibitor, e.g., a therapeutic antibody targeting DLL4; a TRAIL inhibitor, e.g., a therapeutic antibody targeting TRAIL; a Hedgehog inhibitor, e.g., a therapeutic antibody targeting Hedgehog; a NOTCH3 inhibitor, e.g., a therapeutic antibody targeting NOTCH3; a NOTCH4 inhibitor, e.g., a therapeutic antibody targeting NOTCH4; a panNOTCH inhibitor, e.g., a therapeutic antibody targeting panNOTCH; a FGFR1 inhibitor, e.g., a therapeutic antibody targeting FGR1; a FGFR2 inhibitor, e.g., a therapeutic antibody targeting FGR2; a FGFR3 inhibitor, e.g., a therapeutic antibody targeting FGR3; a FGFR4 inhibitor, e.g., a therapeutic antibody targeting FGR4; a RON inhibitor, e.g., a therapeutic antibody targeting RON; Wnt pathway inhibitor, e.g., therapeutic antibodies targeting the Wnt pathway; a PI3Kinase inhibitor; a mTOR inhibitor; sodium meta arsenite; verapail; reserpine; a perifosen inhibitor of FAK1; a FAK inhibitor; a p38 inhibitor. Classification as a candidate subject can also reflect an increased likelihood the subject will respond positively to treatment with an agent that inhibits or kills cancer associated mesenchymal cells, tumor initiating cancer cells, or cancer stem cells.

### Administration

The present invention features methods including, administering a treatment comprising an agent that inhibits or kills cancer associated mesenchymal cells, tumor initiating cancer cells, or cancer stem cells to the subject. The invention can further include selecting a regimen, e.g., dosage, formulation, route of administration, number of dosages, or adjunctive or combination therapies of an agent that inhibits or kills cancer associated mesenchymal cells, tumor initiating cancer cells, or cancer stem cells. The administration of an agent that inhibits or kills cancer associated mesenchymal cells, tumor initiating cancer cells, or cancer stem cells can be responsive to the acquisition of the value or values that is a function of the level of gene expression described herein, and/or classification of a subject as a candidate or non-candidate for treatment with an agent that inhibits or kills cancer associated mesenchymal cells, tumor initiating cancer cells, or cancer stem cells. The selection of the regimen can be responsive to the acquisition of the value or values that is a function of the level of expression of a plurality of gene isoforms described herein, and/or classification of a subject as a candidate or non-candidate for treatment with an agent that inhibits or kills cancer associated mesenchymal cells, tumor initiating cancer cells, or cancer stem cells. The invention can further include the administration of the selected regimen. The administration can be provided responsive to acquiring knowledge or information of the value or values that is a function of the level expression of a plurality of gene isoforms described herein, from another party; receiving communication of the presence of the value or values that is a function of the level expression of a plurality of gene isoforms in a subject; or responsive to the acquisition of the value or values that is a function of the level expression of a plurality of gene isoforms in a subject, wherein the acquisition arises from collaboration with another party.

An agent that inhibits or kills cancer associated mesenchymal cells, tumor initiating cancer cells, or cancer stem cells, e.g., salinomycin; a gamma secretase inhibitor; a DLL4 inhibitor, e.g., a therapeutic antibody targeting DLL4; a TRAIL inhibitor, e.g., a therapeutic antibody targeting TRAIL; a Hedgehog inhibitor, e.g., a therapeutic antibody targeting Hedgehog; a NOTCH3 inhibitor, e.g., a therapeutic antibody targeting NOTCH3; a NOTCH4 inhibitor, e.g., a therapeutic antibody targeting NOTCH4; a panNOTCH inhibitor, e.g., a therapeutic antibody targeting panNOTCH; a FGFR1 inhibitor, e.g., a therapeutic antibody targeting FGR1; a FGFR2 inhibitor, e.g., a therapeutic antibody targeting FGR2; a FGFR3 inhibitor, e.g., a therapeutic antibody targeting FGR3; a FGFR4 inhibitor, e.g., a therapeutic antibody targeting FGR4; a RON inhibitor, e.g., a therapeutic antibody targeting RON; Wnt pathway inhibitor, e.g., therapeutic antibodies targeting the Wnt pathway; a PI3Kinase inhibitor; a mTOR inhibitor; sodium meta arsenite; verapail; reserpine; a perifosen inhibitor of FAK1; a FAK inhibitor; a p38 inhibitor; can be administered to a subject using any amount and any route of administration effective for treating cancer, or symptoms associated with cancer. The exact dosage required will vary from subject to subject, depending on subject specific factors, e.g., the age and general condition of the subject, concurrent treatments, concurrent diseases or conditions; cancer specific factors, e.g., the type of cancer, whether the cancer is recurrent, whether the cancer is metastatic, the severity of the disease; and agent specific factors., e.g., its composition, its mode of administration, its mode of activity, and the like. For example, the dosage may vary depending on whether the subject is currently receiving or had previously received a treatment regimen prior to the administration of an agent that inhibits or kills cancer associated mesenchymal cells, tumor initiating cancer cells, or cancer stem cells; whether the subject is a non-responder to such current or previous treatment; whether the subject's cancer is recurrent; or whether the subject's cancer has metastasized to a second tissue site.

The total daily usage of a therapeutic composition of an agent that inhibits or kills cancer associated mesenchymal cells, tumor initiating cancer cells, or cancer stem cells can be decided by an attending physician within the scope of sound medical judgment. The specific therapeutically effective, dose level for any particular subject will depend upon a variety of factors including the type of cancer being treated; the severity of the cancer; the metastatic state of the cancer; the recurrence state of the cancer; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The agent that inhibits or kills cancer associated mesenchymal cells, tumor initiating cancer cells, or cancer stem cells may be administered by any route, including by those routes currently accepted and approved for known products. Exemplary routes of administration include, e.g., oral, intraventricular, transdermal, rectal, intravaginal, topical (e.g. by powders, ointments, creams, gels, lotions, and/or drops), mucosal, nasal, buccal, enteral, vitreal, sublingual; by intratracheal instillation, bronchial instillation, and/or inhalation; as an oral spray, nasal spray, and/or aerosol, and/or through a portal vein catheter. An agent may be administered in a way, which allows the agent to cross the blood-brain barrier, vascular barrier, or other epithelial barrier.

Other exemplary routes include administration by a parenteral mode (e.g., intravenous, subcutaneous, intraperitoneal, or intramuscular injection). The phrases “parenteral administration” and “administered parenterally” as used herein mean modes of administration other than enteral and topical administration, usually by injection, and include, without limitation, intravenous, intramuscular, intraarterial, intrathecal, intracapsular, intramedullary, intratumoral, intraorbital, intracardiac, intradermal, intraperitoneal, transtracheal, subcutaneous, subcuticular, intraarticular, subcapsular, subarachnoid, intraspinal, epidural and intrasternal injection and infusion.

Pharmaceutical compositions can be formulated in a variety of different forms, such as liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. A pharmaceutical composition comprising an agent that inhibits or kills cancer associated mesenchymal cells, tumor initiating cancer cells, or cancer stem cells may be administered on various dosing schedules. The dosing schedule will be dependent on several factors including, the type of cancer being treated; the severity of the cancer; the metastatic state of the cancer; the recurrence state of the cancer; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Exemplary dosing schedules of an agent that inhibits or kills cancer associated mesenchymal cells, tumor initiating cancer cells, or cancer stem cells composition include, once daily, or once weekly, or once monthly, or once every other month. The composition can be administered twice per week or twice per month, or once every two, three or four weeks. The composition can be administered as two, three, or more sub-doses at appropriate intervals throughout the day or even using continuous infusion or delivery through a controlled release formulation. In that case, the therapeutic agent contained in each sub-dose may be correspondingly smaller in order to achieve the total daily dosage. The dosage can also be compounded for delivery over several days, e.g., using a conventional sustained release formulation, which provides sustained release of the agent over a several day period. Sustained release formulations are well known in the art and are particularly useful for delivery of agents at a particular site.

The present invention features methods in which a value or values that is a function of the level of expression of a plurality of gene isoforms can be acquired at the time of or after diagnosis of cancer in a subject. The acquisition of the value or values that is a function of the level of gene expression can be acquired at a predetermined interval, e.g., a first point in time and at least at a subsequent point in time. The cancer can include cancers characterized as comprising cancer stem cells, cancer associated mesenchymal cells, or tumor initiating cancer cells. The cancer can include cancers that have been characterized as being enriched with cancer stem cells, cancer associated mesenchymal cells, or tumor initiating cancer cells. Exemplary cancers include epithelial cancers, breast, lung, pancreatic, colorectal, prostate, head and neck, melanoma, acute myelogenous leukemia, and glioblastoma. Exemplary breast cancers include triple negative breast cancer, basal-like breast cancer, claudin-low breast cancer, invasive, inflammatory, metaplastic, and advanced Her-2 positive or ER-positive cancers resistant to therapy. Other cancers include but are not limited to, brain, abdominal, esophagus, gastrointestinal, glioma, liver, tongue, neuroblastoma, osteosarcoma, ovarian, retinoblastoma, Wilm's tumor, multiple myeloma, skin, lymphoma, blood, retinal, acute lymphoblastic leukemia, bladder, cervical, kidney, endometrial, meningioma, lymphoma, skin, uterine, lung, non small cell lung, nasopharyngeal carcinoma, neuroblastoma, solid tumor, hematologic malignancy, leukemia, squamous cell carcinoma, testicular, thyroid, mesothelioma, brain vulval, sarcoma, intestine, oral, T cell leukemia, endocrine, salivary, spermatocytic seminoma, sporadic medulalry thyroid carcinoma, non-proliferating testes cells, cancers related to malignant mast cells, non-Hodgkin's lymphoma, and diffuse large B cell lymphoma.

The cancer can be a primary tumor, i.e., located at the anatomical site of tumor growth initiation. The cancer can also be metastatic, i.e., appearing at least a second anatomical site other than the anatomical site of tumor growth initiation. The cancer can be a recurrent cancer, i.e., cancer that returns following treatment, and after a period of time in which the cancer was undetectable. The recurrent cancer can be anatomically located locally to the original tumor, e.g., anatomically near the original tumor; regionally to the original tumor, e.g., in a lymph node located near the original tumor; or distantly to the original tumor, e.g., anatomically in a region remote from the original tumor.

The acquisition of a value or values that is a function of the level expression of a plurality of gene isoforms described herein, can be acquired prior to, during, or after administration of a treatment to a subject. The treatment can include an agent that inhibits or kills cancer associated mesenchymal cells, tumor initiating cancer cells, or cancer stem cells therapy. The treatment can include a chemotherapeutic agent, antiemetic, analgesic, or anti-inflammatory agent. Suitable chemotherapeutic agents are any chemical substances or compounds, such as cytotoxic or cytostatic agent, that is used to treat a condition, particularly cancer, including, but not limited to: alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, vinblastine, and paclitaxel (Taxol)); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide). Exemplary chemotherapeutic agents include, Capecitabine, Carboplatin, Cisplatin, Cyclophosphamide, Docetaxel, Doxorubicin, Epirubicin, Eribulin, mesylate5-Fluorouracil, Gemcitabine, Ixabepilone, Liposomal doxorubicin, Methotrexate, Paclitaxel, or Vinorelbine; or any combination thereof.

The subject can be a responder or non-responder to the current or prior treatment. The agent that inhibits or kills cancer associated mesenchymal cells, tumor initiating cancer cells, or cancer stem cells; can be administered as an additional therapeutic agent, e.g., an agent that inhibits or kills cancer associated mesenchymal cells, tumor initiating cancer cells, or cancer stem cells in addition to a current therapeutic regimen, or in addition to a new therapeutic regimen. The current treatment of the subject can be stopped and replaced with treatment an agent that inhibits or kills cancer associated mesenchymal cells, tumor initiating cancer cells, or cancer stem cells. The current treatment regimen can also be altered with the addition of an agent that inhibits or kills cancer associated mesenchymal cells, tumor initiating cancer cells, or cancer stem cells as an additional therapeutic agent. Therapeutic agents administered in combination with an agent that inhibits or kills cancer associated mesenchymal cells, tumor initiating cancer cells, or cancer stem cells; can kill or inhibit the growth of non-cancer stem cells, non-cancer associated mesenchymal cells, or non-tumor initiating cells in the subject.

### Kits or Products

The present invention features a kit or product that includes a means to assay the level of expression of a plurality of gene isoforms of a gene or plurality of genes in Table 1, Table 2, Table 3, Table 4, Table 5, Table 6, Table 8, Table 9, Table 10, Table 11, Table 12, and/or Table 13. For example, the kit or product can include an agent capable of interacting with a gene expression product of a gene from the genes in Table 1, Table 2, Table 3, Table 4, Table 5, Table 6, Table 8, Table 9, Table 10, Table 11, Table 12, and/or Table 13; and can further contain a second agent capable of interacting with a different gene expression product from a gene in Table 1, Table 2, Table 3, Table 4, Table 5, Table 6, Table 8, Table 9, Table 10, Table 11, Table 12, and/or Table 13. The kit can contain a plurality of different agents capable of interacting with a plurality of genes expression products from a gene in Table 1, Table 2, Table 3, Table 4, Table 5, Table 6, Table 8, Table 9, Table 10, Table 11, Table 12, and/or Table 13. The kit can contain a plurality of different agents capable of interacting with a plurality of genes expression products from a plurality of genes in Table 1, Table 2, Table 3, Table 4, Table 5, Table 6, Table 8, Table 9, Table 10, Table 11, Table 12, and/or Table 13. The agent can include, but is not limited to, an antibody, a plurality of antibodies, an oligonucleotide, or a plurality of oligonucleotides. The kit or product can further comprise an agent capable of interacting with a gene expression product of a gene not in Table 1. The kit or product can contain a plurality of agents capable of interacting with a plurality of gene expression product of a plurality of genes not in Table 1, Table 2, Table 3, Table 4, Table 5, Table 6, Table 8, Table 9, Table 10, Table 11, Table 12, and/or Table 13. The gene expression product can include, but is not limited to, a RNA product of the associated gene, or a protein product of the associated gene.

The kit or product can further optionally include reagents for performing the level of gene expression assays described herein. For example, the kit can include buffers, solvents, stabilizers, preservatives, purification columns, detection reagents, and enzymes, which may be necessary for isolating nucleic acids from a subject sample, amplifying the samples, e.g., by qRT-PCR, and applying the samples to the agent described above; or for isolating proteins from a subject sample, and applying the samples to the agent described above; or reagents for directly applying the subject sample to the agent described above. A kit can also include positive and negative control samples, e.g., control nucleic acid samples (e.g., nucleic acid sample from a non-cancer subject, or a non-tumor tissue sample, or a subject who has not received treatment for cancer, or other test samples for testing at the same time as subject samples. A kit can also include instructional material, which may provide guidance for collecting and processing patient samples, applying the samples to the level of gene expression assay, and for interpreting assay results.

The components of the kit can be provided in any form, e.g., liquid, dried, semi-dried, or in lyophilized form, or in a form for storage in a frozen condition. Typically, the components of the kit are provided in a form that is sterile. When reagents are provided in a liquid solution, the liquid solution generally is an aqueous solution, e.g., a sterile aqueous solution. When reagents are provided in a dried form, reconstitution generally is accomplished by the addition of a suitable solvent. The solvent, e.g., sterile buffer, can optionally be provided in the kit.

The kit can include one or more containers for the kit components in a concentration suitable for use in the level of gene expression assays or with instructions for dilution for use in the assay. The kit can contain separate containers, dividers or compartments for the assay components, and the informational material. For example, the positive and negative control samples can be contained in a bottle or vial, the clinically compatible classifier can be sealed in a sterile plastic wrapping, and the informational material can be contained in a plastic sleeve or packet. The kit can include a plurality (e.g., a pack) of individual containers, each containing one or more unit forms (e.g., for use with one assay) of an agent. The containers of the kits can be air tight and/or waterproof. The container can be labeled for use.

The kit can include informational material for performing and interpreting the assay. The kit can also provide guidance as to where to report the results of the assay, e.g., to a treatment center or healthcare provider. The kit can include forms for reporting the results of a gene activity assay described herein, and address and contact information regarding where to send such forms or other related information; or a URL (Uniform Resource Locator) address for reporting the results in an online database or an online application (e.g., an app). In another embodiment, the informational material can include guidance regarding whether a patient should receive treatment with an ant-cancer stem cell agent, depending on the results of the assay.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as computer readable material, video recording, or audio recording. The informational material of the kit can be contact information, e.g., a physical address, email address, website, or telephone number, where a user of the kit can obtain substantive information about the gene activity assay and/or its use in the methods described herein. The informational material can also be provided in any combination of formats.

A subject sample can be provided to an assay provider, e.g., a service provider (such as a third party facility) or a healthcare provider that evaluates the sample in an assay and provides a read out. For example, an assay provider can receive a sample from a subject, such as a tissue sample, or a plasma, blood or serum sample, and evaluate the sample using an assay described herein, and determines that the subject is a candidate to receive an agent that inhibits or kills cancer associated mesenchymal cells, tumor initiating cancer cells, or cancer stem cells.

The assay provider can inform a healthcare provider that the subject is a candidate for treatment with an agent that inhibits or kills cancer associated mesenchymal cells, tumor initiating cancer cells, or cancer stem cells, and the candidate is administered the agent that inhibits or kills cancer associated mesenchymal cells, tumor initiating cancer cells, or cancer stem cells. The assay provider can provide the results of the evaluation, and optionally, conclusions regarding one or more of diagnosis, prognosis, or appropriate therapy options to, for example, a healthcare provider, or patient, or an insurance company, in any suitable format, such as by mail or electronically, or through an online database. The information collected and provided by the assay provider can be stored in a database.

### Reports

The present invention features optionally providing a report. The report can include a prediction of the likelihood that a subject will respond positively or will not respond positively to treatment with an agent that inhibits or kills cancer associated mesenchymal cells, tumor initiating cancer cells, or cancer stem cells, e.g., salinomycin; a gamma secretase inhibitor; a DLL4 inhibitor, e.g., a therapeutic antibody targeting DLL4; a TRAIL inhibitor, e.g., a therapeutic antibody targeting TRAIL; a Hedgehog inhibitor, e.g., a therapeutic antibody targeting Hedgehog; a NOTCH3 inhibitor, e.g., a therapeutic antibody targeting NOTCH3; a NOTCH4 inhibitor, e.g., a therapeutic antibody targeting NOTCH4; a panNOTCH inhibitor, e.g., a therapeutic antibody targeting panNOTCH; a FGFR1 inhibitor, e.g., a therapeutic antibody targeting FGR1; a FGFR2 inhibitor, e.g., a therapeutic antibody targeting FGR2; a FGFR3 inhibitor, e.g., a therapeutic antibody targeting FGR3; a FGFR4 inhibitor, e.g., a therapeutic antibody targeting FGR4; a RON inhibitor, e.g., a therapeutic antibody targeting RON; Wnt pathway inhibitor, e.g., therapeutic antibodies targeting the Wnt pathway; a PI3Kinase inhibitor; a mTOR inhibitor; sodium meta arsenite; verapail; reserpine; a perifosen inhibitor of FAK1; a FAK inhibitor; a p38 inhibitor. The report can include a prediction of the likelihood a subject will respond positively or not to treatment with an agent that inhibits or kills cancer associated mesenchymal cells, tumor initiating cancer cells, or cancer stem cells. The report can also include a proposal including any one of or combination of the following: whether a subject is a candidate for treatment with an agent that inhibits or kills cancer associated mesenchymal cells, tumor initiating cancer cells, or cancer stem cells; whether a subject should be treated with a preselected drug, e.g. an agent that inhibits or kills cancer associated mesenchymal cells, tumor initiating cancer cells, or cancer stem cells; or whether treatment with a preselected drug, e.g., an agent that inhibits or kills cancer associated mesenchymal cells, tumor initiating cancer cells, or cancer stem cells, should be withheld.

The report can be provided by an assay service provider (such as a third party facility) that evaluates the sample in an assay and provides a report, or a healthcare provider. In the former case, the assay service provider can inform a healthcare provider that the subject is a candidate for treatment with an agent that inhibits or kills cancer associated mesenchymal cells, tumor initiating cancer cells, or cancer stem cells, and the candidate is administered the agent that inhibits or kills cancer associated mesenchymal cells, tumor initiating cancer cells, or cancer stem cells. The assay provider can provide the results of the evaluation, and optionally, conclusions regarding one or more of diagnosis, prognosis, or appropriate therapy options to, for example, a healthcare provider, or subject, or an insurance company, in any suitable format, such as by mail or electronically, or through an online database. The information collected and provided by the assay provider can be stored in a database. The report can be reported back to the healthcare provider, such as through a form, which can be submitted by mail or electronically (e.g., through facsimile or e-mail) or through an on-line database or on-line application (e.g., through an “app”). The results of the assay (including the level of gene expression) can be stored in a database and can be accessed by a healthcare provider, such as through the worldwide web.

### EXAMPLES

### Example 1

### The Skipped Exon Selection Method

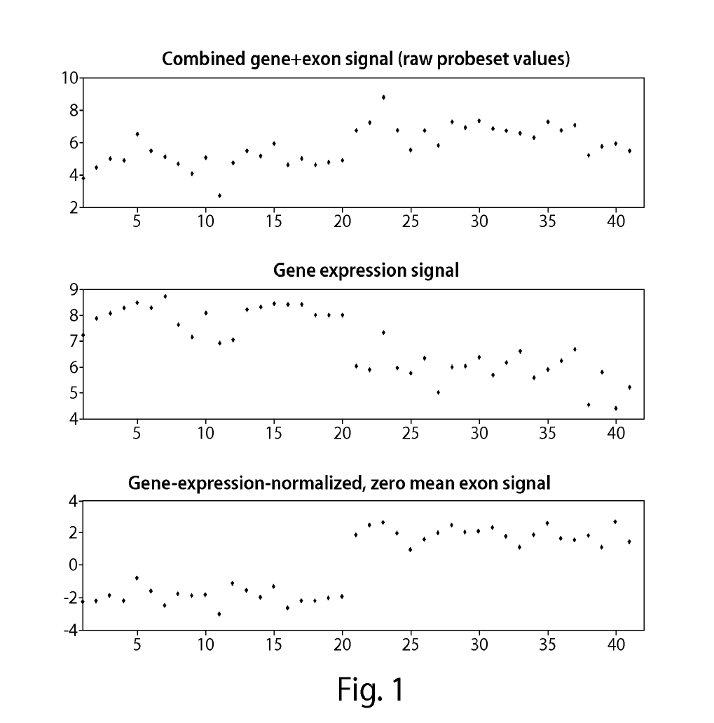

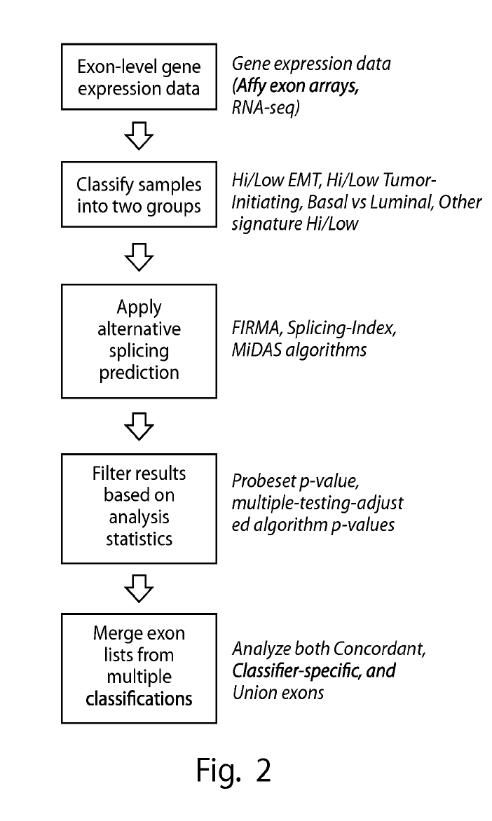

The human transcriptome is composed of transcribed genes and their various isoforms. The skipped exon selection method is based on the principal that gene regulation at the exon level may be important for cancer stem cell biology, epithelial-mesenchymal transitions (EMT) and its effects, and tumor initiating phenotypes. The method evaluates the differential expression of different isoforms by evaluating different samples or specimens (FIG. 1 and FIG. 2). Gene expression data is acquired per sample utilizing many platforms (examples include, Affymetric exon array profiles, or RNASeq). In a stepwise manner, a classification method is applied to determine two sample groups. An alternative splicing predictor algorithm (FIRMA, Splicing Index) is applied and output results are filtered with analysis statistics (probeset p-values, multiple testing adjusted algorithm p-values, and FDR). Exon lists are formed adhering to the statistical filters, and candidate probeset/exons are converted to classifier groups. The raw probeset expression values are processed from the microarray and assembled into probeset groups based on genomic structure. In order to determine differential expression, the change in gene expression between two sample sets or groups must be accounted for. Therefore the normalized change in expression for exons must exceed that for the genes. Every gene is accounted for in a similar way and the gene expression normalized zero mean exon expression level is computed. FIG. 1 illustrates the differential expression of particular exons identified.

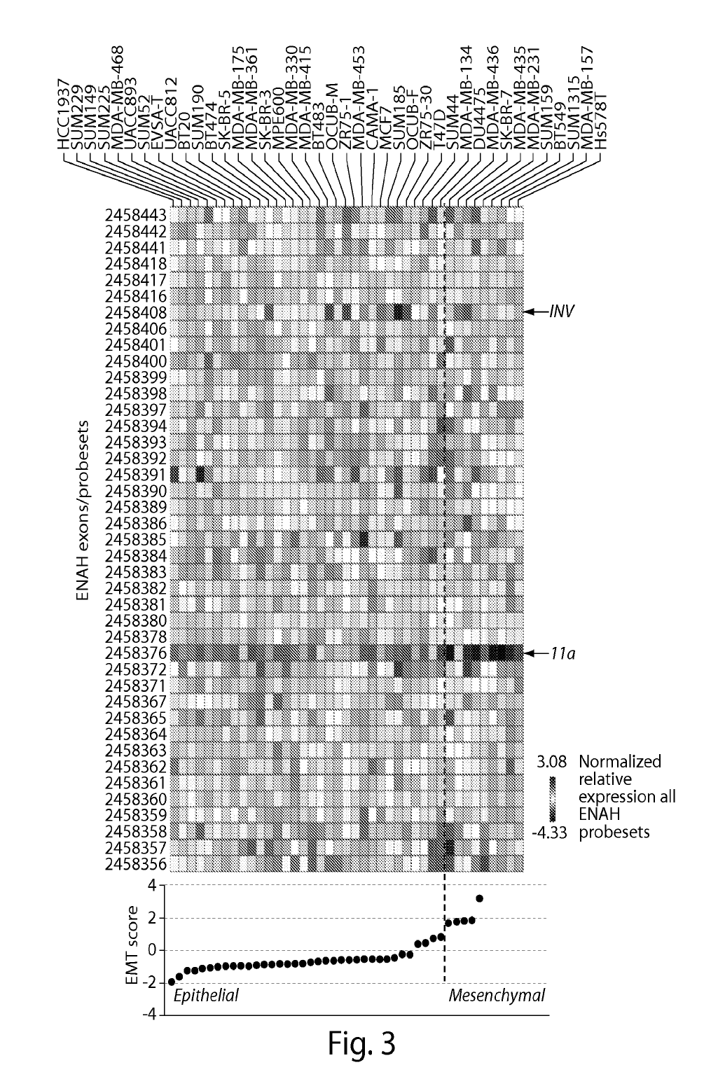

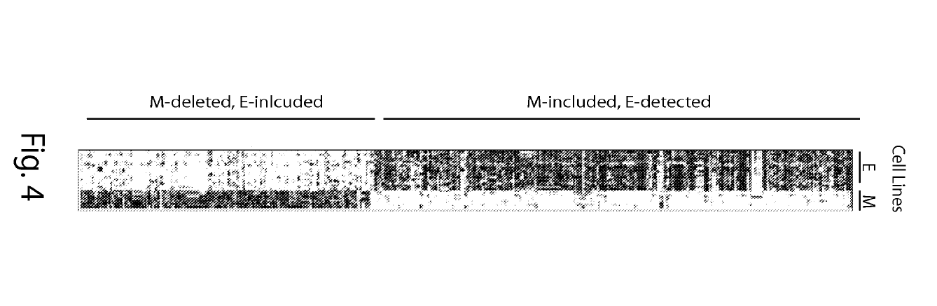

The method is exemplified by observing the different exons in one gene, where that gene may be important for cancer stem cell biology, epithelial-mesenchymal transitions and its effects, and tumor initiating phenotypes. Also, the method is useful to associate distinguishing morphologies that identify one tumor type versus another. An example is in building the distinction between basal-B and luminal subtypes in breast cancers. FIG. 3 illustrates the method of using exon probesets for a single gene ENAH, (hMENA). The top panel of the figure indicates the relative expression level of different exon probesets of ENAH based on the colorization index on the right. In this example, the normalized relative expression level of all ENAH probesets (listed on left, ENAH exons/probesets with numeric values representing genomic position) was determined to vary between 3.08 and −4.33. The bottom panel of the figure illustrates a gene set score ranking strategy applied to the exon probesets of ENAH. Different gene set score ranking criteria may also be applied.

The output of the skipped exon method indicates that the relative exon expression of the different exons of a single gene may be evaluated as a group. It is striking that whereas many of exon-based probesets demonstrate relatively little variation across breast cancer cell lines, there are particular probesets of highlighted significance. In this example the 11a exon (ENAH gene isoform containing 11a) is expressed in a pattern resembling the trend from high to low in EMT gene set scoring. The EMT gene set score is utilized and refers to the EMT gene set score formed for 41 human breast cancer cell lines as labeled in the x-axis. EMT gene set scores range from 5 to −5 in this example. The dotted Line delineates an arbitrary distinguisher between cell lines, leftward are more epithelial-like (EMT<0), and rightward cell lines that are more mesenchymal-like (EMT>0). In contrast, a separate exon in ENAH, termed INV (ENAH INV gene isoform), has slight increases in expression in certain mesenchymal cell lines, but to a lesser extent. Thus the execution of the exon discriminator profiling and classifier is a means to select probesets, exons, and gene isoforms that are candidates for differential expression between cells of different phenotypes. Single probesets may be viewed as an individual element of a larger signature.

### Example 2

### Epithelial-Mesenchymal Transition Discriminator for Breast Cancer Classification

The epithelial to mesenchymal transition (EMT) of cells in cancers has previously been highlighted by cell differentiation changes in tumors. EMT signatures of differential splicing where a change in the pattern of splicing is indicative of the epithelial to mesenchymal process relevant to the cancer progression, maintenance, differentiation, de-differentiation, transition, interaction with other cell types, metastasis, micro-colonization, tumor dormancy, tumor growth, and the like, is anticipated to be valuable to discover. A pattern or classifier may be established by discovery of exons from the same gene, or by exons of different genes with a similar pattern, whereas exons elsewhere in the same gene and in different genes may adhere to an alternatively patterned classifier. Although a single classifier portraying an alternatively spliced exon of one type is valuable, additional information may be gained by analysis of multiple types.

In this method, a group of samples is evaluated for whole transcriptome profiling using measurements of exons via probesets on microarray chips, Q-PCR, and or RNA sequencing strategies. Under these circumstances abundances of each exon are determined. The samples are ordered by a classification schema. In this case, the classification is implemented by determination of an EMT gene set score as defined by the selection of combinations of genes that are either up- or down-regulated. Each sample is assigned an EMT gene set score on an arbitrary scale but the ranking determines the degree of similarity or dissimilarity between members. In this example, 41 human breast cancer cell lines were determined to have an EMT gene set score ranging from high values in the spectrum coinciding with cell lines in the group having an EMT gene signature positivity (mesenchymal-like features of cells), and low values in the scoring associated with other cell lines having EMT gene signature negativity (epithelial-like features of cells). Cell lines that were used were derived from human breast cancers, and represented different subtypes and morphologies of the disease. Cell lines used were AU565, BT_{—}549, BT20, BT474, BT483, CAL-120, CAL-148, CAL-51, CAL85-1, CAMA-1, DU4475, EFM19, EFM-192A, EVSA-T, HBL100, HCC38, HCC70, HCC1143, HCC1395, HCC1419, HCC1428, HCC1500, HCC1569, HCC1806, HCC1937, HCC1954, HCC202, HCC2218, HDQ-P1, Hs578T, JIMT-1, KPL1, KPL4, MCF7, MDA_MB_{—}231, MDA-MB-134VI, MDA-MB-157, MDA-MB-175VII, MDA-MB-175VIII, MDA-MB-330, MDA-MB-361, MDA-MB-415, MDA-MB-435s, MDA-MB-436, MDA-MB-453, MDA-MB-468, MFM-223, MPE600, MX1, OCUB-F, OCUB-M, SK-BR-3, SK-BR-5, SK-BR-7, SUM1315, SUM149, SUM159, SUM185, SUM190, SUM225, SUM229, SUM44, SUM52, SW527, T47D, UACC-812, UACC-893, ZR75-1, ZR75-30. Other cell lines may be added based on breast cancers, or from myofibroblast or fibroblast types.

Exon microarray data collected from the cell lines listed above were analyzed using the FIRMA algorithm (as implemented by AltAnalyze) to determine which exons were differentially expressed. The FIRMA algorithm takes a set of raw microarray data (CEL files), splits the raw data into two classes, and determines which exons are most differentially expressed at a statistically significant level between the two classes. The AltAnalyze ouput files contain information on the degree of expression difference (fold-change) and several statistical measurements of the significance of the expression difference. In addition, for each exon, a measurement of the differential expression of the gene containing that exon is also provided. Exons were disregarded in subsequent analysis if the probeset p-value (a measurement of the confidence of the underlying exon expression measurement) was greater than 0.05. Exons for which the FIRMA p-value (a measurement of the exon differential expression) was greater than 0.05 were also disregarded. Finally, exons for which the differential expression of the gene containing the exon was greater than three-fold were also disregarded. The threshold for this final filtering step is arbitrary, and its main purpose is to remove exons for which the simple measurement of the overall gene expression level would be just as effective as the more difficult measurement of the exon expression difference. Therefore, the thresholds may be modulated in different ways to influence the list size of exon probesets outputted.

In the method described in this example, the FIRMA algorithm was conducted by requiring that the input data be separated into two classes, such that exons that are differentially expressed at a statistically significant level are determined between these two classes. For the EMT-score-based classification, the EMT gene set score was computed for each cell line, and a subset of the cell lines were classified as EMT-high (having an EMT score greater than zero) or EMT-low (the lowest-scoring cell lines). The cell lines in each class were:
a. EMT-high: BT_{—}549, MDA-MB-436, MDA-MB-157, CAL-120, SUM1315, SUM159, Hs578T, HCC1395, MDA_MB_{—}231
b. EMT-low: SUM149, HCC1954, BT474, HCC70, ZR75-1, MDA-MB-468, JIMT-1, EFM-192A, HCC1806

In this method, the expression level of genes (RNA expression) may be compiled and used to filter the output of alternatively spliced exons (gene isoforms). In this regard, filters of expression level differences between samples may be set to evaluate change in exon RNA abundances only above the change observed by RNA expression. Likewise, filters of exon RNA abundance between 41 breast cancer cell lines may be set to vary at up to 8-fold variation.

Optionally, the filter of exon RNA abundances may be set to vary at up to 3-fold variation, or at up to 2-fold variation. Differential exon abundance levels is therefore metered both by exon RNA expression maximal changes between subgroups, and by the relative values that are observed and present above and beyond the potential RNA expression differences. For example, if the differential exon RNA abundance is set at <2-fold, all probe set variations for every gene must not exceed a 2-fold variation between the classifier subgroups in the high EMT (mesenchymal-like) set versus the low EMT (epithelial-like) set.

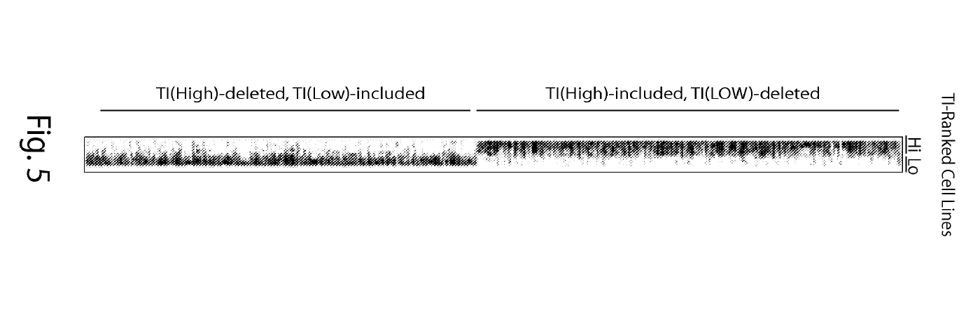

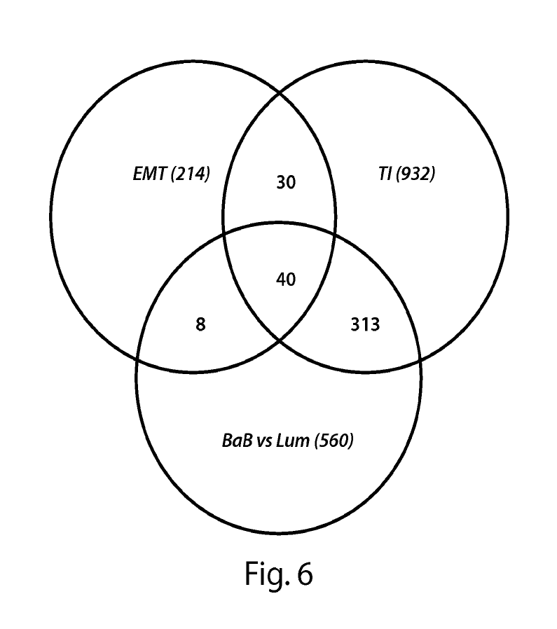

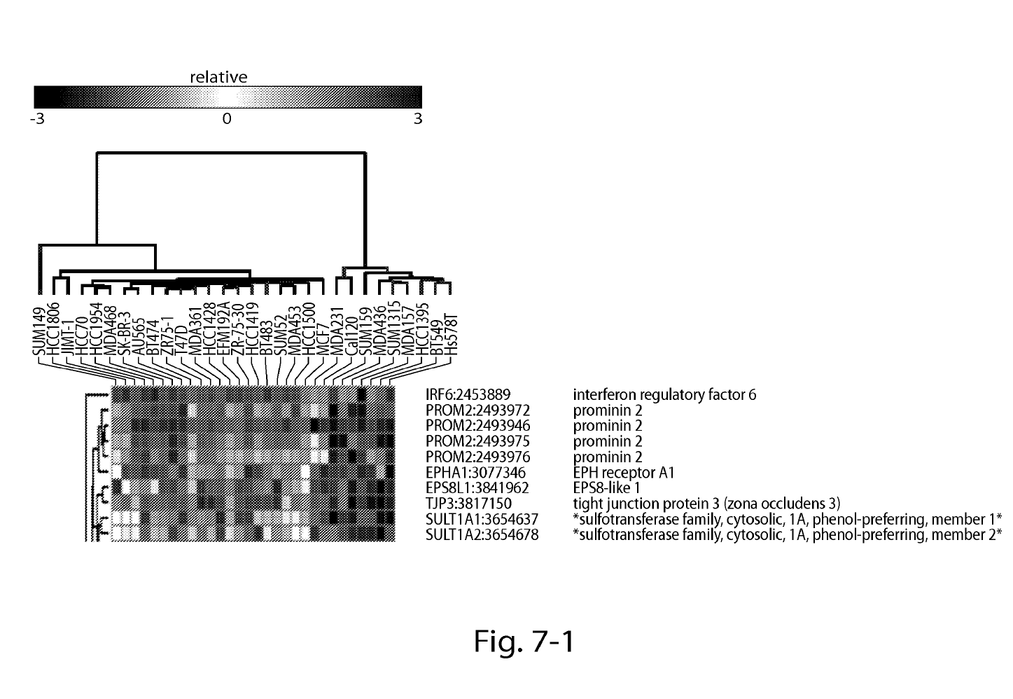

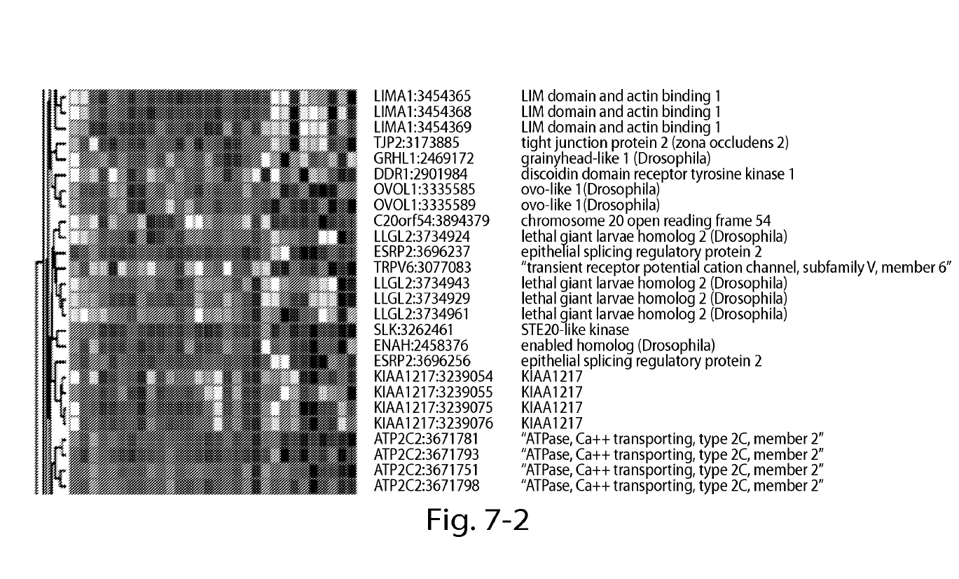

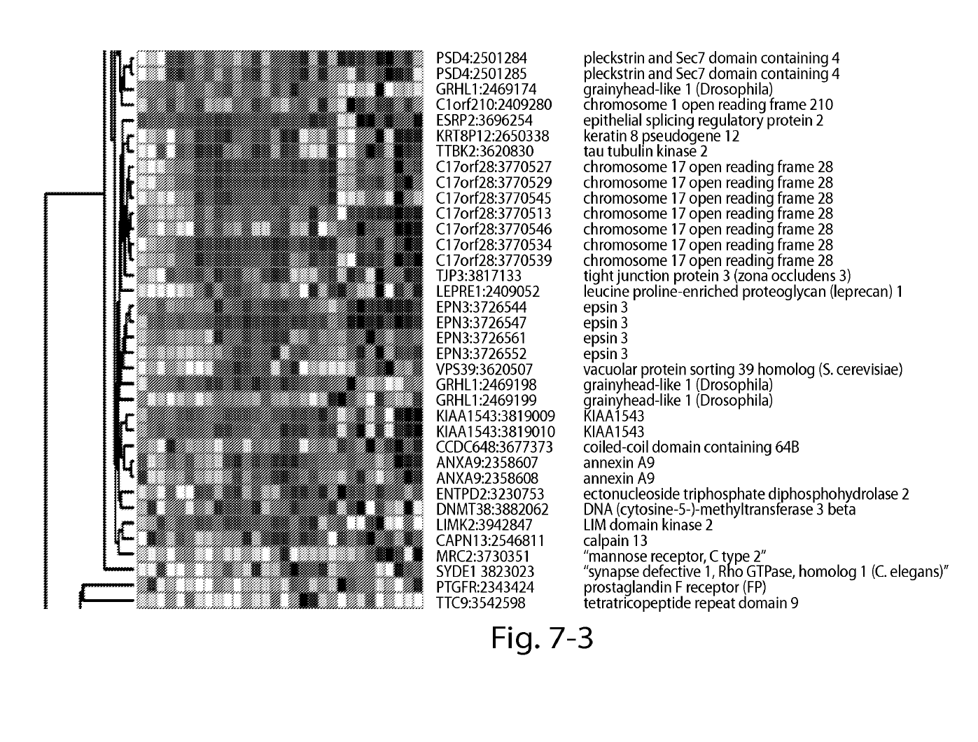

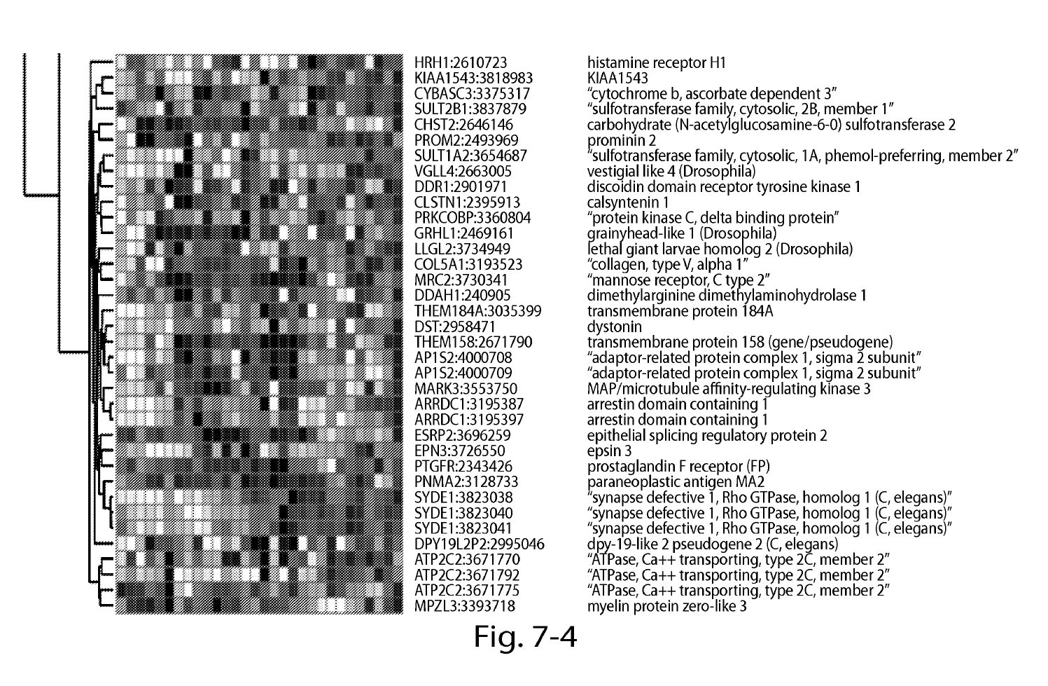

The EMT trained discriminator creates differentially expressed exons that can be ranked and compared with one another (FIG. 4). In this example, 214 exon probesets were outputted from the EMT discriminator using the E-high (epithelial-high) versus M-high (mesenchymal-high) cell line groups. As FIG. 4 illustrates, exon probesets are ordered based on similarity and two patterns emerge. First, approximately half of the probesets are indicative of a pattern that is M-high coincident with increased expression of the included exon designated by the probeset (M-included). Second, the other half of the probesets are indicative of a pattern that is E-high coincident with increased expression of the included exon designated by the probeset (E-included). These attributes define single exon probesets, groups of probesets identifying single exons, and multiple exons from many genes that may be used in identifying a similar feature from cell lines and tumors.

Upon execution of the method, gene isoforms represented by alternatively spliced exons that are measured by exon-specific probesets are evaluated, and a range of outputs is developed that have maximal to minimal differences in abundances for every probe set. An alternative splicing predictor is implemented (FIRMA, splicing index, and MiDAS algorithms). Also, exon abundance variations may be set at up to 8-fold (<8-fold) variation, or optionally may be set at up to 3-fold (<3-fold) variation. The visualization of the expression pattern of these probesets amongst all the samples (41 breast cancer cell lines) illustrates that the group of probesets defining cells with a high EMT classification are composed of classes of alternatively spliced exons that are included and others that are excluded in these cells. A tabulation of the complete EMT probesets is presented in Table 1 and Table 2. Thus, both Gene isoforms that are increased in expression and others that are reduced in expression may contribute to defining cells with the EMT features.

### Example 3

### Tumor Initiating Cell Discriminator for Breast Cancer Classification

Tumor initiating (TI) cells of cancers are identified by signatures of differential splicing, where changes in the pattern of splicing is indicative of a biological process relevant to the cancer progression, maintenance, differentiation, de-differentiation, transition, interaction with other cell types, metastasis, micro-colonization, tumor dormancy, tumor growth, and the like. A pattern or classifier may be established by discovery of exons from the same gene, or by exons of different genes with a similar pattern, whereas exons elsewhere in the same gene and in different genes may adhere to an alternatively patterned classifier. Although a single classifier portraying an alternatively spliced exon of one type is valuable, additional information may be gained by analysis of multiple types.

In the method, a group of samples is evaluated for whole transcriptome profiling using measurements of exons via probesets on microarray chips, Q-PCR, and or RNA sequencing strategies. Under these circumstances abundances of each exon are determined. The samples are ordered by a classification schema. In this case, the classification is implemented by determination of a tumor initiating gene set score as defined by the selection of combinations of genes that are either up- or down-regulated. Each sample is assigned a tumor initiating gene set score on an arbitrary scale. In this example, 41 human breast cancer cell lines were determined to have a TI gene set score ranging from high values in the spectrum coinciding with cell lines in the group having tumor initiating gene signature positivity, and low values in the scoring associated with other cell lines having tumor initiating gene signature negativity,

In the method, the expression level of genes (RNA expression) may be compiled and used to filter the output of alternatively spliced exons (gene isoforms). In this regard, filters of expression level differences between samples may be set to evaluate change in exon RNA abundances only above the change observed by RNA expression. Likewise, filters of exon RNA abundance between 41 breast cancer cell lines may be set to vary at up to 8-fold variation. Optionally, the filter of exon RNA abundances may be set to vary at up to 3-fold variation, or at up to 2-fold variation. Differential exon abundance levels is therefore metered both by exon RNA expression maximal changes between subgroups, and by the relative values that are observed and present above and beyond the potential RNA expression differences. For example, if the differential exon RNA abundance is set at <2-fold, all probe set variations for every gene must not exceed a 2-fold variation between the classifier subgroups in the high TI set versus the low TI set.

In this example, exon microarray data collected from breast cancer cell lines were analyzed using the FIRMA algorithm (as implemented by AltAnalyze) to determine which exons were differentially expressed. The FIRMA algorithm takes a set of raw microarray data (CEL files), splits into two classes, and determines which exons are most differentially expressed at a statistically significant level between the two classes. The AltAnalyze ouput files contain information on the degree of expression difference (fold-change) and several statistical measurements of the significance of the expression difference. In addition, for each exon, a measurement of the differential expression of the gene containing that exon is also provided. Exons were disregarded in subsequent analysis if the probeset p-value (a measurement of the confidence of the underlying exon expression measurement) was greater than 0.05. Exons for which the FIRMA p-value (a measurement of the exon differential expression) was greater than 0.05 were also disregarded. Finally, exons for which the differential expression of the gene containing the exon was greater than three-fold were also disregarded. The threshold for this final filtering step is arbitrary, and its main purpose is to remove exons for which the simple measurement of the overall gene expression level would be just as effective as the more difficult measurement of the exon expression difference. Therefore, the thresholds may be modulated in different ways to influence the list size of exon probesets outputted.

In the method here, the FIRMA algorithm was conducted by requiring that the input data be separated into two classes, such that exons that are differentially expressed at a statistically significant level are determined between these two classes. For the tumor initiating (TI) score classification, the TI gene set score was computed for each cell line, and a subset of the cell lines were classified as TI-high (having an TI score greater than zero) or TI-low (the lowest-scoring cell lines). The cell lines used for the TI gene set score classification were determined. A tumor-initiating (TI) score (based on a tumor-initiating gene set signature) was computed for each cell line, and a subset of the cell lines was classified as TI-high (having a TI score greater than zero) or TI-low (the lowest-scoring cell lines). Cell lines in each class were:
a. TI-high: SUM149, BT_{—}549, MDA-MB-436, MDA-MB-157, CAL-120, SUM1315, SUM159, Hs578T, HCC1395, MDA_MB_{—}231, HCC1806
b. TI-low: ZR75-30, HCC1419, T47D, SUM52, HCC1428, BT483, ZR75-1, HCC1500, MDA-MB-361

In the example illustrated in FIG. 5, the tumor initiating gene set score was used as a discriminator to identify two groups of cell lines with TI (high) and TI (low) gene classifications. Upon execution of the method, gene isoforms represented by alternatively spliced exons that are measured by exon-specific probesets are evaluated, and a range of outputs is developed that have maximal to minimal differences in abundances for every probe set. An alternative splicing predictor is implemented (FIRMA, splicing index and MiDAS algorithms). The derivation of differential values for every probeset for the transcriptome is assessed for statistical relevance by p-value and multiple-testing adjusted algorithm p-values. By comparing these two groups, a total of 932 exon probesets were ranked as differential exons based on a >2-fold change in the normalized probeset expression value. FIG. 5 illustrates the pattern of expression amongst the 41 breast cancer cell lines, and it is evident that the pattern is displayed into two main types. Exon probesets were clustered for pattern similarity. First, approximately half of the exon probesets were demonstrated to have a TI(high)-included, TI(low)-deleted pattern. Second, the other half of the exon probesets were shown to have the opposite TI(high)-deleted, TI(low)-included pattern. Exon probesets are identified in Table 1 and Table 2.

In another example of the method, exon abundance variations may be set at up to 8-fold (<8-fold) variation, or optionally may be set at up to 3-fold (<3-fold) variation. The visualization of the expression pattern of these probesets amongst all the samples (41 breast cancer cell lines) illustrates that the group of probesets define cells with a tumor initiating signature, composed of classes of alternatively spliced exons that are included and others that are excluded in these cells. A tabulation of the complete TI probesets is presented in Table 1 and Table 2. Thus, both Gene isoforms that are increased in expression and others that are reduced in expression may contribute to defining cells with the tumor initiating features.

An unsupervised hierarchical clustering is useful to establish the relationship between samples in the group in an unbiased manner. In another TI classifier exercise, N=577 exon probesets exhibiting <8-fold variation were evaluated to determine the relatedness of the 41 breast cancer cell lines. The TI classifier identifies a high TI, high EMT, and basal-B like cell line subgroup [Group 1] composed of BT549, SUM1315, MDA.MB.231, Hs578T, SUM159, MDA.MB.157, MDA.MB.435, MDA.MB.436, SKBR.7, that was observed to be statistically significantly different from the other breast cancer cell lines with AU (100)/BP (99). Also, within the luminal type cell lines, the TI classifier was observed to statistically significantly distinguish additional breast cancer cell lines into two subgroups with AU (83)/BP(14) in the cluster dendogram. The two Luminal subgroups distinguished were [Group 2, SUM44, MCF7, T47D, MDA.MB.175VIII, SUM185, BT474, MDA.MB.361, MDA.MB.330, UACC812, ZR75.1, BT483, CAMA.1] and [Group 3, MDA.MB.415, MDA.MB.468, MPE600, SUM52, ZR75.30, SUM190, SUM225, UACC893, SK.BR.3, SK.BR.5, EVSA.T, OCUB.M]. Thus, the cluster dendograms reveal similarity between cell lines assigned by the exon probesets from the TI discriminator. The assignments may be conducted to identify similar groups of tumor samples.

### Example 4

### Basal-B Discriminator for Breast Cancer Classification

The basal-B subtype of breast cancers (BaB) are a particularly aggressive form of breast cancer. Although certain basal-like cancers are treatable with standard chemotherapy, a higher fraction of these cancers are resistant to chemotherapy, and no adequate treatment options are available. Basal-like breast cancers may be identified by signatures of differential splicing where change in the pattern of splicing is indicative of a biological process relevant to the cancer progression, maintenance, differentiation, de-differentiation, transition, interaction with other cell types, metastasis, micro-colonization, tumor dormancy, tumor growth, and the like. A pattern or classifier may be established by discovery of exons from the same gene, or by exons of different genes with a similar pattern, whereas exons elsewhere in the same gene and in different genes may adhere to an alternatively patterned classifier. Although a single classifier portraying an alternatively spliced exon of one type is valuable, additional information may be gained by analysis of multiple types.

In the method, a group of samples is evaluated for whole transcriptome profiling using measurements of exons via probesets on microarray chips, Q-PCR, and or RNA sequencing strategies. Under these circumstances abundances of each exon are determined. The samples are ordered by a classification schema. In this case, the classification is implemented by determination of a subgroup of samples with basal-B characteristics based on gene expression, molecular and protein markers, and cell morphology. Similarly, distinct groups of cells that are luminal by morphology, gene expression, molecular and protein marker distributions of also defined as an opposing classifier subgroup for distinguishing exon probesets governed by the filtering criteria.

In the method, the expression level of genes (RNA expression) may be compiled and used to filter the output of alternatively spliced exons (gene isoforms). In this regard, filters of expression level differences between samples may be set to evaluate change to exon RNA abundances only above the change observed by RNA expression. Likewise, filters of exon RNA abundance between 41 breast cancer cell lines may be set to vary at up to 8-fold variation. Optionally, the filter of exon RNA abundances may be set to vary at up to 3-fold variation, or at up to 2-fold variation. Differential exon abundance levels is therefore metered both by exon RNA expression maximal changes between subgroups, and by the relative values that are observed are present above and beyond the potential RNA expression differences. For example, if the differential exon RNA abundance is set at <2-fold, all probe set variations for every gene must not exceed a 2-fold variation between the classifier subgroups in the basal-B subtype set versus the non-basal-B set (eg. luminal, luminal-A, basal-A, or normal-like).

Exon microarray data collected from the cell lines listed above were analyzed using the FIRMA algorithm (as implemented by AltAnalyze) to determine which exons were differentially expressed. The FIRMA algorithm takes a set of raw microarray data (CEL files), splits into two classes, and determines which exons are most differentially expressed at a statistically significant level between the two classes. The AltAnalyze ouput files contain information on the degree of expression difference (fold-change) and several statistical measurements of the significance of the expression difference. In addition, for each exon, a measurement of the differential expression of the gene containing that exon is also provided. Exons were disregarded in subsequent analysis if the probeset p-value (a measurement of the confidence of the underlying exon expression measurement) was greater than 0.05. Exons for which the FIRMA p-value (a measurement of the exon differential expression) was greater than 0.05 were also disregarded. Finally, exons for which the differential expression of the gene containing the exon was greater than three-fold were also disregarded. The threshold for this final filtering step is arbitrary, and its main purpose is to remove exons for which the simple measurement of the overall gene expression level would be just as effective as the more difficult measurement of the exon expression difference. Therefore, the thresholds may be modulated in different ways to influence the list size of exon probesets outputted.

In the method here, the FIRMA algorithm was conducted by requiring that the input data be separated into two classes, such that exons that are differentially expressed at a statistically significant level are determined between these two classes. For the tumor initiating (TI) score classification, the TI gene set score was computed for each cell line, and a subset of the cell lines were classified as TI-high (having an TI score greater than zero) or TI-low (the lowest-scoring cell lines). The cell lines used for the TI Gene set score classification were determined. A tumor-initiating (TI) score (based on a tumor-initiating gene set signature) was computed for each cell line, and a subset of the cell lines was classified as TI-high (having a TI score greater than zero) or TI-low (the lowest-scoring cell lines). Cell lines in each class were categorized as BasalB vs Luminal based on histopathology evaluations from the original tumors, and annotated with a “type”, classifying them as basal-A, basal-B, luminal, or unknown. Seven cell lines annotated as either basal-B or luminal for this classification were selected:
a. Basal-B: SUM149, BT_{—}549, MDA-MB-436, MDA-MB-157, SUM159, Hs578T, MDA_MB_{—}231
b. Luminal. MCF7, MDA-MB-453, SK-BR-3, BT474, T47D, ZR75-1, MDA-MB-361

Upon execution of the method, gene isoforms represented by alternatively spliced exons that are measured by exon-specific probesets are evaluated, and a range of outputs is developed that have maximal to minimal differences in abundances for every probe set. An alternative splicing predictor is implemented (FIRMA, splicing index and MiDAS algorithms). In the example, 41 human breast cancer cell lines were rank ordered following outputting of probesets from the transcriptome microarray. High values in the spectrum coinciding with cell lines in the group having basal-B cell type positivity, and low values in the scoring associated with other cell lines having luminal cell type positivity, The derivation of differential values for every probeset for the transcriptome is assessed for statistical relevance by p-value and multiple-testing adjusted algorithm p-values. There are N=320 probesets found at a p<0.05 accounting for multiple sampling. Also, exon abundance variations may be set at up to 8-fold (<8-fold) variation, or optionally may be set at up to 3-fold (<3-fold) variation. The visualization of the expression pattern of these probesets amongst all the samples (41 breast cancer cell lines) illustrates that the group of probesets define cells with a basal-B signature, composed of classes of alternatively spliced exons that are Included and others that are Excluded in these cells. A tabulation of the complete BaB probesets is presented in Table 1 and Table 2. Thus, both Gene isoforms that are gained and others that are lost may contribute to defining cells with the BaB features.

### Example 5

### Concordant Exon Signature

Cancer stem cells are likely to possess features of tumor initiating cells and have some attributes determined by an epithelial-to-mesenchymal transition (EMT). For breast cancer, basal-like morphology may also be connected to cancer stem cells. Importantly, each discriminator leads to the identification of a related subgroup of the breast cancers indicating that they may each be probing different attributes of the same tumor cell biology. Importantly, the combination of these features rather than the application of only one of the three features, may add additional insight into an ability to stratify patients and identify exon biomarkers that are meaningful for therapy responsiveness.

To evaluate combined influences of exons discovered from three of the discriminators: tumor initiating (TI), EMT, and basal B-like, the concordance of these groups was evaluated. The concordance between TI, basal-B, and EMT exon lists (Table 1 and Table 2) indicates the representation of certain exons and gene isoforms in all three lists (133 Exon probesets contributing to N=40 genes) (FIG. 6). Notably, the concordant group of exons are identifying and assigning a significant group of breast cancer specimens that are high for tumor initiating, EMT, and basal-B type based on the output similarity from unsupervised hierarchical clustering. Further, it is demonstrated that the exons were in two groups consistent with the differential expression discriminator: those that have increased expression of the exons in high tumor initiating, mesenchymal-type, and basal B-type represented approximately two-thirds of the total group, and are listed in Table 1.

Likewise, an another group of exons were underexpressed in high tumor initiating, mesenchymal-type, and basal B-type are listed in Table 2.

In addition to the concordance amongst all three groups, there is significant overlap between tumor initiating and basal-B exon subgroups (N=353), between tumor initiating and EMT exon subgroups (N=70), and between EMT and basal-B Exon subgroups (N=48) (FIG. 6). In evaluating particular exon probesets, it is interesting that there are two probeset groups for TGFB1I1 [3657205 and 3657205], KIAA1543 [3818976 and 3818987], ARRDC1 [3195364 and 3195386] and ATP2C2 [3671792 and 3671770] of the high tumor initiating, EMT, and basal-B type. Also, LIMA1 [3454368 and 3454365] has two probesets of the low tumor initiating, EMT, and non-basal-B type. Notably, the gene ENAH and the probeset of the 11a ENAH isoform is exhibited to have the low Tumor Initiating, low EMT (Epithelial-like), and non-basal-B type pattern. Exons from this group are listed in the Table 1 and Table 2.

### Example 6

### Identification of Exon Differential Expression Patterns in Mesenchymal-Like Cells, Epithelial-Like Cells, and Fibroblasts

Tumors are composed of multiple different cell types including cells of non-tumor origin. It is important to distinguish the properties of the different cell types regarding cancer progression and therapy responsiveness. In the case of cancer stem cells and the epithelial-mesenchymal transition, it is clear that tumor heterogeneity is significant in the biological transitions and cell niches that are features of specialized tumor cell environments. Non-tumor cells, such as myofibroblasts, fibroblasts, stromal, and inflammatory cells may be present in tumor specimens, and may contribute to general gene expression measurements if not considered separately. These other cell types are also reflective of different properties of tumors including angiogenesis, inflammation, and hypoxia. Thus, it is desirable to identify biomarkers, and/or specifically selected genes and exons that may be expressed to different extents in these compartments. Also, it is desirable to identify tumor-specific biomarkers that are not found in the non-tumor cell types.

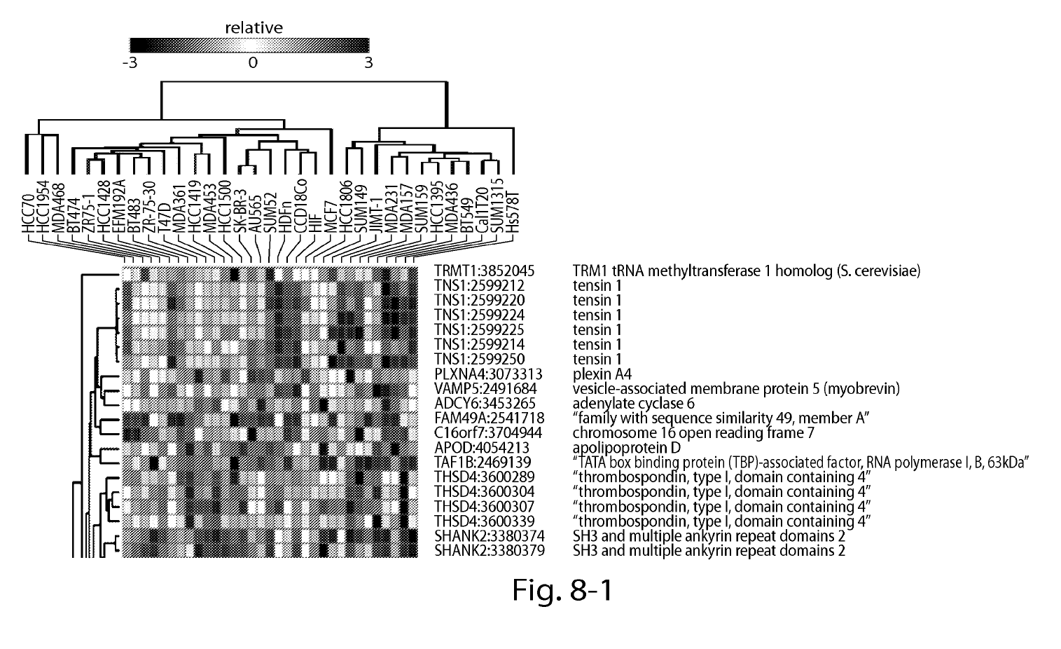

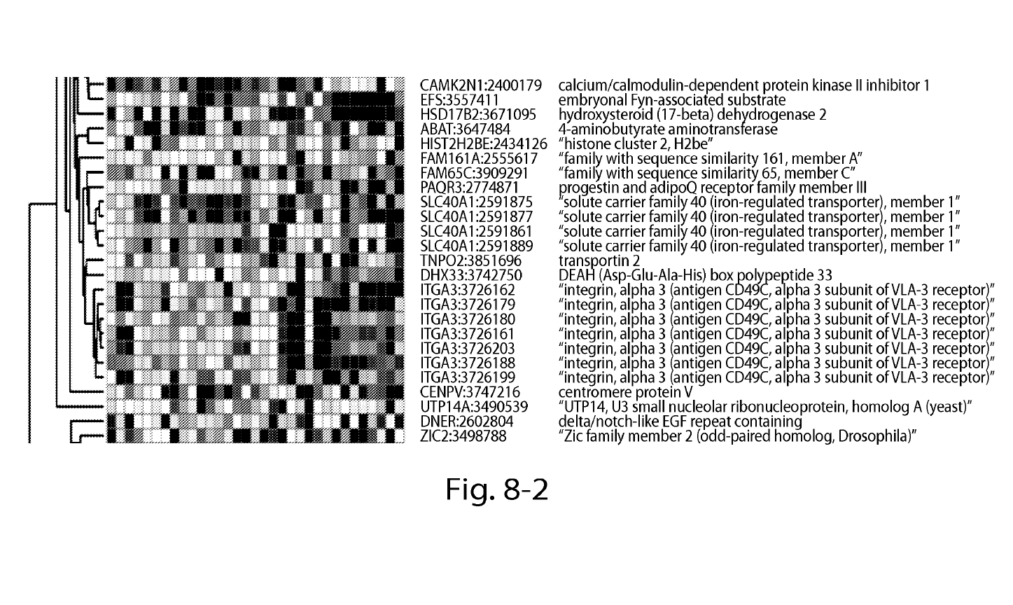

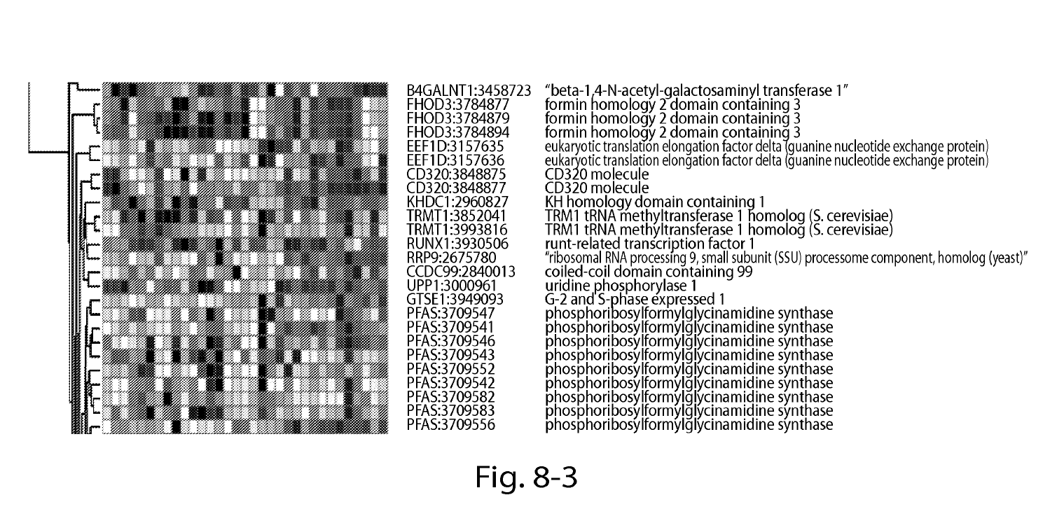

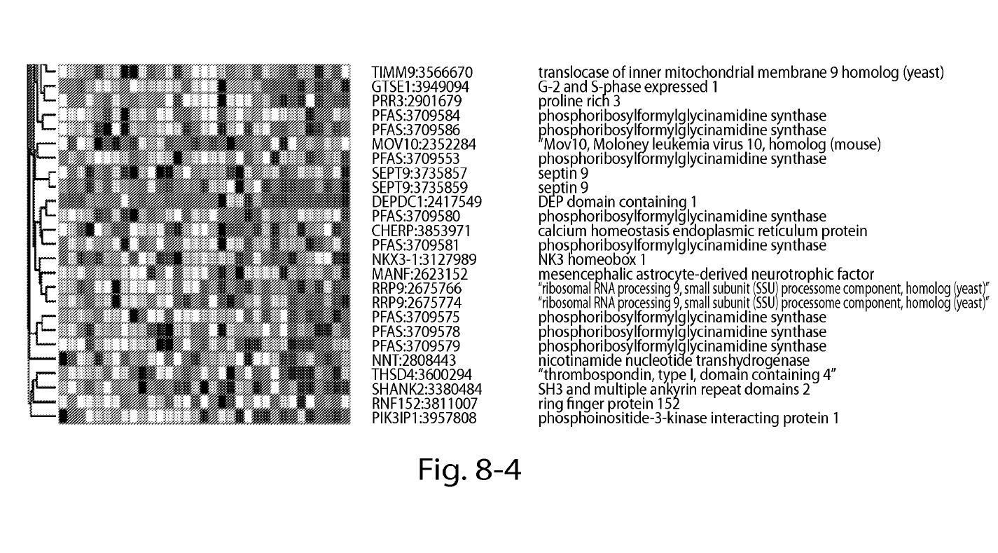

In this example, the exon discovery process was utilized to discriminate exon probesets that were present in a tumor, but absent or at reduced levels in a selected group of relevant non-tumor cells. A discriminator for this process consists of two components. First, exon lists are formed by the discriminator between mesenchymal-like and epithelial-like differential expression. Second, exon lists are filtered for exon probesets that are present in one of these two conditions, but also absent or reduced in fibroblasts. For the discovery process, the human fibroblast cell lines were HDFn, CCD18Co, and HIF, consisting of two fibroblast and one myofibroblast cell line. As is shown in FIG. 8, a group of 108 differentially expressed exon probesets were delineated. Additionally, 61 Exon probesets were M (mesenchymal)-included, E (epithelial)-deleted, and Fibroblast-deleted (Table 3). Of these, 16 exon probesets were identified from the PFAS gene, and no PFAS exon probesets were observed in the enriched M-deleted, E-included, and Fibroblast-included subgroup. Additionally, 47 exon probesets were M-deleted, E-included, and Fibroblast-included (Table 4). Of these, the alpha3 integrin, ITGA3, was represented with 7 exon probesets. As an indicator of differential splicing between cells of different types, it was found that the SHANK2 gene had a mixture of exon probesets that were either present in the M-deleted, E-included, and Fibroblast-included [2 exon probesets] or the M-included, E-deleted, and Fibroblast-deleted [1 exon probeset] groups. Exon probes may be evaluated using in situ hybridization technologies to identify the cells in a specimen where the exon is expressed. The pattern of exon expression would be informative about the preponderance of mesenchymal-like tumor cells distinct from fibroblasts in a complex specimen. The identification of exons that are differentially expressed between cell types is a valuable step towards using the exon biomarkers singly, or in combination, or in an exon signature, to define attributes of tumors as an indicator of patient stratification and therapy responsiveness. An exon signature containing specific exon biomarkers that are indicators of specialized cell types is valuable to use in complex tumor specimens where total gene isoform determinations are derived from unfractionated samples. Exons from this group are listed in the Table 3 and Table 4.

### Example 7

### Differential Exon Expression in Breast Cancer Subtypes

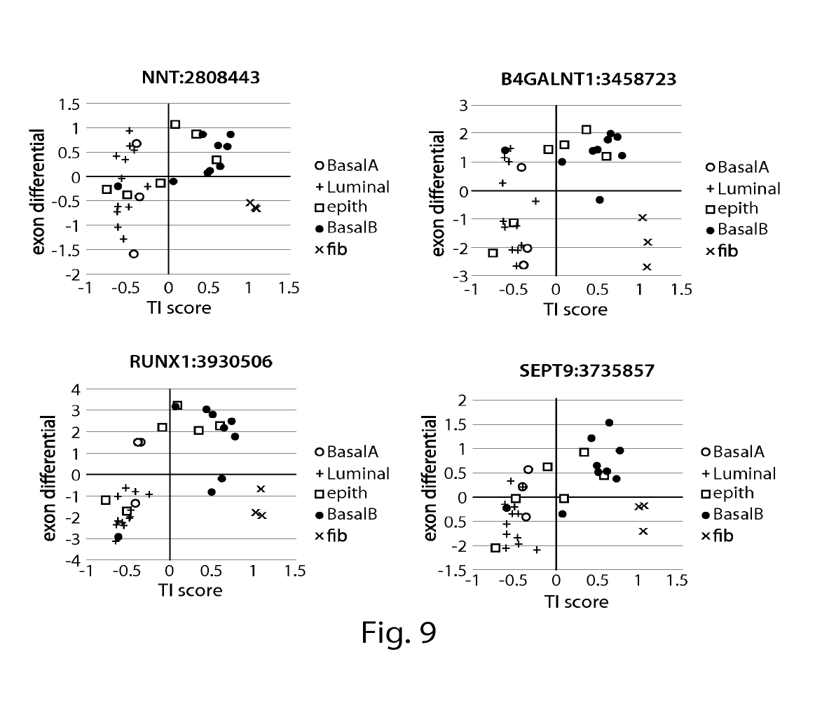

An exon that is differentially expressed between samples may be a useful biomarker for the presence of a cell type. Single exons, to the extent that the signal from the exon is discriminatory, are also valuable because fewer biomarkers may be easier implemented in clinical diagnostic settings. In this example, selected exon probesets identified from the tumor initiating, EMT, and basal-B discriminator methods were evaluated for the pattern of expression amongst breast cancer cell lines of differing subtypes. As shown in FIG. 9, basal-A, luminal, epithelial, basal-B breast cancer subtypes and fibroblast cell lines were compared for whether a single exon probe [4 shown] adequately separates basal-B cell lines from other breast cancer subtypes and other cell lines, when reflected relative to the rank tumor initiating score. Four different exon probesets were evaluated (NNT:2808443, B4GALNT1:3458723, RUNX1:3930506, and SEPT9:3735857). Certain basal-B and epithelial breast cancer cell lines were not distinguished by a single exon probeset evaluation. The overall conclusion from this analysis is that combinations of TI score signatures with any of these four exons will identify a large fraction of the basal-B cell lines separately from other cell types and fibroblasts. Algorithms derived from the exon probeset and TI gene signatures will be informative.

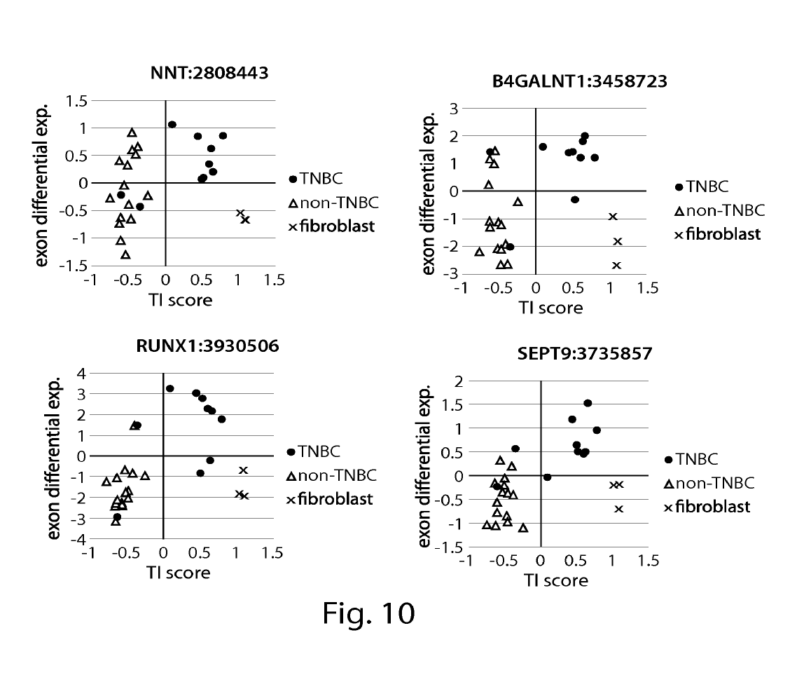

In another example, selected exon probesets identified from the tumor initiating, EMT, and basal-B discriminator methods were evaluated for the pattern of expression amongst breast cancer cell lines that were triple negative breast cancer, or other breast cancer subtypes that were not triple negative breast cancer. As shown in FIG. 10, triple negative breast cancer cell lines were primarily distinguished from non-triple negative breast cancer cell lines by using the expression values plotted for each exon. Likewise, most triple negative breast cancer cell lines were distinguished from fibroblasts with each exon. Four different exon probesets were evaluated (NNT:2808443, B4GALNT1:3458723, RUNX1:3930506, and SEPT9:3735857). Certain triple negative breast cancer cell lines were not distinguished by a single exon probeset evaluation. The overall conclusion from this analysis is that combinations of TI score signatures with any of these four exons will identify a large fraction of the triple negative breast cancer cell lines separately from non-triple negative breast cancer cell lines and fibroblasts. Algorithms derived from the exon probeset and triple negative gene signature classifiers will be informative.

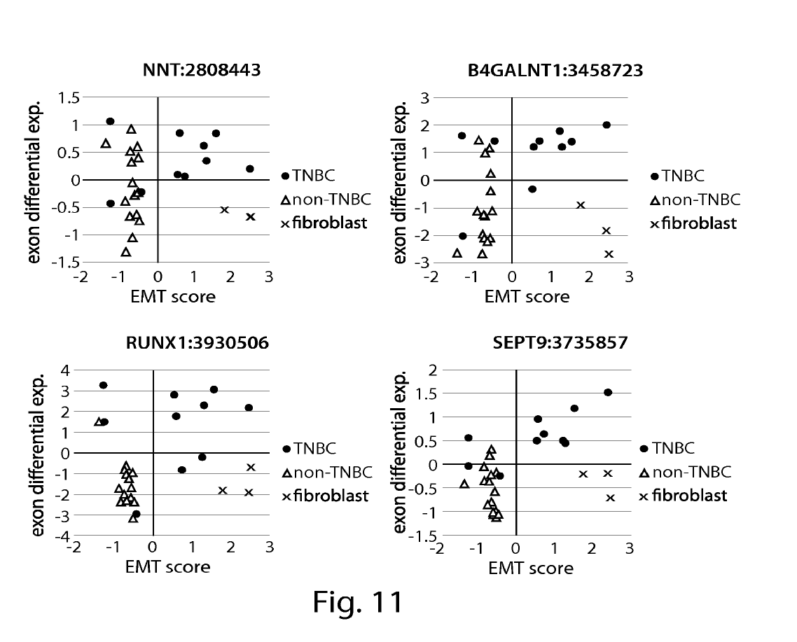

In another example, selected Exon probesets identified from the tumor initiating, EMT, and basal-B discriminator methods were evaluated for the pattern of expression amongst breast cancer cell lines that were triple negative breast cancer, or other breast cancer subtypes that were not triple negative breast cancer. As shown in FIG. 11, triple negative breast cancer cell lines were primarily distinguished from non-triple negative breast cancer cell lines by using the expression values plotted for each exon relative to the EMT gene score. Likewise, most triple negative breast cancer cell lines were distinguished from fibroblasts with each exon. Four different exon probesets were evaluated (NNT:2808443, B4GALNT1:3458723, RUNX1:3930506, and SEPT9:3735857). Certain triple negative breast cancer cell lines were not distinguished by a single exon probeset evaluation. The overall conclusion from this analysis is that combinations of EMT score signatures with any of these four exons will identify a large fraction of the triple negative breast cancer cell lines separately from non-triple negative breast cancer cell lines and fibroblasts. Algorithms derived from the exon probeset and EMT gene signature classifiers will be informative for identifying these cancers.

### Example 8

### Tumor Initiating Gene Score and Differential Exon Discovery

Three discriminators are defined for the splicing index process algorithm. These are two-way discriminators for tumor initiating (TI), non-tumor initiating (nonTI), EMT(high)-EMT(low), and basal-B luminal [a morphology determinant]. The cut-off criteria imposed was at a p<0.001 having >2-fold exon change but restricted by <3 fold gene expression change. Operationally, 3 T tests are formed for positive TI versus negative TI, positive EMT versus negative EMT, and basal-B versus luminal. In this exercise, the TI discriminator yielded 134 exon probesets within the cutoff criteria. The EMT discriminator yielded 135 probesets within the cutoff criteria. The basal-B versus luminal discriminator yielded 132 probesets within the cutoff criteria. The sum of pairwise combinations of the three tests yields the union group; the intersection of three tests yields the concordant group. Exons from this group are listed in the Table 5 and 6.

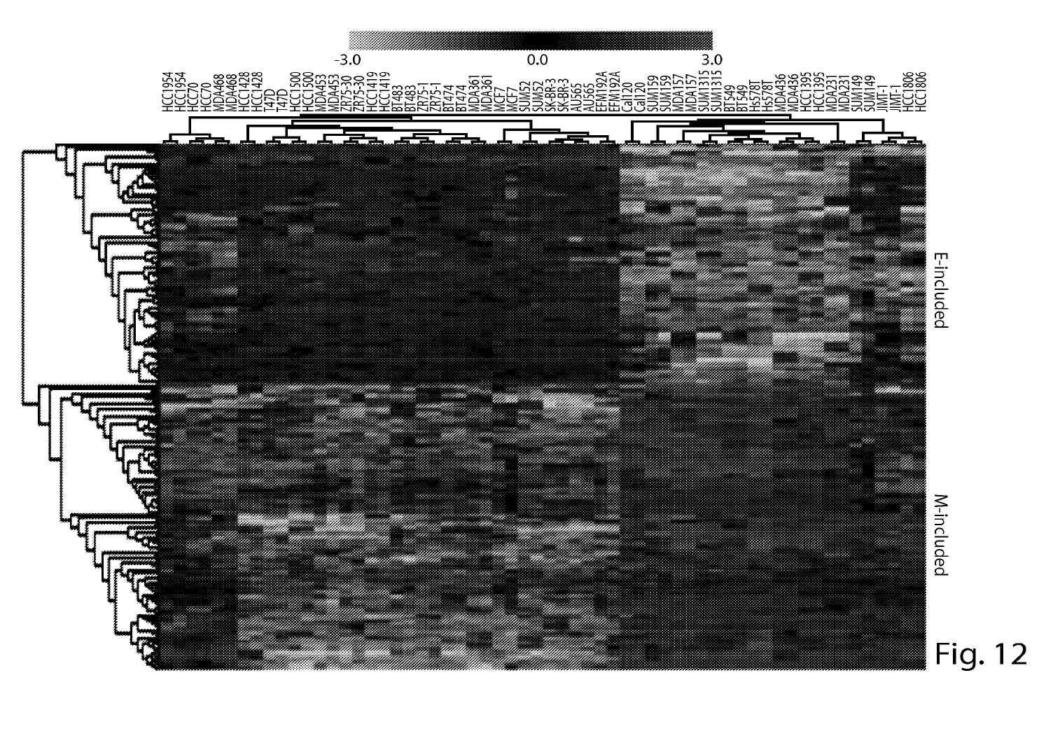

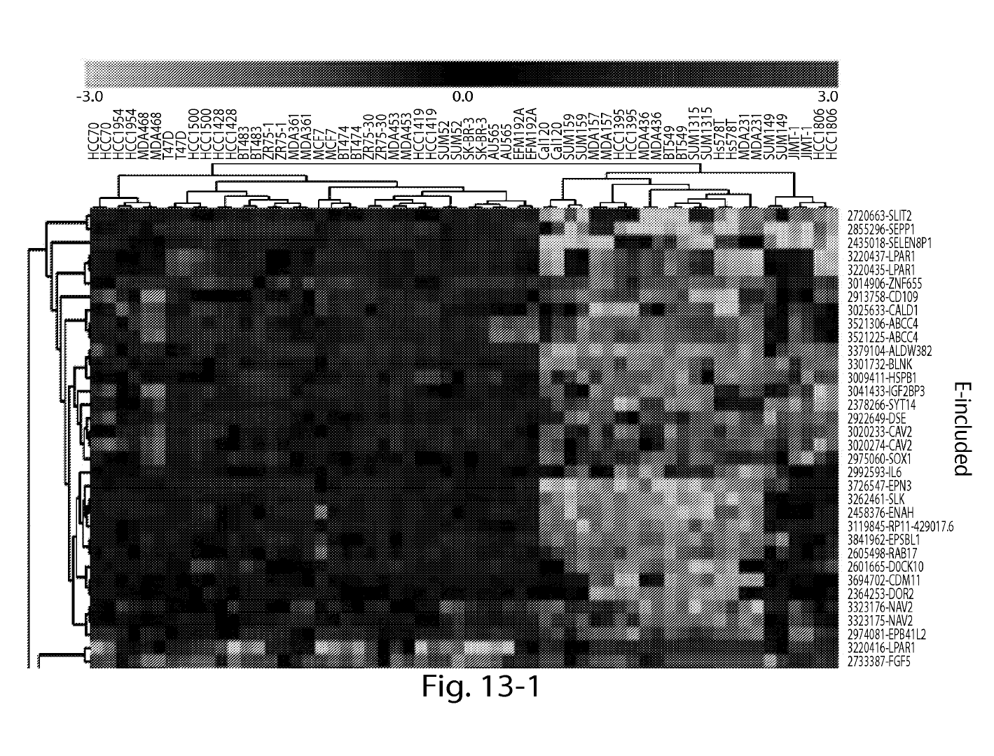

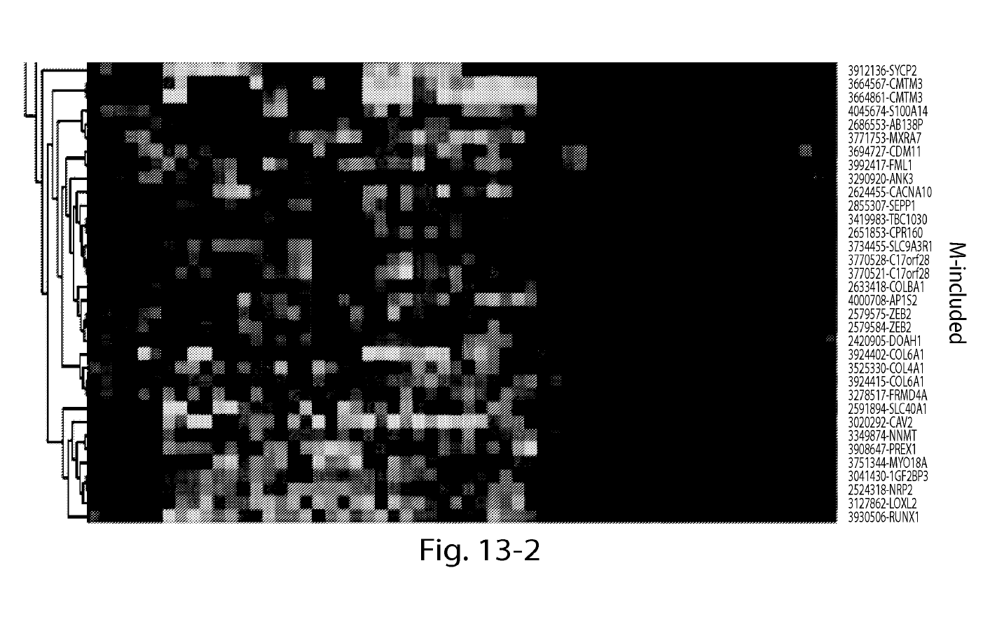

A hierarchical clustering based on the concordance or union of three sets [discriminators for tumor initiating (TI), non-tumor initiating (nonTI), EMT(high)-EMT(low), and basal-B luminal [a morphology determinant]] was conducted. The output from this analysis was displayed as unsupervised clustering of human breast cancer cell lines versus similarity of individual Exon probesets (FIG. 12 and FIG. 13). As shown in the FIG. 12, the union group of probesets sort breast cancer cell lines into defined groups. Likewise, the union group of probesets are separated into two primary subsets: E-included (exon probesets indicative of exons with high relative expression in TI(low), EMT (low), non-basal B, or epithelial breast cancer cells] and M-included (exon probesets indicative of exons with high relative expression in TI(high), EMT(high), basal-B or mesenchymal-like breast cancer cells). As evidenced in FIG. 12, approximately one-half of the exon probesets reveal differential expression of each of the two primary subsets.

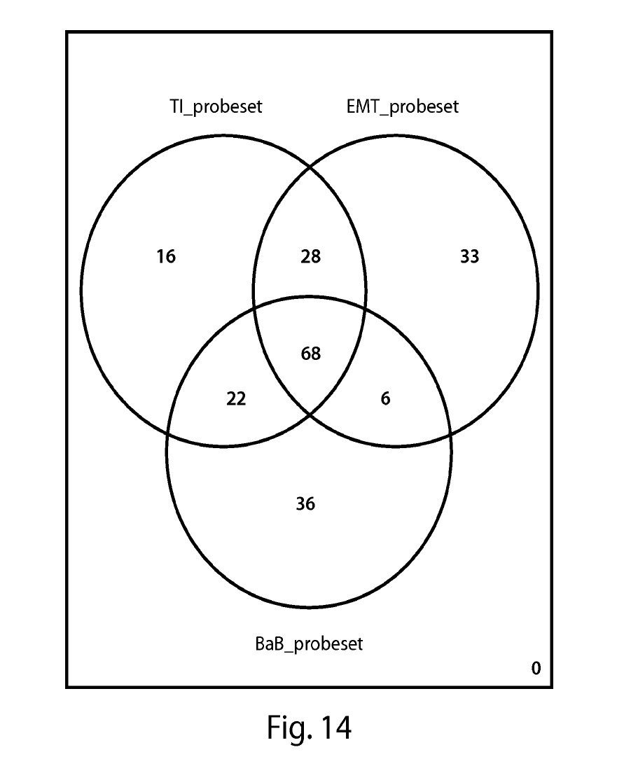

As shown in the FIG. 13, the concordant group of probesets are observed to sort breast cancer cell lines into defined groups. Likewise, the union group of probesets are separated into two primary subsets: E-included (Exon probesets indicative of exons with high relative expression in TI(low), EMT (low), non-basal B, or epithelial breast cancer cells) and M-included (exon probesets indicative of exons with high relative expression in TI(high), EMT(high), basal-B or mesenchymal-like breast cancer cells). It is found that 23 genes are represented in the 68 exons, where 36 of the exons are upregulated in the TI(high), EMT(high), basal-B or mesenchymal-like breast cancer cells (Table 5). A Venn diagram illustrates the degree of overlap from the intersection of the three pairwise discriminators used in the analysis (FIG. 14). A level of high significance was observed with a T test calculation to p=6.3e-6.

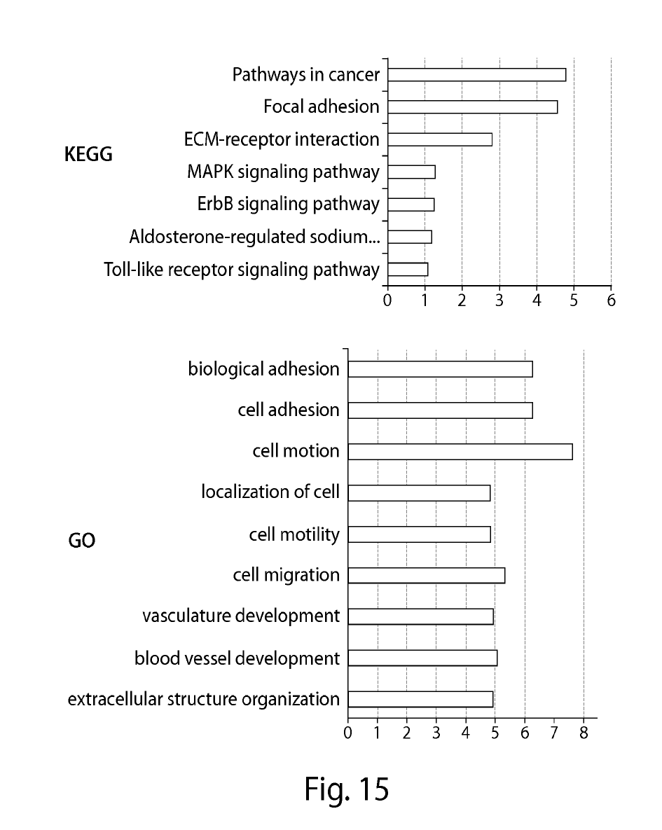

The exon probesets derived from splicing index algorithms from the union[209 exons] of three discriminators [tumor initiating (TI), non-tumor Initiating (nonTI), EMT(high)-EMT(low), and basal-B luminal] are analyzed in for biological pathway connectivity using KEGG and GO software. As shown in FIG. 15, KEGG output showed high log 10(P) significance for pathways in cancer log 10(4.77), focal adhesion log 10(4.56), ECM-receptor interaction log 10(2.81). Benjamini-Hochberg false discovery rates (q) were computed to be <0.1 for these terms. A trend was observed for MAPK signaling pathway and ErbB signaling pathway also, aldosterone-regulated sodium reabsorption and Toll-like receptor signaling pathway. In addition for GO biological network the following terms are presented with high significance, biological adhesion (5.31e-07), cell adhesion (5.19e-07), cell motion (2.31e-08), localization of cell (1.37e-05), cell motility (1.37e-05), cell migration (4.68e-06), vascular development (1.1e-05), blood vessel development (8.79e-06), and extracellular structure organization (1.17e-05). Benjamini-Hochberg false discovery rates (q) were computed to be <0.1 for these terms.

An important feature of this discovery is the finding that exons delineated from the FIRMA and splicing index algorithms are distinctive exon sets with very low concordance with the tumor initiating and/or EMT gene signatures. As such, the identified differentially expressed exons are generated by a novel strategy, and are valuable biomarkers correlating with the cancer stem cell/tumor initiating/EMT patterns of tumor cell properties in tumors.

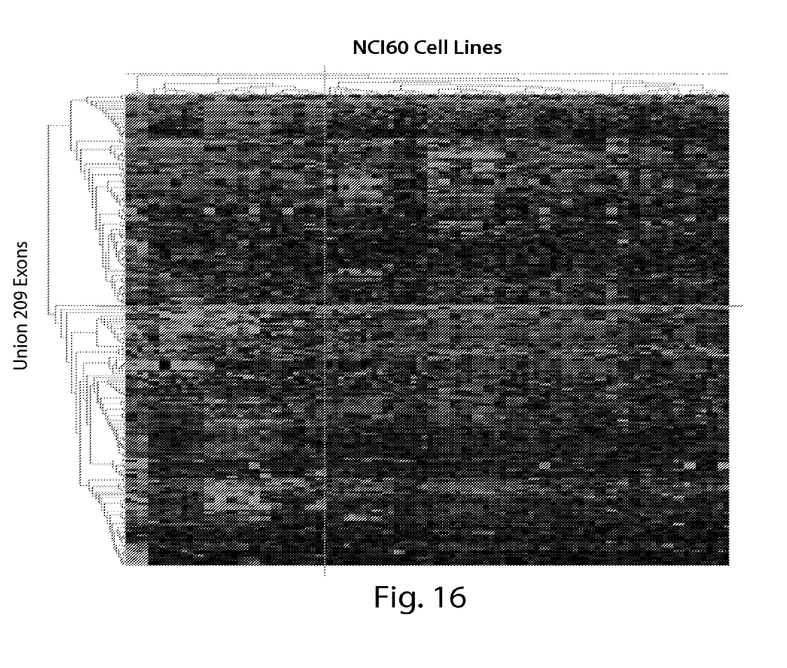

To test the predictive capacity of the exon signatures of TI/EMT/BaB from splicing index (SI) or FIRMA with new cancer specimens, the exon signature was evaluated in a new sample set to determine whether the samples of differing exon expression pattern types may be discriminated. As shown in FIG. 16, an unsupervised hierarchical clustering with union (n=209) exon signature was observed to separate the tumor cell lines from the NCI60 panel into related subgroups. NCI60 cell lines are a collection of cancer type origin, including breast, lung, pancreatic, leukemia, colorectal, ovarian, and other types. Support vector machine analysis of the independent NCI-60 cancer cell line dataset determined that the top 60 exons from the breast cancer cell line training group identified 96% of the CSC-high cell lines and 90% of CSC-low cell lines with high accuracy. These observations indicate that the exon signature is able to distinguish cancer types based on TI/EMT/Ba selection criteria, and indicates that the cancer stem cell (CSC) characteristics may be found in other tumor types.

In the method, the centroid procedure was utilized to develop a discriminator for cell type evaluation based on gene and exon signatures. Centroids are used to gauge the distance of similarity. In this process, the method used is to build up two-way discriminator centroids based on exon array data. There is an average of the 2 clusters from training datasets, and the centroids are then normalized.

In one example of the centroid for tumor initiating signatures, the gene signature centroid was outputted. In second and following examples, Exon signatures were applied to centroid building. Evaluation of cancer stem cell centroid models were assessed in human primary breast cancer specimens where full genome exon microarray datasets [Affymetrics Exon1.0] were used. In this process, 81 human primary breast cancers were acceptable for comparison. In this group, there is a representation of HER2 positive, luminal and basal breast cancers based on histopathology and morphological criteria from pathology review. In order to compare the centroid output with identifiable gene expression relevant to the breast cancer subtype, the same samples were also indexed for expression levels of three genes: ER, PR, and HER2. Visualization of centroids was displayed with unsupervised hierarchical clustering to illustrate relatedness. For both the CSC gene signature and the CSC exon signature, the centroids were built around a two group distinction called TI versus nonTI.

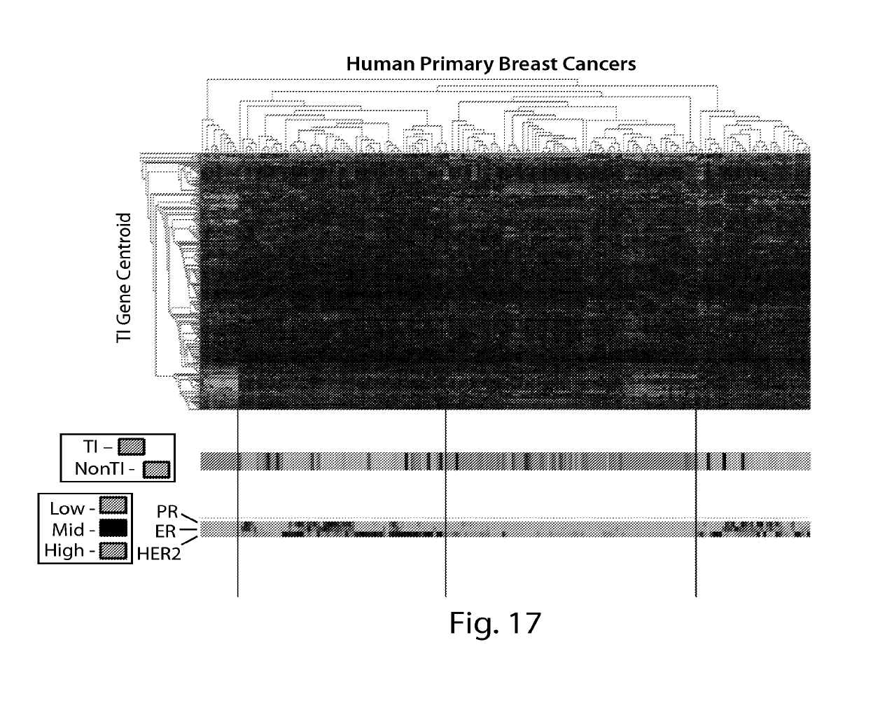

In the example of the CSC gene signature centroid applied to the human breast cancer specimens (FIG. 17, top panel), it was observed that the process grouped human breast cancers into distinct types with a hierarchical clustering display. To condense the information, a centroid rank distance was established to display similarity between any one human breast cancer specimen and the designation of either the TI or the non-TI group (FIG. 17 middle panel). As shown in FIG. 17, specimens associate best with either a TI or non-TI group in the centroid model. To determine the types of human breast cancer for which the TI group associates, a plot of ER, PR, and Her2 gene expression was displayed (FIG. 17, lower panel). It is observed that primary breast cancers that score High in the TI index are low for ER, PR, and Her2 expression generally.

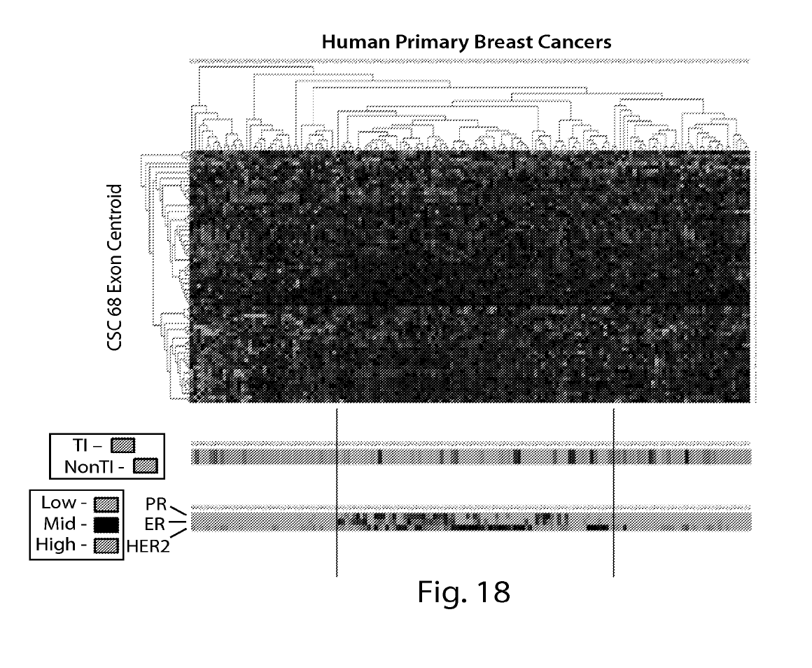

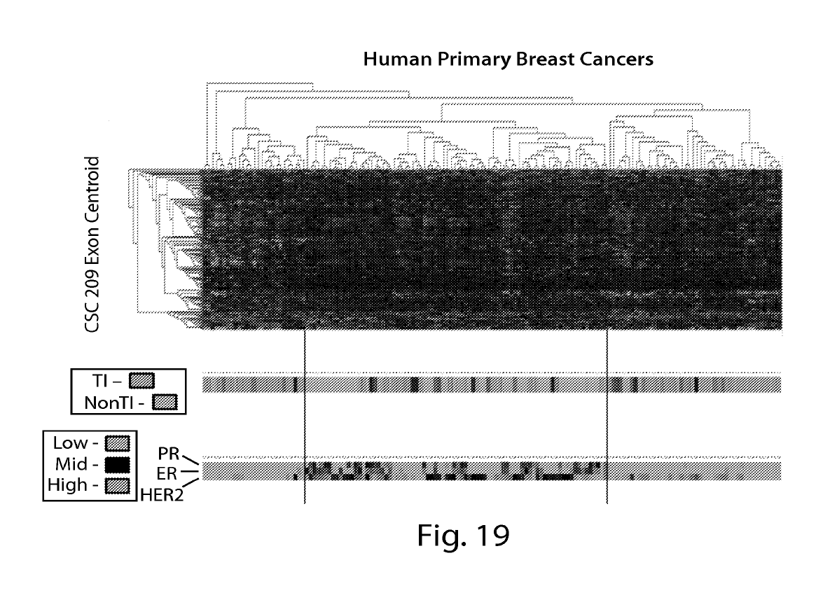

In the example of the CSC 68 Exon Signature centroid applied to the human breast cancer specimens (FIG. 18, top panel), it was observed that the process grouped human breast cancers into distinct types with a hierarchical clustering display. To condense the information as above, a centroid rank distance was established to display similarity between any one human breast cancer specimen and the designation of either the TI or the non-TI group (FIG. 18 middle panel). Likewise, the CSC 209 Exon Signature centroid applied to the human breast cancer specimens (FIG. 19, top panel), it was observed that the process also grouped human breast cancers into distinct types with a hierarchical clustering display. To further condense this information, a centroid rank distance was established to display similarity between any one human breast cancer specimen and the designation of either the TI or the non-TI group (FIG. 19 middle panel). As shown in FIG. 18 and FIG. 19, specimens associate best with either a TI or non-TI group in the examples of either exon centroid model. To determine the types of human breast cancer for which the TI group associates from the Exon centroids, a plot of ER, PR, and Her2 gene expression was displayed (FIG. 18, lower panel; FIG. 19, lower panel). It is observed that primary breast cancers that score High in the TI index from either the CSC 68 Exon centroid or the CSC 209 Exon centroid, are low for ER, PR, and Her2 expression generally. These examples illustrate the ability of the Exon centroid models to delineate cancers into type discrimination.

Centroid:centroid comparisons are useful to determine if each of the models are independently identifying similar human breast cancers. In the analysis of the output, a process including Spearman correlations are formed and for each sample there is a calculation of two number values. Values range from −1 to 1. In this context, positive (+) values is an indicator of a positive correlation and negative (−) values are indications of negative correlation. A Cohen Kappa value is computed for the set of centroid values from a group of specimens in a centroid:centroid comparison where 1 [perfect correlation], >0.7-0.8 [excellent correlation], >0.6 [substantial correlation], >0.4 [very good correlation], >0.2 [fair correlation], and >0.1 [not so great correlation] apply in the evaluation.

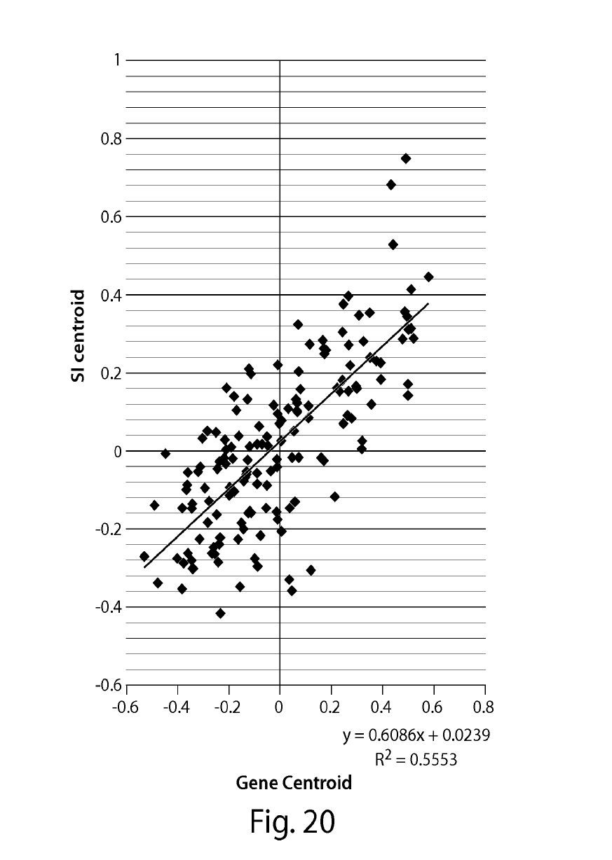

CSC exon signature and TI gene signature comparisons are illustrated in FIG. 20 for 81 human breast cancer datasets evaluated. Dots represent individual breast cancer specimen values for either centroid in the comparison. The data indicates a striking correspondence with an overall computed Cohen Kappa of 0.60 (substantial correlation).

An independent classifier for breast cancer may be used to evaluate the selection of breast cancer type, and this classifier may then be compared with the performance of centroid models. In one example, triple negative breast cancer classifiers are instructive (Lehman, 2011, J Clin Invest doi:10.1172/JCI45014; Rody, 2011; Breast Cancer Research 2011, 13:R97) because they are potentially more precise and inclusive than gene expression algorithms for only the three genes ER, PR, and Her2. The triple negative breast cancer (TNBC) classifier was formed and utilized with the 81 human primary breast cancer specimens.

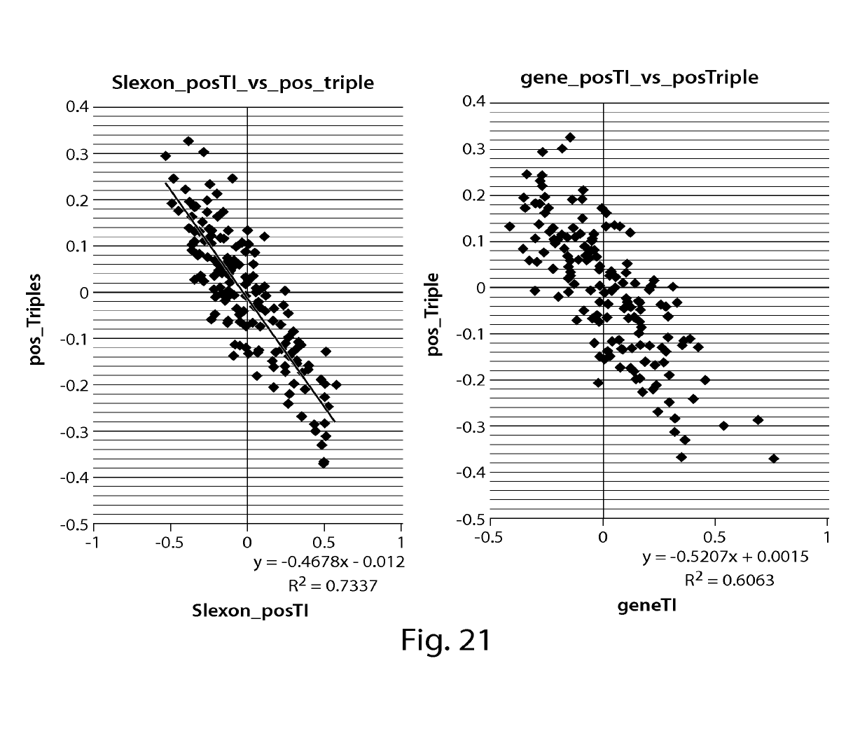

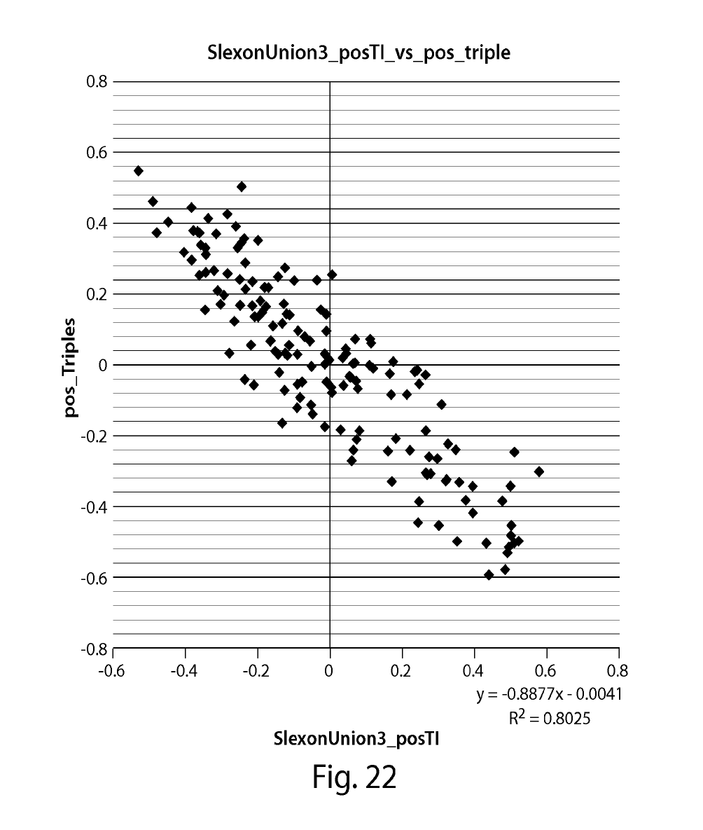

To determine the correlation between the CSC exon signature and the TI gene signature centroids with the TNBC classifier, multiple pairwise call comparisons were assembled to evaluate every human breast cancer specimen singly. The combined evaluation is displayed in FIG. 21. The left panel of FIG. 21 illustrates the strong correlation between TNBC (gene classifier) and the CSC 68 Exon centroid. The right panel of FIG. 21 illustrates the strong correlation between TNBC (gene classifier) and the TI gene centroid. Since these comparisons are between centroids and gene signatures, the degree of overall similarity is analyzed by R². For TNBC (gene classifier): CSC 68 Exon Centroid, the overall similarity has an R²=0.7337 (FIG. 21, left). For TNBC (gene classifier): TI Gene Centroid, the overall similarity has an R²=0.6063 (FIG. 21, right). In addition, the CSC 209 Exon Centroid demonstrated a strong correlation with the TNBC gene classifier with an overall similarity of R²=0.8025.

These methods identify key Exons representing gene isoforms that contribute to the identification of CSC, where the CSC description is formed from tumor initiating, EMT, and Basal B-like characteristics of breast cancer. The methods disclosed demonstrate the utility of exon biomarkers for characterization and typing of human breast cancers from general gene isoform expression values. These isoforms and the associated Exon identifiers [probesets] are valuable biomarkers for human cancer evaluation.

### EQUIVALENTS

Those skilled in the art will recognize, or be able to ascertain using no more than routine experimentation, many equivalents to the specific embodiments described herein. Such equivalents are intended to be encompassed by the following claims.

What is claimed:

1. A method of evaluating or treating a subject, comprising:
a) optionally, acquiring a subject sample;
b) acquiring a value or values that is a function of the level of expression of a plurality of gene isoforms from each of a plurality of gene isoforms from a first and/or second and/or third and/or fourth and/or fifth and/or sixth set of gene isoforms, wherein:
(i) said first set of gene isoforms comprises or consists of gene isoforms in Table 8; and
(ii) said second set of gene isoforms comprises or consists of gene isoforms in Table 9; and
(iii) said third set of gene isoforms comprises or consists of gene isoforms in Table 10; and
(iv) said fourth set of gene isoforms comprises or consists of gene isoforms in Table 11; and
(v) said fifth set of gene isoforms comprises or consists of gene isoforms in Table 12; and
(vi) said sixth set of gene isoforms comprises or consists of gene isoforms in Table 13; and
c) responsive to said value or values:
(i) classifying said subject (e.g., classifying said subject as a candidate for treatment with a preselected drug and/or treating, or withholding treatment from, said subject with a preselected drug); or
(ii) administering treatment comprising said agent that inhibits or kills cancer associated mesenchymal cells, tumor initiating cancer cells, or cancer stem cells to said subject; provided that, if (c)(ii) is not performed the acquisition in (a) or (b) comprises directly acquiring; thereby evaluating or treating said subject.

2.-11. (canceled)

12. The method of claim 1, wherein step b) said plurality comprises, or consists of, a first gene isoform.

13. The method of claim 1, wherein, in step b) said plurality comprises, or consists of, a first gene isoform and a second gene isoform.

14. The method of claim 13, wherein step b) comprises acquiring a value that is a function of the level of expression of a gene isoform of a first gene and the level of expression of a gene isoform of a second gene.

15. The method of claim 13, wherein step b) comprises acquiring a first value that is a function of the level of expression of said first gene isoform and a second value that is a function of the level of expression of said second gene isoform.

16.-26. (canceled)

27. The method of claim 1, wherein said value or values is a function of a comparison with a reference criterion.

28. (canceled)

29. The method of claim 1, comprising acquiring a values or values for the level expression of each of a plurality of gene isoforms of a gene.

30.-37. (canceled)

38. The method of claim 1, wherein said subject sample is a tumor sample.

39. The method of claim 1, wherein a first value or values is acquired for a first location in said subject sample.

40. The method of claim 39, wherein a second value or values is acquired for a second location in said subject sample.

41.-53. (canceled)

54. The method of claim 1, wherein said subject has cancer.

55.-83. (canceled)

84. A method of assaying in a subject sample the level of gene expression product of a plurality of genes selected from a first and/or second and/or third and/or fourth and/or fifth and/or sixth set of gene isoforms, wherein:
(i) said first set of gene isoforms comprises or consists of genes in Table 8,
(ii) said second set of gene isoforms comprises or consists of gene isoforms in Table 9; and
(iii) said third set of gene isoforms comprises or consists of gene isoforms in Table 10; and
(iv) said fourth set of gene isoforms comprises or consists of gene isoforms in Table 11; and
(v) said fifth set of gene isoforms comprises or consists of gene isoforms in Table 12; and
(vi) said sixth set of gene isoforms comprises or consists of gene isoforms in Table 13; comprising a first agent capable of interacting with a gene expression product of a plurality of genes selected from a first and/or second and/or third and/or fourth and/or fifth and/or sixth set of gene isoforms; and wherein the method comprises assaying the level of gene expression product of the plurality of gene isoforms.

85. The method of claim 84, comprising a second agent capable of interacting with a gene expression product from said first and/or second and/or third and/or fourth and/or fifth and/or sixth set of gene isoforms.

86.-91. (canceled)

92. The method of claim 84, wherein the gene expression products are derived from a tumor sample, e.g., a preparation of a primary tumor, metastatic tumor, lymph node, circulating tumor cells, ascites, or pleural effusion, plasma, serum, circulating, and interstitial fluid levels.

93.-94. (canceled)

95. The method of claim 84, wherein the value is compared to a reference standard, e.g., the level of expression of a control gene in the tumor sample.

96.-106. (canceled)

107. A reaction mixture comprising:
a plurality of detection reagents; and
a plurality of target nucleic acid molecules derived from a subject, wherein each of the plurality of detection reagents comprises a plurality probes to measure the level of gene expression product of a plurality of genes selected from a first and/or second and/or third and/or fourth and/or fifth and/or sixth set of gene isoforms, wherein:
(i) said first set of gene isoforms comprises or consists of genes in Table 8,
(ii) said second set of gene isoforms comprises or consists of gene isoforms in Table 9; and
(iii) said third set of gene isoforms comprises or consists of gene isoforms in Table 10; and
(iv) said fourth set of gene isoforms comprises or consists of gene isoforms in Table 11; and
(v) said fifth set of gene isoforms comprises or consists of gene isoforms in Table 12; and
(vi) said sixth set of gene isoforms comprises or consists of gene isoforms in Table 13.

108. The reaction mixture of claim 107, wherein each probe comprises a DNA, RNA or mixed DNA/RNA molecule, which is complementary to a nucleic acid sequence on each of the plurality of target nucleic acid molecules, wherein each target nucleic acid molecule is derived from a gene in said first and/or second and/or third and/or fourth and/or fifth and/or sixth set of gene isoforms.

109.-139. (canceled)
